(19) United States
(12) Patent Application Publication
(10) Pub. No.: US 2021/0278399 A1
(43) Pub. Date: Sep. 9, 2021

(54) SYSTEMS AND METHODS FOR DETECTION OF TARGET ANALYTES USING SELECTIVELY CLEAVABLE BONDS

(71) Applicant: Indiana Biosciences Research Institute, Inc, Indianapolis, IN (US)

(72) Inventors: Michael Joseph Pugia, Zionsville, IN (US); Zane Baird, Bloomington, IN (US)

(73) Assignee: Indiana Biosciences Research Institute, Inc, Indianapolis, IN (US)

(21) Appl. No.: 16/808,790

(22) Filed: Mar. 4, 2020

(51) Int. Cl. G01N 33/543; G01N 33/50; G01N 33/58; C07D 495/04; G01N 21/64; G01N 15/02

(52) U.S. Cl. CPC G01N 33/54313; G01N 33/5005; G01N 33/58; G01N 2021/6439; G01N 21/6428; G01N 15/0272; C07D 495/04

(57) ABSTRACT

The invention described herein is directed to methods of isolation and detection of target analytes in a sample. The target analytes are coupled to analyte detection particles which comprise base particles having labels and affinity agents coupled thereto by linker arms. The linker arms form bonds with the labels and target analytes and are cleavable under different label and affinity cleavable conditions. Systems and methods for preparing and using the analyte detection particles are also disclosed.

### FIELD

This disclosure relates to systems and method for the use of analyte detection particles for the detection of target analytes, including target molecules and target cells, in samples suspected of including the target analytes. The disclosure further relates to analyte detection particles and methods which include different affinity agents to attach labels and target analytes to base particles, which allows for isolation of the resulting complexes. The present disclosure provides a description with respect to the detection of analytes in the medical fields. It is to be understood, however, that the disclosed systems and methods are not limited in this respect as they have utility for a much broader scope of target analytes.

### BACKGROUND

The detection of target analytes is an important aspect of many scientific endeavors. A wide variety of analytes may be the subject of such detection methods and systems. In a particular aspect, for example, the detection of analytes in biological samples is important to the understanding and treatment of various medical conditions. Methods and systems have been described for the detection of such analytes.

Rare molecules are molecules which occur in the range of 1 to 50,000 copies per 10 μL or less of a liquid sample. The detection of rare molecules cannot be achieved by conventional affinity assays, which require molecular copy numbers far above those found for rare molecules. For example, immunoassays cannot typically achieve a detection limit of 1 picomolar (pM) or less. Immunoassays are limited by the affinity binding constant of an antibody, which is typically not higher than 10⁻¹² (1 pM). Immunoassays require at least a 100-fold antibody excess as the off-rate is generally 10⁻¹³ and a complete binding of all analyte in a sample is limited by antibody solubility. This same issue of antibody solubility prevents conventional immunoassays from reaching sub-attomolar detection levels.

The detection of rare molecules that are cell-bound or contained within a cell is also important in medical applications, such as in the diagnosis of diseases that can be propagated from a single cell. The detection of circulating rare molecules is complicated by the sample containing a mixture of rare and non-rare molecules. The materials can be cellular, e.g. internal to cells, or “cell free” material not bound to or associated with any intact cell. Cell free rare molecules are important in medical applications such as, for example, diagnosis of cancer in tissues. In the case of cancer, rare molecules are shed from tissues into circulation. It is understood that cell free rare molecules correlate to the total amount of rare molecules in diseased tissues, for example tumors, distributed throughout the body.

Analysis of cell free molecules requires isolation and detection of circulating rare molecules from a very small fraction of all molecules in a sample. When cell free molecules are shed into the peripheral blood from diseased cells in tissues, these molecules are mixed with molecules shed from healthy cells. For example, approximately 109 cells are present in 1 cm³ of diseased tissue. If this tissue mass was fully dissolved into 5 L of blood (blood volume of an average adult), this would only be 2 million cells per 10 mL of blood. This would be considered rare, considering that there are an average of 75 million leukocytes and 50 billion erythrocytes per 10 mL of blood, each of which releases non-rare molecules.

In another aspect, the detection of target analytes is complicated by the fact that the analyte may be represented in a sample in various forms. For example, the complexity of peptide and protein variations in samples causes significant issues when measurements of the respective peptides and proteins are desired. These issues of variation have been demonstrated using the SELDI affinity mass spectroscopic method in a study which utilized antibodies for peptide and protein isolation (Pugia, Glycoconj J, 2007). Peptides and proteins are known to fragment and to undergo post-translational modifications in biological systems under the action of enzymes. For example, a high degree of variations of urinary trypsin inhibitor has been detected in biological samples of different patients as the result of fragmentation and glyco-conjugation, with hundreds of different forms detected.

These variations cause problems for analysis. For example, the measurement of separate, unique fragments originating from the same peptide or protein often produces differing results. Determination of which fragments are more or less significant is needed, a summation of similar fragments might be required, and affinity reagents used for these methods can be more or less reactive to certain fragments. The variations of peptides and proteins increase as these variants become bound by other biomolecules which can alter the function of the variants.

The high degree of variations in peptides and proteins becomes a problem as immunoassay methods must often be able detect each variant independently. Sandwich immunoassays are typically used for specifically measuring unique fragments or forms of an analyte and rely on measuring a variation by binding two separate locations. Sandwich immunoassays require adequate space for two separate antibodies to bind the same fragment. However, as these fragments contain the same peptide or protein regions as the other variants, regions are often unsuited for binding to antibodies for specific assays.

Additional binding by other biomolecules can be blocking to antibodies or cause cross-reactivity. For example, cysteine may form disulfide bonds and other secondary molecules can bind fragments or be cleaved and alter antibody binding, to name a few of the problems in the measurement of peptides and proteins with a high degree of variation by immunoassay. Multiplexing is another problem for immunoassay methods as most methods use optical detection labels—whether chemiluminescent, fluorescent, or colorimetric—which provide a limited number of resolvable signals for simultaneous measurement within the same analysis. For this reason, analysis of hundreds to thousands of variations is a problem for optical systems. These methods require multiple, separate measurements in multiplexed panels and arrays, which increases cost and complexity.

Common alternative approaches to solve the problem of high degrees of variations use the peptide or protein to be measured as a substrate for the action of enzymes, proteases and peptidases. These measurements are based on the observed protease activity and can be used to measure the enzymes, proteases, peptidases and inhibitors thereof. For example, these methods have been used to analyze serine proteases of the trypsin family (Elastase, Cathepsin, Tryptase, Trypsin, Kallikrein, Thrombin, Plasmin and Factors VII & X) and their inhibitors (Bikunin, Uristatin, and Urinary Trypsin Inhibitor) (Corey U.S. Pat. No. 6,955,921). In these cases, the peptide is used as a substrate, attached to a chromophore at the amino acid cleavage site. Upon cleavage by the protease, a fragment is released and activated to generate a color. The concentration of inhibitor is measured when a known amount of protease is added. Here the amount of inhibitor is inversely proportional to the amount of substrate released, since the inhibitor decreases the activity of protease. The chromophores however are sensitive to interference where color is reversed or prematurely generated by sample pH, oxidants, reductants, or reactants.

Mass spectroscopy to measure the peptide or protein substrates has been used to eliminate the issues associated with chromophores. For example, this has been shown for the renin-angiotensin-aldosterone system. In this system angiotensinogen I (Ang I) (DRVYIHPFHL) is converted to Ang II (DRVYIHPF) by the cleavage of two C-terminal amino acids in an enzymatic cleavage by renin (Popp 2014). Measurement of Ang I allows for a plasma renin activity assay by utilizing anti-Ang I antibodies immobilized to affinity particles to simultaneously capture endogenous Ang I from plasma along with a stable isotope-labeled Ang I. The plasma sample is split and incubated either at 37° C. for 3 h, or on ice. A determination of the difference in Ang I concentration for the two plasma incubation conditions allows the calculation of the patient's plasma renin activity. This enzyme protease and peptidase assay is still sensitive to interference where activities are inhibited or activated by sample pH, sample stability, inhibitors, co-factors, time and temperature.

Mass spectrometry (MS) is an extremely sensitive and specific technique very well suited for detecting small molecules down to pM concentrations with small sample consumption (1 μL or less). Mass spectroscopy also has the ability to simultaneously measure hundreds of components (multiplexing) present in complex biological media in a single assay without the need for labeled reagents. The method offers specificity and sensitivity until the biological complexity causes overlapping signals (isobaric interference) or results in ion suppression. The coupling of mass spectroscopy with a pre-separation step such as liquid chromatography (LC-mass spectroscopy) is a widely used method of increasing sensitivity and limiting isobaric interference, and overcoming ion suppression by high abundance non-analyte sample components. However, this greatly increases analytical run time, cost, and sample preparation complexity.

Tandem mass spectroscopy (MS/MS) can be used to increase signal-to-noise in the case of high background interference, as well as to distinguish isobaric analytes sharing the same parent mass-to-charge (m/z), but exhibiting unique fragmentation within the mass spectrometer. However, analysis of MS/MS data is not a simple task, especially in the case of post-translationally modified peptides and proteins, and still suffers the effects of ion suppression, especially in the case of poorly ionizable fragments. Matrix-assisted laser desorption/ionization using a time-of-flight mass spectrometer (MALDI-TOF) is well suited for high sensitivity analysis of low abundance molecules; however, sample complexity and matrix interference frequently result in isobaric interference.

The current state of mass spectroscopy is not competitive with routine clinical diagnostic systems, with noted problems in the inability to separate markers of interest (sample preparation), loss of sensitivity due to high background in clinical samples, inefficient ionization of some fragments, and isobaric interference in complex samples such as blood. In addition, mass spectroscopy is often unable to detect certain masses due to ion suppression by more easily ionizable molecules present in the sample. These issues typically cause false results. A proteolytic digestion is often utilized for the analysis and quantitation of proteins and peptides by mass spectroscopy. The digestion serves to break the protein or peptide into smaller, more easily detectable fragments that can be better separated before mass spectroscopy analysis, as is the case with LC-mass spectroscopy. While serving to increase analytical sensitivity, proteolytic digestion is often not reproducible—not all proteins and bound forms can be fragmented, certain fragments are not easily detected (the method is biased towards easily ionizable fragments), various matrix components can inhibit the digestion enzymes used, and redundant amino acid sequences can result in ambiguity during data analysis. Fragments detected under these conditions often do not relate to the clinical state as they are not the relevant molecule regions. Additionally, quantitation of fragments requires the inclusion of a stable isotope internal standard.

One approach to solve the problems of sensitivity and quantitation by mass spectroscopy is to chemically add a label to the molecule to be measured. This mass labeling approach has been helpful in the detection of cells, tissues, peptides, and proteins by mass spectrometry. Chemical labeling works by introducing a charged group of known mass directly on the molecule to be measured through a chemical reaction. While these mass labeling approaches allow masses to be more easily ionized and uniquely identified, they still suffer from the effects of isobaric interference, require the analyte to have a functional group amenable to mass label introduction, and are limited by the mass of the analyte to be measured. Therefore, other approaches have been sought to avoid or reduce the problems associated with these mass spectral analysis methods.

One approach utilizes affinity agents to capture an analyte and remove contaminants prior to detection by mass spectroscopy, often termed affinity mass spectrometry. One method of affinity mass spectrometry is Surface Enhanced Laser Desorption and Ionization or SELDI (U.S. Pat. Nos. 5,719,060 and 6,225,047). This method uses affinity agents to specifically absorb analytes to a surface which aids in the ionization of captured molecules (Zhu 2006).

Other examples include affinity agents on a solid substrate, either flexible or rigid, that has a sample-presenting surface. Other affinity mass spectrometry methods use an affinity agent, such as an antibody, attached to a capture surface or particle for isolation, followed by ionization. While these methods have been successfully used for clinical measurement, they often require enzymatic digestion in order to produce fragments detectable by mass spectroscopy. This method of sample preparation remains a difficult and complex multistep process to automate and is noncompetitive with other detection technologies used in the clinical laboratory.

A mass labeling approach which utilizes affinity agents has been accomplished through the coupling of metals to antibodies against rare cell molecules of interest (Bandura 2009, Lee 2008). In this instance the entire sample was subjected to atomization and the metal content was used to assay the presence of the rare molecules, which resulted in the destruction of the entire sample. In Pugia PCT/US2015/033278, a quaternary ammonium compound was attached to a nanoparticle through disulfide bonds. The nanoparticle was also conjugated to affinity agents for rare molecules. A chemical was used as an “alteration agent” to release the mass label from the affinity agent by breaking a disulfide bond, namely dithiothreitol (DTT) or tris(2-carboxyethyl)phosphine (TCEP). This method allowed sensitivities in the μM range to detect a limited number of peptide and protein variants in a sample.

Combining affinity agents and mass labeling for mass spectrometry using a nanoparticle and mass label is shown in Cooks PCT/US16/53610 filed Sep. 24, 2016. In this example, an affinity tag and a mass label with a quaternary ammonium group was connected to a particle by a cleavable ketal linkage. This method used the affinity tag to connect to an affinity agent. While this method allowed high sensitivities in nM range to detect limited numbers of peptide and protein variants in a sample, it suffers from a lack of specificity due to the affinity tag binding to non-analyte molecules. This made the method unable to accurately measure all the variations of an analyte and therefore resulted in false positives.

Some labeling strategies such as isobaric tags for relative and absolute quantitation (iTRAQ™, SCIEX), or tandem mass tags (TMT™, Thermo Scientific), offer a direct labeling approach that is amenable to multiplexed sample measurement and relative quantitation. In both iTRAQ and TMT, separate proteolytic digests are reacted with reagents which introduce unique charged groups onto N-terminal amino acids, as well as cysteine, lysine, and carbonyl moieties. The labeled samples are then pooled and analyzed in the same LC-mass spectroscopy run. The result is a multiplexable assay capable of relative quantification within the same LC-mass spectroscopy analysis. The reagents enable multiplexing by producing isobaric, chromatographically indistinguishable, derivatized peptides which produce unique reporter ions for identical peptides from different samples analyzed in the same pool. As this method still relies on pre-separation by LC, proteolytic digestion, as well as the added complexity of independent sample derivatization, it is subject to the same problems associated with the previously discussed methods.

The field requires an improved method capable of detecting all variations of peptides and proteins in a sample. This method should not be dependent on further enzymatic processing or peptidase reactions, and should be able to measure any and all variations of an analyte in a single determination. A new method which combines affinity agents and analytical labeling must be sensitive to variations of peptides and proteins in a sample and allow for consistent measurement across patients and samples.

### SUMMARY

In one embodiment, there is provided an analyte detection particle for detection of target analytes. The analyte detection particle includes a base particle, a label and an affinity agent for a target analyte. The label is attached to the base particle by a label linker arm which is coupled to the base particle, and is joined to the label by a label bond that is selectively cleavable to separate the label from the particle. The affinity agent is attached to the base particle by an affinity linker arm which is coupled to the particle, and is joined to the affinity agent by an affinity bond that is selectively cleavable to separate the target analyte from the base particle. At least one of the label bond and the affinity bond is cleavable under conditions which do not cleave the other bond, and which leave the label and/or target analyte viable for analysis.

The detection methods disclosed herein include incubating a sample suspected of containing the target analyte with the analyte detection particles. Target analytes couple with the affinity agent, forming a complex which may be collected separate from the other components of the sample. In one aspect, the label is detected while still coupled with the analyte detection particle. In another aspect, the label is cleaved from the analyte detection particle before being detected. The target analyte may be cleaved from the analyte detection particle either before or after detection and/or cleavage of the label.

In another embodiment, there is provided an analyte collection particle for collection of target analytes. The analyte collection particle includes a base particle, a collection particle, and an affinity agent for the target analyte. The capture particle has a property which facilitates collection of the ACP, such as by being readily separated by centrifugation, having a relatively large retention size or being magnetic. The collection particle is attached to the base particle by an affinity linker arm which is coupled with the particle, and is joined to the collection particle by a collection bond that may be cleavable to separate the collection particle from the base particle. The affinity agent is attached to the base particle by an affinity linker arm which is coupled to the particle, and is joined to the affinity agent by an affinity bond that is selectively cleavable to separate the target analyte from the base particle. At least one of the collection bond and the affinity bond is cleavable under conditions which do not cleave the other bond, and which leave the target analyte viable for analysis.

The collection methods disclosed herein include incubating a sample suspected of containing the target analyte(s) with the analyte collection particles. Target analytes couple with the affinity agent(s), forming a complex(es) which may be separated from the other components of the sample. In one aspect, the target analyte is detected while still coupled with the analyte collection particle. In another aspect, the target analyte is cleaved from the analyte collection particle before being detected. The collection particle may be cleaved from the analyte collection particle either before or after detection and/or cleavage of the target analyte.

In another embodiment, a detection method comprises incubating the sample suspected of containing the target analyte(s) with both analyte detection particles and analyte collection particles. This results in complexes wherein target analytes are coupled with both analyte detection particles and analyte collection particles. The presence of the collection particle facilitates the collection of the target analytes. The detection of the target analytes proceeds in accordance with detection methods as when the analyte collection particles are not used.

In another aspect, the label bonds, collection bonds and affinity bonds may be cleaved under differing conditions. For example, the label bonds are cleaved under label cleavage conditions which differ from those which cleave the collection bonds and/or affinity bonds. Similarly, the collection bonds are cleaved under collection cleavage conditions which differ from those which cleave the label bonds and/or affinity bonds, and the affinity bonds are cleaved under affinity cleavage conditions which differ from those which cleave the label bonds and/or collection bonds.

Further embodiments are described herein. For example, disclosed methods have particular utility for enriching and detecting rare target molecules and rare target cells. Also, provisions are made for amplifying the signal that is detected, which further enhances the ability to detect analytes that are present in relatively low amounts. This is accomplished, for example, by including multiple labels in a single analyte detection particle. In other aspects, the embodiments provide for collection and detection of more than one different target analyte at the same time. The different target analytes may be unrelated, or they may be variations of a target analyte.

Further forms, objects, features, aspects, benefits, advantages, and embodiments of the present invention will become apparent from the detailed description and drawings provided herewith.

### BRIEF DESCRIPTION OF THE DRAWINGS

FIG. 1 is schematic drawing showing an example of the formation of target variants by fragmentation, addition, or binding.

FIG. 2 is a schematic drawing showing a nanoparticle including three amide functional groups attached thereto.

FIG. 3 is a schematic drawing of an analyte detection particle including linker arms linking both a label and an affinity agent to the base nanoparticle.

FIG. 4 is a schematic drawing showing the binding of a specific target analyte to a labeled nanoparticle and the subsequent cleavage of the labels.

FIG. 5 is a schematic drawing showing the binding of multiple target analytes to a labeled nanoparticle and the subsequent cleavage of the labels.

FIG. 6 is a schematic drawing showing the binding of multiple target analytes to multiple labeled nanoparticles and the subsequent cleavage of the labels.

FIG. 7 is a schematic drawing showing a nanoparticle having an attached affinity agent and an attached label. As described in the text, the label may comprise any of an electrochemical, optical or mass label.

FIG. 8 is a schematic drawing showing a cell assay in which particles without bound antibodies pass through a membrane, while particles with bound antibodies are retained on the membrane.

FIG. 9 is a schematic drawing showing the cleavage of a label from an analyte detection particle for measurement.

FIG. 10 is a schematic drawing showing the acid cleavage of a collection complex.

FIG. 11 is a schematic drawing showing the retention of SKBR cells when bound to a capture bead.

FIG. 12 is a schematic drawing showing the cleavage of the collection bond for separation of the SKBR cells from the capture particle.

FIG. 13 is an image showing collection particles containing captured SKBR cells.

FIG. 14 is an image showing collection particles not containing captured SKBR cells.

FIG. 15 is a schematic drawing showing both analyte detection particles and collection particles coupling with a target analyte, e.g. SKBR cells, and also the size exclusion separation of the target analyte.

FIG. 16 is a schematic drawing showing the use of analyte detection particles including an electrochemical label and a mass label.

FIG. 17 is a schematic drawing showing the use of an electrochemical detectable label to identify the presence of target cells, indicating a follow up step of releasing the coupled target cells for additional analysis.

The drawings herein are provided to facilitate the understanding of the principles described herein, and are provided by way of illustration and not limitation on the scope of the appended claims. The drawings are not to scale.

### DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Certain embodiments of the invention are shown in detail, but some features that are well known, or that are not relevant to the present invention, may not be shown for the sake of conciseness and clarity.

### I. Target Analytes

The materials and methods described herein are useful with any of a broad variety of target analytes which may be suitably coupled to particles as disclosed herein. The target analytes include a wide range of target molecules and target cells. In addition, the target analytes may comprise one or more target variants, as described hereafter.

Rare Molecules are molecules of interest that occur in a sample at a very low concentration. For example, a sample may include rare molecules in the range of 1 to 50,000 copies per μL (femtomolar (fM)) or less. Rare cells are cells that are present in a sample in relatively small quantities compared to the amount of non-rare cells in the sample. For example, rare cells may be present in a sample in an amount of about 10⁻⁸% to about 10⁻²% by weight of the total cell population in the sample. These rare molecules and rare cells are collectively referred to as target rare analytes. There are particular advantages of the materials and methods disclosed herein in the ability and accuracy of detecting target rare analytes.

### A. Target Molecules

The term “target molecules” refers generally to molecules of interest that may be detected as analytes in a sample. The target molecules may be contained within or bound to cells, or they may be “cell free molecules” which freely circulate in the sample. Following is an exemplary list of target molecules for which the present materials and methods are useful.

A given test may have a specific target molecule as being of interest. Alternatively, a test may seek to identify at the same time a population of molecules. The population of molecules may include related or unrelated molecules. Related molecules may comprise a group of molecules that share a common portion of molecular structure that specifically defines the group of molecules as being molecules of interest. The common portion distinguishes the group of molecules from other molecules. The related molecules may be target variants, which term refers to a part, piece, fragment or other derivation or modification of a target molecule.

Cell free molecules include biomolecules useful in medical diagnosis and treatment of diseases. Medical diagnosis of diseases includes, but is not limited to, the use of biomarkers for detection of cancer, cardiac damage, cardiovascular disease, neurological disease, hemostasis/hemastasis, fetal maternal assessment, fertility, bone status, hormone levels, vitamins, allergies, autoimmune diseases, hypertension, kidney disease, metabolic disease, diabetes, liver diseases, infectious diseases and other biomolecules useful in medical diagnosis of diseases, for example.

The samples to be analyzed are ones that are suspected of containing the target molecules. The samples may be biological samples or non-biological samples. Biological samples may be from a plant, animal, protist or other living organism, including Animalia, fungi, plantae, chromista, or protozoa or other eukaryote species or bacteria, archaea, or other prokaryote species. Non-biological samples include aqueous solutions, environmental, products, chemical reaction production, waste streams, foods, feed stocks, fertilizers, fuels, and the like.

Biological samples include biological fluids such as whole blood, serum, plasma, sputum, lymphatic fluid, semen, cells, exosomes, endosomes, extracellular vesicles, lipids, extracellular matrix, interstitial fluid, mucus, vaginal secretions, nasal secretions, feces, urine, spinal fluid, saliva, stool, cerebral spinal fluid, tears, or tissues for example. Biological tissues include, by way of illustration, hair, skin, or sections or excised tissues from organs or other body parts. For example, the target molecules may be from various tissue sources, including: the lung, bronchus, colon, rectum, extra cellular matrix, dermal, vascular, stem, lead, root, seed, flower, pancreas, prostate, breast, liver, bile duct, bladder, ovary, brain, central nervous system, kidney, pelvis, uterine corpus, oral cavity or pharynx or cancers. In many instances, the sample is aqueous, such as urine, whole blood, plasma or serum samples, while in other instances the sample must be made into a solution or suspension for testing.

Target molecules of metabolic interest further include, but are not limited to, those that impact the concentration of ACC Acetyl Coenzyme A Carboxylase, Adpn Adiponectin, AdipoR Adiponectin Receptor, AG Anhydroglucitol, AGE Advance glycation end products, Akt Protein kinase B, AMBK pre-alpha-1-microglobulin/bikunin, AMPK 5′-AMP activated protein kinase, ASP Acylation stimulating protein, Bik Bikunin, BNP B-type natriuretic peptide, CCL Chemo-kine (C-C motif) ligand, CINC Cytokine-induced neutrophil chemoattractant, CTF C-Terminal Fragment of Adiponectin Receptor, CRP C-reactive protein, DGAT Acyl CoA diacylglycerol transferase, DPP-IV Dipeptidyl peptidase-IV, EGF Epidermal growth factor, eNOS Endothelial NOS, EPO Erythropoietin, ET Endothelin, Erk Extracellular signal-regulated kinase, FABP Fatty acid-binding protein, FGF Fibroblast growth factor, FFA Free fatty acids, FXR Farnesoid X receptor a, GDF Growth differentiation factor, GH Growth hormone, GIP Glucose-dependent insulinotropic polypeptide, GLP Glucagon-like peptide-1, GSH Glutathione, GHSR Growth hormone secretagogue receptor, GULT Glucose transporters, GCD59 glycated CD59 (aka glyCD59), HbA1c Hemogloblin A1c, HDL High-density lipoprotein, HGF Hepatocyte growth factor, HIF Hypoxia-inducible factor, HMG 3-Hydroxy-3-methylglutaryl CoA reductase, I-α-I Inter-α-inhibitor, Ig-CTF Immunoglobulin attached C-Terminal Fragment of AdipoR, insulin, IDE Insulin-degrading enzyme, IGF Insulin-like growth factor, IGFBP IGF binding proteins, IL Interleukin cytokines, ICAM Intercellular adhesion molecule, JAK STAT Janus kinase/signal transducer and activator of transcription, JNK c-Jun N-terminal kinases, KIM Kidney injury molecule, LCN-2 Lipocalin, LDL Low-density lipoprotein, L-FABP Liver type fatty acid binding protein, LPS Lipopolysaccharide, Lp-PLA2 Lipoprotein-associated phospholipase A2, LXR Liver X receptors, LYVE Endothelial hyaluronan receptor, MAPK Mitogen-activated protein kinase, MCP Monocyte chemotactic protein, MDA Malondialdehyde, MIC Macrophage inhibitory cytokine, MIP Macrophage infammatory protein, MMP Matrix metalloproteinase, MPO Myeloperoxidase, mTOR Mammalian of rapamycin, NADH Nicotinamide adenine di-nucleotide, NGF Nerve growth factor, NFκB Nuclear factor kappa-light-chain-enhancer of activated B cells, NGAL Neutrophil gelatinase lipocalin, NOS Nitric oxide synthase NOX NADH oxidase NPY Neuropeptide Yglucose, insulin, proinsulin, c peptide OHdG Hydroxy-deoxyguanosine, oxLDL Oxidized low density lipoprotein, P-α-I pre-interleukin-α-inhibitor, PAI-1 Plasminogen activator inhibitor, PAR Protease-activated receptors, PDF Placental growth factor, PDGF Platelet-derived growth factor, PKA Protein kinase A, PKC Protein kinase C, PI3K Phosphatidylinositol 3-kinase, PLA2 Phosphatidylinositol 3-kinase, PLC Phospholipase C, PPAR Peroxisome proliferator-activated receptor, PPG Postprandial glucose, PS Phosphatidyl-serine, PR Protienase, PYY Neuropeptide like peptide Y, RAGE Receptors for AGE, ROS Reactive oxygen species, S100 Calgranulin, sCr Serum creatinine, SGLT2 Sodium-glucose transporter 2, SFRP4 secreted frizzled-related protein 4 precursor, SREBP Sterol regulatory element binding proteins, SMAD Sterile alpha motif domain-containing protein, SOD Superoxide dismutase, sTNFR Soluble TNF α receptor, TACE TNFα alpha cleavage protease, TFPI Tissue factor pathway inhibitor, TG Triglycerides, TGF β Transforming growth factor-β, TIMP Tissue inhibitor of metalloproteinases, TNF α Tumor necrosis factors-α, TNFR TNF α receptor, THP Tamm-Horsfall protein, TLR Toll-like receptors, TnI Troponin I, tPA Tissue plasminogen activator, TSP Thrombospondin, Uri Uristatin, uTi Urinary trypsin inhibitor, uPA Urokinase-type plasminogen activator, uPAR uPA receptor, VCAM Vascular cell adhesion molecule, VEGF Vascular endothelial growth factor, and YKL-40 Chitinase-3-like protein.

Target molecules of interest that are highly expressed by pancreatic tissue or found in the pancreas include insulin, proinsulin, c-peptide, PNLIPRP1 pancreatic lipase-related protein 1, SYCN syncollin, PRSS1 protease, serine, 1 (trypsin 1) Intracellular, CTRB2 chymotrypsinogen B2 Intracellular, CELA2A chymotrypsin-like elastase family, member 2A, CTRB1 chymo-trypsinogen B1 Intracellular, CELA3A chymotrypsin-like elastase family, member 3A Intra-cellular, CELA3B chymotrypsin-like elastase family, member 3B Intracellular, CTRC chymo-trypsin C (caldecrin), CPA1 carboxypeptidase A1 (pancreatic) Intracellular, PNLIP pancreatic lipase, and CPB1 carboxypeptidase B1 (tissue), AMY2A amylase, alpha 2A (pancreatic), PDX1 insulin promoter factor 1, MAFA Maf family of transcription factors, GLUT2 Glucose Transporter Type 2, ST8SIA1 Alpha-N-acetylneuraminide alpha-2,8-sialyltransferase, CD9 tetraspanin, ALDH1A3 aldehyde dehydrogenase, CTFR cystic fibrosis transmembrane conductance regulator as well as diabetic auto immune antibodies such as against GAD, IA-2, IAA, ZnT8 or the like.

Some specific examples of therapeutic proteins and peptides include glucagon, ghrelin, leptin, growth hormone, prolactin, human placental, lactogen, luteinizing hormone, follicle stimulating hormone, chorionic gonadotropin, thyroid stimulating hormone, adrenocorticotropic hormone, vasopressin, oxytocin, angiotensin, parathyroid hormone, gastrin, buserelin, antihemophilic factor, pancrelipase, insulin, insulin aspart, porcine insulin, insulin lispro, insulin isophane, insulin glulisine, insulin detemir, insulin glargine, immunglobulins, interferon, leuprolide, denileukin, asparaginase, thyrotropin, alpha-1-proteinase inhibitor, exenatide, albumin, coagulation factors, alglucosidase alfa, salmon calcitonin, vasopressin, dpidermal growth factor (EGF), cholecystokinin (CCK-8), vacines, human growth hormone and others. Some new examples of therapeutic proteins and peptides include GLP-1-GCG, GLP-1-GIP, GLP-1, GLP-1-GLP-2, and GLP-1-CCKB′.

Target molecules of interest that are highly expressed by adipose tissue include, but are not limited to, ADIPOQ Adiponectin, C1Q and collagen domain containing, TUSC5 Tumor suppressor candidate 5, LEP Leptin, CIDEA Cell death-inducing DFFA-like effector a, CIDEC Cell death-inducing DFFA-like effector C, FABP4 Fatty acid binding protein 4, adipocyte, LIPE, GYG2, PLIN1 Perilipin 1, PLIN4 Perilipin 4, CSN1S1, PNPLA2, RP11-407P15.2 Protein LOC100509620, L GALS12 Lectin, galactoside-binding, soluble 12, GPAM Glycerol-3-phosphate acyltransferase, mitochondrial, PR325317.1 predicted protein, ACACB Acetyl-CoA carboxylase beta, ACVR1C Activin A receptor, type IC, AQP7 Aquaporin 7, CFD Complement factor D (adipsin)m CSN1S1Casein alpha s1, FASN Fatty acid synthase GYG2 Glycogenin 2 KIF25Kinesin family member 25 LIPELipase, hormone-sensitive PNPLA2 Patatin-like phospholipase domain containing 2 SLC29A4 Solute label family 29 (equilibrative nucleoside transporter), member 4 SLC7A10 Solute label family 7 (neutral amino acid transporter light chain, asc system), member 10, SPX Spexin hormone and TIMP4 TIMP metallopeptidase inhibitor 4.

Target molecules of interest that are highly expressed by the adrenal gland and thyroid include, but are not limited to, CYP11B2 Cytochrome P450, family 11, subfamily B, polypeptide 2, CYP11B1 Cytochrome P450, family 11, subfamily B, polypeptide 1, CYP17A1 Cytochrome P450, family 17, subfamily A, polypeptide 1, MC2R Melanocortin 2 receptor (adreno-corticotropic hormone), CYP21A2 Cytochrome P450, family 21, subfamily A, polypeptide 2, HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine betamono-oxygenase), HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cyto-chrome P450, family 11, subfamily A, polypeptide 1, DBH Dopamine beta-hydroxylase (dop-amine beta-monooxygenase), AKR1B1 Aldo-keto reductase family 1, member B1 (aldose reductase), NOV Nephroblastoma overexpressed, FDX1 Ferredoxin 1, DGKK Diacylglycerol kinase, kappa, MGARP Mitochondria-localized glutamic acid-rich protein, VWA5B2 Von Willebrand factor A domain containing 5B2, C18orf42 Chromosome 18 open reading frame 42, KIAA1024, MAP3K15 Mitogen-activated protein kinase kinase kinase 15, STAR Steroidogenic acute regulatory protein Potassium channel, subfamily K, member 2, NOV nephroblastoma overexpressed, PNMT phenylethanolamine N-methyltransferase, CHGB chromogranin B (secretogranin 1), and PHOX2A paired-like homeobox 2a.

Target molecules of interest that are highly expressed by bone marrow include, but are not limited, to DEFA4 defensin alpha 4 corticostatin, PRTN3 proteinase 3, AZU1 azurocidin 1, DEFA1 defensin alpha 1, ELANE elastase, neutrophil expressed, DEFA1B defensin alpha 1B, DEFA3 defensin alpha 3 neutrophil-specific, mass spectroscopy 4A3 membrane-spanning 4-domains, subfamily A, member 3 (hematopoietic cell-specific), RNASE3 ribonuclease RNase A family 3, MPO myeloperoxidase, HBD hemoglobin, delta, and PRSS57 protease, serine 57.

Target molecules of interest that are highly expressed by the brain include, but are not limited to, GFAP glial fibrillary acidic protein, OPALIN oligodendrocytic myelin paranodal and inner loop protein, OLIG2 oligodendrocyte lineage transcription factor 2, GRIN1glutamate receptor ionotropic, N-methyl D-aspartate 1, OMG oligodendrocyte myelin glycoprotein, SLC17A7 solute label family 17 (vesicular glutamate transporter), member 7, C1orf61 chromosome 1 open reading frame 61, CREG2 cellular repressor of E1A-stimulated genes 2, NEUROD6 neuronal differentiation 6, ZDHHC22 zinc finger DHHC-type containing 22, VSTM2B V-set and transmembrane domain containing 2B, and PMP2 peripheral myelin protein 2.

Target molecules of interest that are highly expressed by the endometrium, ovary, or placenta include, but are not limited to, MMP26 matrix metallopeptidase 26, MMP10 matrix metallopeptidase 10 (stromelysin 2), RP4-559A3.7 uncharacterized protein and TRH thyrotropin-releasing hormone. Rare molecules of interest that are highly expressed by the gastrointestinal tract, salivary gland, esophagus, stomach, duodenum, small intestine, or colon include, but are not limited to, GKN1 Gastrokine 1, GIF Gastric intrinsic factor (vitamin B synthesis), PGA5 Pepsinogen 5 group I (pepsinogen A), PGA3 Pepsinogen 3, group I (pepsinogen A, PGA4 Pepsinogen 4 group I (pepsinogen A), LCT Lactase, DEFA5 Defensin, alpha 5 Paneth cell-specific, CCL25 Chemokine (C-C motif) ligand 25, DEFA6 Defensin alpha 6 Paneth cell-specific, GAST Gastrin, mass spectroscopy 4A10 Membrane-spanning 4-domains subfamily A member 10, ATP4A and ATPase, H+/K+ exchanging alpha polypeptide.

Target molecules of interest that are highly expressed by the heart or skeletal muscles include, but are not limited to, NPPB natriuretic peptide B, TNNI3 troponin I type 3 (cardiac), NPPA natriuretic peptide A, MYL7 myosin light chain 7 regulatory, MYBPC3 myosin binding protein C (cardiac), TNNT2 troponin T type 2 (cardiac) LRRC10 leucine rich repeat containing 10, ANKRD1 ankyrin repeat domain 1 (cardiac muscle), RD3L retinal degeneration 3-like, BMP10 bone morphogenetic protein 10, CHRNE cholinergic receptor nicotinic epsilon (muscle), and SBK2 SH3 domain binding kinase family member 2.

Target molecules of interest that are highly expressed by the kidney include, but are not limited to, UMOD uromodulin, TMEM174 transmembrane protein 174, SLC22A8 solute label family 22 (organic anion transporter) member 8, SLC12A1 solute label family 12 (sodium/-potassium/chloride transporter) member 1, SLC34A1 solute label family 34 (type II sodium/-phosphate transporter) member 1, SLC22A12 solute label family 22 (organic anion/urate transporter) member 12, SLC22A2 solute label family 22 (organic cation transporter) member 2, MCCD1 mitochondrial coiled-coil domain 1, AQP2 aquaporin 2 (collecting duct), SLC7A13 solute label family 7 (anionic amino acid transporter) member 13, KCNJ1 potassium inwardly-rectifying channel, subfamily J member 1 and SLC22A6 solute label family 22 (organic anion transporter) member 6.

Target molecules of interest that are highly expressed by the lung include, but are not limited to, SFTPC surfactant protein C, SFTPA1 surfactant protein A1, SFTPB surfactant protein B, SFTPA2 surfactant protein A2, AGER advanced glycosylation end product-specific receptor, SCGB3A2 secretoglobin family 3A member 2, SFTPD surfactant protein D, ROS1 proto-oncogene 1 receptor tyrosine kinase, mass spectroscopy 4A15 membrane-spanning 4-domains subfamily A member 15, RTKN2 rhotekin 2, NAPSA napsin A aspartic peptidase, and LRRN4 leucine rich repeat neuronal 4.

Target molecules of interest that are highly expressed by liver or gallbladder include, but are not limited to, APOA2 apolipoprotein A-II, A1BG alpha-1-B glycoprotein, AHSG alpha-2-HS-glycoprotein, F2 coagulation factor II (thrombin), CFHR2 complement factor H-related 2, HPX hemopexin, F9 coagulation factor IX, CFHR2 complement factor H-related 2, SPP2 secreted phosphoprotein 2 (24 kDa), C9 complement component 9, MBL2 mannose-binding lectin (protein C) 2 soluble and CYP2A6 cytochrome P450 family 2 subfamily A polypeptide 6. Rare molecules of interest that are highly expressed by testis or prostate include, but are not limited to, PRM2 protamine 2 PRM1 protamine 1 TNP1 transition protein 1 (during histone to protamine replacement), TUBA3C tubulin, alpha 3c LELP1late cornified envelope-like proline-rich 1 BOD1L2 biorientation of chromosomes in cell division 1-like 2 ANKRD7 ankyrin repeat domain 7 PGK2 phosphoglycerate kinase 2 AKAP4 A kinase (PRKA) anchor protein 4 TPD52L3 tumor protein D52-like 3 UBQLN3 ubiquilin 3 and ACTL7A actin-like 7A.

### B. Target Variants

In addition to testing for a particular target molecule, a test may also detect target variants which can instead, and/or in addition, be detected as a means for detecting the target molecule(s). The relevant variations of a target molecule constitute target variants. These target variants may be present naturally in the sample, or they may be intentionally produced. One or more target variants may be indicative of a particular population of target molecules. Target variants may be generated from parts and pieces of cells and tissues, as well as from small molecules. Binding and association reactions also lead to additional differences in target variants by generating bound forms which are variations that differ from unbound forms.

Target variants may comprise molecules of biological or non-biological origin, including small molecules such as metabolites, co-factors, substrates, amino acids, metals, vitamins, fatty acids, biomolecules, peptides, carbohydrates or others. Target variants may also include macromolecules, such as glycoconjugates, lipids, nucleic acids, polypeptides, receptors, enzymes and proteins, as well as cells and tissues including cellular structures, peroxisomes, endoplasmic reticulum, endosomes, exosomes, lysosomes, mitochondria, cytoskeleton, membranes, nucleus, extra cellular matrix or other molecules typically measured.

Target variants can be used to measure enzymes, proteases, peptidase, proteins and inhibitors acting to form the target variants. The target variants may be formed naturally, or may be man-made, such as biologicals, therapeutics or others. These target variants can result intentionally from fragmentation, additions, binding or other modifications of the analyte. Some examples in accordance with the principles described herein are directed to the addition of peptidases, enzymes, inhibitors or other reagents prior to the method of isolation such that variations of analyte are formed. These target variants can be the result of intentional affinity reactions to isolate target variants prior to analysis with the method.

In accordance with the principles described, target variants can be derived from a molecule of biological or non-biological origin. The target variants include but are not limited to biomolecules such as carbohydrates, lipids, nucleic acids, peptides and proteins. Target variants can be the result of reactions, biological processes, disease, or intentional reactions and can be used to measure diseases or natural states. Target variants can also result from changes in molecules, such as proteins, enzymes, biologics or peptides, of man-made or natural origin, and include bioactive and non-bioactive molecules such as those used in medical devices, therapeutic use, diagnostic use, used for measurement of processes, and those used as food, in agriculture, in production, as pro- or pre-biotics, in micro-organisms or cellular production, as chemicals for processes, for growth, measurement or control of cells, used for food safety and environmental assessment, used in veterinary products, and used in cosmetics. Target variants can be fragments of larger portions or bound forms and can be used to measure other molecules, such as enzymes, peptidase and others. The measurements of other molecules, such as enzymes, peptidase and others can be based on formation of target variants, such as enzymatic or proteolytic products. The measurements of other molecules, such as natural inhibitors, synthetic inhibitors and others, can be based on the lack of formation of target variants.

### C. Examples of Target Variants

Target molecule fragments that can be used to measure peptidases of interest include those in the MEROPS, which is an on-line database for peptidases (also known as proteases) and identifies ˜902,212 different sequences of aspartic, cysteine, glutamic, metallo, asparagine, serine, threonine and general peptidases catalytics types which are further categorized and include those listed for the following pathways: 2-Oxocarboxylic acid metabolism, ABC transporters, African trypanosomiasis, alanine, aspartate and glutamate metabolism, allograft rejection, Alzheimer's disease, amino sugar and nucleotide sugar metabolism, amoebiasis, AMPK signaling pathway, amyotrophic lateral sclerosis (ALS), antigen processing and presentation, apoptosis, arachidonic acid metabolism, arginine and proline metabolism, arrhythmogenic right ventricular cardiomyopathy (ARVC), asthma, autoimmune thyroid disease, B cell receptor signaling pathway, bacterial secretion system, basal transcription factors, beta-alanine metabolism, bile secretion, biosynthesis of amino acids, biosynthesis of secondary metabolites, biosynthesis of unsaturated fatty acids, biotin metabolism, bisphenol degradation, bladder cancer, cAMP signaling pathway, carbon metabolism, cardiac muscle contraction, cell adhesion molecules (CAMs), cell cycle, cell cycle—yeast, chagas disease (American trypanosomiasis), chemical carcinogenesis, cholinergic synapse, colorectal cancer, complement and coagulation cascades, cyanoamino acid metabolism, cysteine and methionine metabolism, cytokine-cytokine receptor interaction, cytosolic DNA-sensing pathway, degradation of aromatic compounds, dilated cardiomyopathy, dioxin degradation, DNA replication, dorso-ventral axis formation, drug metabolism—other enzymes, endocrine and other factor-regulated calcium reabsorption, endocytosis, epithelial cell signaling in Helicobacter pylori infection, Epstein-Barr virus infection, estrogen signaling pathway, Fanconi anemia pathway, fatty acid elongation, focal adhesion, folate biosynthesis, foxO signaling pathway, glutathione metabolism, glycerolipid metabolism, glycerophospholipid metabolism, glycosylphosphatidylinositol (GPI)-anchor bio-synthesis, glyoxylate and dicarboxylate metabolism, GnRH signaling pathway, graft-versus-host disease, hedgehog signaling pathway, hematopoietic cell lineage, hepatitis B, herpes simplex infection, HIF-1 signaling pathway, hippo signaling pathway, histidine metabolism, homologous recombination, HTLV-I infection, huntington's disease, hypertrophic cardiomyopathy (HCM), influenza A, insulin signaling pathway, legionellosis, Leishmaniasis, leukocyte transendothelial migration, lysine biosynthesis, lysosome, malaria, MAPK signaling pathway, meiosis—yeast, melanoma, metabolic pathways, metabolism of xenobiotics by cytochrome P450, microbial metabolism in diverse environments, microRNAs in cancer, mineral absorption, mismatch repair, natural killer cell mediated cytotoxicity, neuroactive ligand-receptor interaction, NF-kappa B signaling pathway, nitrogen metabolism, NOD-like receptor signaling pathway, non-alcoholic fatty liver disease (NAFLD), notch signaling pathway, olfactory transduction, oocyte meiosis, osteoclast differentiation, other glycan degradation, ovarian steroidogenesis, oxidative phosphorylation, p53 signaling pathway, pancreatic secretion, pantothenate and CoA biosynthesis, Parkinson's disease, pathways in cancer, penicillin and cephalosporin biosynthesis, peptidoglycan biosynthesis, peroxisome, pertussis, phagosome, phenylalanine metabolism, phenylalanine, tyrosine and tryptophan biosynthesis, phenylpropanoid biosynthesis, PI3K-Akt signaling pathway, plant-pathogen interaction, platelet activation, PPAR signaling pathway, prion diseases, proteasome, protein digestion and absorption, protein export, protein processing in endoplasmic reticulum, proteoglycans in cancer, purine metabolism, pyrimidine metabolism, pyruvate metabolism, Rap1 signaling pathway, Ras signaling pathway, regulation of actin cyto-skeleton, regulation of autophagy, renal cell carcinoma, renin-angiotensin system, retrograde endocannabinoid signaling, rheumatoid arthritis, RIG-I-like receptor signalling pathway, RNA degradation, RNA transport, salivary secretion, salmonella infection, serotonergic synapse, small cell lung cancer, spliceosome, Staphylococcus aureus infection, systemic lupus erythematosus, T cell receptor signaling pathway, taurine and hypotaurine metabolism, terpenoid backbone bio-synthesis, TGF-beta signaling pathway, TNF signaling pathway, Toll-like receptor signaling pathway, toxoplasmosis, transcriptional misregulation in cancer, tryptophan metabolism, tuberculosis, two-component system, type I diabetes mellitus, ubiquinone and other terpenoid-quinone biosynthesis, ubiquitin mediated proteolysis, vancomycin resistance, viral carcino-genesis, viral myocarditis, vitamin digestion, and absorption Wnt signaling pathway.

Target molecule fragments that can be used to measure peptidase inhibitors of interest include those in the MEROPS (an on-line database for peptidase inhibitors) which includes a total of ˜133,535 different sequences, where a family is a set of homologous peptidase inhibitors with a homology. The homology is shown by a significant similarity in amino acid sequence either to the type inhibitor of the family, or to another protein that has already been shown to be homologous to the type inhibitor. The reference organism for the family is shown ovomucoid inhibitor unit 3 (Meleagris gallopavo) aprotinin (Bos taurus), soybean Kunitz trypsin inhibitor (Glycine max), proteinase inhibitor B (Sagittaria sagittifolia), alpha-1-peptidase inhibitor (Homo sapiens), ascidian trypsin inhibitor (Halocynthia roretzi), ragi seed trypsin/alpha-amylase inhibitor (Eleusine coracana), trypsin inhibitor MCTI-1 (Momordica charantia), Bombyx subtilisin inhibitor (Bombyx mori), peptidase B inhibitor (Saccharomyces cerevisiae), marinostatin (Alteromonas sp.), ecotin (Escherichia coli), Bowman-Birk inhibitor unit 1 (Glycine max), eglin c (Hirudo medicinalis), hirudin (Hirudo medicinalis), antistasin inhibitor unit 1 (Haementeria officinalis), streptomyces subtilisin inhibitor (Streptomyces albogriseolus), secretory leukocyte peptidase inhibitor domain 2 (Homo sapiens), mustard trypsin inhibitor-2 (Sinapis alba), peptidase inhibitor LMPI inhibitor unit 1 (Locusta migratoria), potato peptidase inhibitor II inhibitor unit 1 (Solanum tuberosum), secretogranin V (Homo sapiens), BsuPI peptidase inhibitor (Bacillus subtilis), pinA Lon peptidase inhibitor (Enterobacteria phage T4), cystatin A (Homo sapiens), ovocystatin (Gallus gallus), metallopeptidase inhibitor (Bothrops jararaca), calpastatin inhibitor unit 1 (Homo sapiens), cytotoxic T-lymphocyte antigen-2 alpha (Mus musculus), equistatin inhibitor unit 1 (Actinia equina), survivin (Homo sapiens), aspin (Ascaris suum), saccharopepsin inhibitor (Saccharomyces cerevisiae), timp-1 (Homo sapiens), Streptomyces metallopeptidase inhibitor (Streptomyces nigrescens), potato metallocarboxypeptidase inhibitor (Solanum tuberosum), metallopeptidase inhibitor (Dickeya chrysanthemi), alpha-2-macroglobulin (Homo sapiens), chagasin (Leishmania major), oprin (Didelphis marsupialis), metallocarboxypeptidase A inhibitor (Ascaris suum), leech metallocarboxypeptidase inhibitor (Hirudo medicinalis), latexin (Homo sapiens), clitocypin (Lepista nebularis), proSAAS (Homo sapiens), baculovirus P35 caspase inhibitor (Spodoptera litura nucleopolyhedrovirus), p35 homologue (Amsacta moorei entomopoxvirus), serine carboxypeptidase Y inhibitor (Saccharomyces cerevisiae), tick anticoagulant peptide (Ornithodoros moubata), madanin 1 (Haemaphysalis longicornis), squash aspartic peptidase inhibitor (Cucumis sativus), staphostatin B (Staphylococcus aureus), staphostatin A (Staphylococcus aureus), triabin (Triatoma pallidipennis), pro-eosinophil major basic protein (Homo sapiens), thrombostasin (Haematobia irritans), Lentinus peptidase inhibitor (Lentinula edodes), bromein (Ananas comosus), tick carboxypeptidase inhibitor (Rhipicephalus bursa), streptopain inhibitor (Streptococcus pyogenes), falstatin (Plasmodium falciparum), chimadanin (Haemaphysalis longicornis), {Veronica} trypsin inhibitor (Veronica hederifolia), variegin (Amblyomma variegatum), bacteriophage lambda CIII protein (bacteriophage lambda), thrombin inhibitor (Glossina morsitans), anophelin (Anopheles albimanus), Aspergillus elastase inhibitor (Aspergillus fumigatus), AVR2 protein (Passalora fulva), IseA protein (Bacillus subtilis), toxostatin-1 (Toxoplasma gondii), AmFPI-1 (Antheraea mylitta), cvSI-2 (Crassostrea virginica), macrocypin 1 (Macrolepiota procera), HflC (Escherichia coli), oryctin (Oryctes rhinoceros), trypsin inhibitor (Mirabilis Jalapa), F1L protein (Vaccinia virus), NvCI carboxypeptidase inhibitor (Nerita versicolor), Sizzled protein (Xenopus laevis), EAPH2 protein (Staphylococcus aureus), and Bowman-Birk-like trypsin inhibitor (Odorrana versabilis). Rare molecule fragments can be used to measure synthetic inhibition of peptidase inhibitors. The aforementioned database also includes examples of thousands of different small molecule inhibitors that can mimic the inhibitory properties for any member of the above listed families.

Target molecule fragments include those of insulin, pro-insulin or c peptide generated by the following peptidases known to naturally act on insulin: archaelysin, duodenase, calpain-1, ammodytase subfamily M12B peptidases, ALE1 peptidase, CDF peptidase, cathepsin E, meprin alpha subunit, jerdohagin (Trimeresurus jerdonii), carboxypeptidase E, dibasic processing endopeptidase, yapsin-1, yapsin A, PCSK1 peptidase, aminopeptidase B, PCSK1 peptidase, PCSK2 peptidase, insulysin, matrix metallopeptidase-9 and others. These fragments include but are not limited to the following sequences: SEQ ID NO:1 MALWMRLLPLLALLALWGP, SEQ ID NO:2 MALWMRLLPL, SEQ ID NO:3 ALLALWGPD, SEQ ID NO:4 AAAFVN-QHLCGSHLVEALYLVCGERGFFYTPKTR, SEQ ID NO:5 PAAAFVNQHLCGSHLVEAL-YLVC, SEQ ID NO:6 PAAAFVNQHLCGS, SEQ ID NO:7 CGSHLVEALYLV, SEQ ID NO:8 VEALYLVC, SEQ ID NO:9 LVCGERGF, SEQ ID NO:10 FFYTPK, SEQ ID NO:11 REAEDLQVGQVELGGGPGAGSLQPLALEGSL, SEQ ID NO:12 REAEDLQVGQVE, SEQ ID NO:13 LGGGPGAG, SEQ ID NO:14 SLQPLALEGSL, SEQ ID NO:15 GIVEQCCTSICSL-YQLENYCN, SEQ ID NO:16 GIVEQCCTSICSLY, SEQ ID NO:17 QLENYCN, and SEQ ID NO:18 CSLYQLE, and variations within 75% of exact homology. Variations include natural and modified amino acids.

Target molecule fragments of insulin can be used to measure the peptidases acting on insulin based on formation of fragments. This includes the list of natural known peptidases and others added to the biological system. Additional rare molecule fragments of insulin can be used to measure inhibitors for peptidases acting on insulin based on the lack formation of fragments. These inhibitors include the c-terminal fragment of the Adiponectin Receptor, Bikunin, Uristatin and other known natural and synthetic inhibitors of archaelysin, duodenase, calpain-1, ammodytase subfamily M12B peptidases, ALE1 peptidase, CDF peptidase, cathepsin E, meprin alpha subunit, jerdohagin (Trimeresurus jerdonii), carboxypeptidase E, dibasic processing endopeptidase, yapsin-1, yapsin A, PCSK1 peptidase, aminopeptidase B, PCSK1 peptidase, PCSK2 peptidase, insulysin, and matrix metallopeptidase-9 listed in the inhibitor databases.

Target molecule fragments of bioactive therapeutic proteins and peptides can be used to measure the presence or absence thereof as an indication of therapeutic effectiveness, stability, usage, metabolism, action on biological pathways (such as actions with proteases, peptidase, enzymes, receptors or other biomolecules), action of inhibition of pathways and other interactions with biological systems. Examples include, but are not limited to, those listed in databases of approved therapeutic peptides and proteins, such as http://crdd.osdd.net/, as well as other databases of peptides and proteins for dietary supplements, probiotics, food safety, veterinary products, and cosmetics usage. The list of the approved peptide and protein therapies includes examples of bioactive proteins and peptides for use in cancer, metabolic disorders, hematological disorders, immunological disorders, genetic disorders, hormonal disorders, bone disorders, cardiac disorders, infectious disease, respiratory disorders, neurological disorders, adjunct therapy, eye disorders, and malabsorption disorder. Bioactive proteins and peptides include those used as anti-thrombins, fibrinolytic, enzymes, antineoplastic agents, hormones, fertility agents, immunosupressive agents, bone related agents, antidiabetic agents, and antibodies

### D. Formation of Target Variants

The target variants can be as a result of translation, or posttranslational modification by enzymatic or non-enzymatic modifications. Post-translational modification refers to the covalent modification of proteins during or after protein biosynthesis. Post-translational modification can be through enzymatic or non-enzymatic chemical reaction. Phosphorylation is a common mechanism for regulating the activity of enzymes and is the most common post-translational modification. Enzymes can be oxidoreductases, hydrolases, lyases, isomerases, ligases or transferases as known commonly in enzyme taxonomy databases, such as http://enzyme.expasy.org/or http://www.enzyme-database.org/, which have more than 6000 entries.

Common modifications of target variants include the addition of hydrophobic groups for membrane localization, addition of cofactors for enhanced enzymatic activity, diphthamide formation, hypusine formation, ethanolamine phosphoglycerol attachment, acylation, alkylation, amide bond formation such as amino acid addition or amidation, butyrylation gamma-carboxylation dependent on Vitamin K[15], glycosylation, the addition of a glycosyl group to either arginine, asparagine, cysteine, hydroxylysine, serine, threonine, tyrosine, or tryptophan resulting in a glycoprotein, malonylationhydroxylation, iodination, nucleotide addition such as ADP-ribosylation, phosphate ester (O-linked) or phosphoramidate (N-linked) formation such as phosphorylation or adenylylation, propionylation pyroglutamate formation, S-glutathionylation, S-nitrosylation S-sulfenylation (aka S-sulphenylation), succinylation or sulfation. Non-enzymatic modification include the attachment of sugars, carbamylation, carbonylation or intentional recombinate or synthetic conjugation such as biotinylation or addition of affinity agents, such as histidine oxidation, formation of disulfide bonds between cystine residues, or pegylation (addition of polyethylene oxide groups).

Common reagents for intentional fragmentation and formation of target variants such as peptides and proteins include peptidases or reagents know to react with peptides and proteins. The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

Intentional fragmentation can generate specific fragments based on predicted cleavage sites for proteases (also termed peptidases or proteinases) and chemicals known to react with peptide and protein sequences. Common peptidases and chemicals for intentional fragmentation include Arg-C, Asp-N, BNPS oNCS/urea, caspase, chymotrypsin (low specificity), Clostripain, CNBr, enterokinase, factor Xa, formic acid, Glu-C, granzyme B, HRV3C protease, hydroxylamine, iodobenzoic acid, Lys-C, Lys-N, mild acid hydrolysis, NBS, NTCB, elastase, pepsin A, prolyl endopeptidase, proteinase K, TEV protease, thermolysin, thrombin, and trypsin.

Common reagents for intentional inhibition of fragmentation include enzymes, peptidases, proteases, reductants, oxidants, chemical reactants, and chemical inhibitors for enzymes, peptidases, proteases including chemicals above listed.

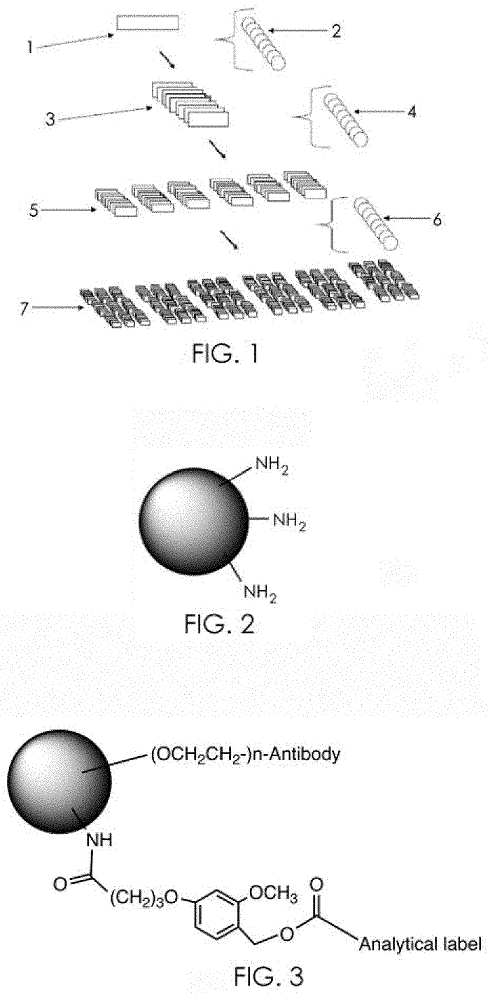

FIG. 1 is a schematic illustrating an example of the formation of target variants by fragmentation, addition, or binding. FIG. 1 shows an example of a group of proteases or peptidases acting on a single macromolecule such as a protein followed by additional reactions by a group of enzymes acting to generate a group of variations of the single protein. The original form of the analyte, such as the gene product 1 is acted on by a group of agents 2 (such as proteases) able to generate target variants by fragmentation which leads to fragments 3. These target variants 3 are acted on by a group of agents 4 able to generate additional target variants 5. These target variants may then be acted on by a group of agents 6 (such as proteins) able to generate target variants by binding to form additional target variants 7. After three cycles the number of target variants may easily be 100 or more.

### E. Target Cells

The target analytes may also comprise target cells. Target cells may include natural and synthetic cells. The cells may be found in biological samples that are suspected of containing the target cells, including both rare and non-rare cells. The samples may be biological samples or non-biological samples. Biological samples may be from a mammalian subject or a non-mammalian subject. Mammalian subjects may be humans or other animal species.

The disclosed materials and methods are useful with a wide variety of target cells and cell components. The target cells may comprise a population of cells, for example, a group of cells having an antigen or nucleic acid on their surface or inside the cell where the antigen is common to all of the cells of the group and where the antigen is specific for the group of cells. The term target cells also broadly encompasses cell components, such as biomarkers, which may be detected as analytes.

The target analytes may also comprise “target cellular molecules”, which refers to molecules that are contained in or bound to a cell, and which may or may not freely circulate in a sample. Such cellular molecules include biomolecules useful in medical diagnosis of diseases as above, and also include all molecules and uses previously described with respect to cell free molecules. The target cells may be, but are not limited to, malignant cells such as malignant neoplasms or cancer cells; circulating cells; endothelial cells (CD146); epithelial cells (CD326/EpCAM); mesochymal cells (VIM), bacterial cells, virus, skin cells, sex cells, fetal cells; immune cells (leukocytes such as basophil, granulocytes (CD66b) and eosinophil, lymphocytes such as B cells (CD19,CD20), T cells (CD3,CD4 CD8), plasma cells, and NK cells (CD56), macrophages/monocytes (CD14, CD33), dendritic cells (CD11c, CD123), Treg cells (and others), stem cells/precursor (CD34), other blood cells such as progenitor, blast, erythrocytes, thrombocytes, platelets (CD41, CD61, CD62) and immature cells; other cells from tissues such as liver, brain, pancreas, muscle, fat, lung, prostate, kidney, urinary tract, adipose, bone marrow, endometrium, gastrointestinal tract, heart, testis or other, for example.

The term “permeability” means the ability of a particle and molecule to diffuse through a barrier such as cellular walls or cellular membranes. In the case of molecule detection inside the cell, the diameter of the analyte detection particles must be small enough to allow the affinity agents to enter the cell. Alternatively, the linkage between the base particle and the affinity agent must be of sufficient length and possess sufficient permeability to allow the affinity agent access to the interior of the cell. The label particle may be coated with materials to increase “permeability” like collagenase, peptides, proteins, lipid, surfactants, and other chemicals known to increase particle permeability with respect to the cell.

As noted previously, the disclosed materials and methods may have particular advantage in the detection, isolation and/or analysis of target rare cells. By comparison, non-rare cells are those cells that are present in relatively large amounts when compared to the amount of rare cells in a sample. In some examples, the non-rare cells are at least about 10 times, or at least about 10² times, or at least about 10³ times, or at least about 10⁴ times, or at least about 10⁵ times, or at least about 10⁶ times, or at least about 10⁷ times, or at least about 10⁸ times greater than the amount of the rare cells in the total cell population in a sample suspected of containing non-rare cells and rare cells. The non-rare cells may be, but are not limited to, white blood cells, platelets, and/or red blood cells, for example.

The term “rare cell marker” includes, but is not limited to, cancer cell type biomarkers, cancer bio markers, chemo resistance biomarkers, metastatic potential biomarkers, and cell typing markers. A cluster of differentiation (cluster of designation or classification determinant, often abbreviated as CD) is a protocol used for the identification and investigation of cell surface molecules providing targets for immunophenotyping of cells. Cancer cell type biomarkers include, by way of illustration and not limitation, cytokeratins (CK) (CK1, CK2, CK3, CK4, CK5, CK6, CK7, CK8 and CK9, CK10, CK12, CK 13, CK14, CK16, CK17, CK18, CK19 and CK2), epithelial cell adhesion molecule (EpCAM), N-cadherin, E-cadherin and vimentin, for example. Oncoproteins and oncogenes with likely therapeutic relevance due to mutations include, but are not limited to, WAF, BAX-1, PDGF, JAGGED 1, NOTCH, VEGF, VEGHR, CALX, MIB1, MDM, PR, ER, SELS, SEMI, PI3K, AKT2, TWIST1, EML-4, DRAFF, C-MET, ABL1, EGFR, GNAS, MLH1, RET, MEK1, AKT1, ERBB2, HER2, HNF1A, MPL, SMAD4, ALK, ERBB4, HRAS, NOTCH1, SMARCB1, APC, FBXW7, IDH1, NPM1, SMO, ATM, FGFR1, JAK2, NRAS, SRC, BRAF, FGFR2, JAK3, RA, STK11, CDH1, FGFR3, KDR, PIK3CA, TP53, CDKN2A, FLT3, KIT, PTEN, VHL, CSF1R, GNA11, KRAS, PTPN11, DDR2, CTNNB1, GNAQ MET, RB1, AKT1, BRAF, DDR2, MEK1, NRAS, FGFR1, and ROS1, for example.

In certain embodiments, the target cells may be endothelial cells which are detected using markers, by way of illustration and not limitation, CD136, CD105/Endoglin, CD144/VE-cadherin, CD145, CD34, Cd41 CD136, CD34, CD90, CD31/PECAM-1, ESAM, VEGFR2/Fik-1, Tie-2, CD202b/TEK, CD56/NCAM, CD73/VAP-2, claudin 5, ZO-1, and vimentin. Metastatic potential biomarkers include, but are limited to, urokinase plasminogen activator (uPA), tissue plasminogen activator (tPA), C terminal fragment of adiponectin receptor (Adiponectin Receptor C Terminal Fragment or Adiponectin CTF), kinases (AKT-PIK3, MAPK), vascular adhesion molecules (e.g., ICAM, VCAM, E-selectin), cytokine signaling (TNF-α, IL-1, IL-6), reactive oxidative species (ROS), protease-activated receptors (PARs), metalloproteinases (TIMP), transforming growth factor (TGF), vascular endothelial growth factor (VEGF), endothelial hyaluronan receptor 1 (LYVE-1), hypoxia-inducible factor (HIF), growth hormone (GH), insulin-like growth factors (IGF), epidermal growth factor (EGF), placental growth factor (PDF), hepatocyte growth factor (HGF), nerve growth factor (NGF), platelet-derived growth factor (PDGF), growth differentiation factors (GDF), VEGF receptor (soluble Flt-1), microRNA (MiR-141), Cadherins (VE, N, E), S100 Ig-CTF nuclear receptors (e.g., PPARα), plasminogen activator inhibitor (PAI-1), CD95, serine proteases (e.g., plasmin and ADAM, for example); serine protease inhibitors (e.g., Bikunin); matrix metalloproteinases (e.g., MMP9); matrix metalloproteinase inhibitors (e.g., TIMP-1); and oxidative damage of DNA.

Chemoresistance biomarkers include, by way of illustration and not limitation, PL2L piwi like, 5T4, ADLH, β-integrin, α-6-integrin, c-kit, c-met, LIF-R, chemokines (e.g., CXCR7, CCR7, CXCR4), ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24.

Target molecules from cells may be from any organism, which includes, but is not limited to, pathogens such as bacteria, virus, fungus, and protozoa; malignant cells such as malignant neoplasms or cancer cells; circulating endothelial cells; circulating tumor cells; circulating cancer stem cells; circulating cancer mesenchymal cells; circulating epithelial cells; fetal cells; immune cells (B cells, T cells, macrophages, NK cells, monocytes); and stem cells; for example. In some examples of methods in accordance with the principles described herein, the sample to be tested is a blood sample from a mammal such as, but not limited to, a human subject.

Target cells of interest may be immune cells and include, but are not limited to, markers for white blood cells (WBC), Tregs (regulatory T cells), B cell, T cells, macrophages, monocytes, antigen presenting cells (APC), dendritic cells, eosinophils, and granulocytes. For example, markers such as, but not limited to, CD3, CD4, CD8, CD11c, CD14, CD15, CD16, CD19, CD20, CD31, CD33, CD45, CD52, CD56, CD 61, CD66b, CD123, CTLA-4, immunoglobulin, protein receptors and cytokine receptors and other CD markers that are present on white blood cells can be used to indicate that a cell is not a rare cell of interest.

In particular non-limiting examples, white blood cell markers include CD45 antigen (also known as protein tyrosine phosphatase receptor type C or PTPRC) and originally called leukocyte common antigen is useful in detecting all white blood cells. Additionally, CD45 can be used to differentiate different types of white blood cells that might be considered rare cells. For example, granulocytes are indicated by CD45+, CD15+, or CD16+, or CD66b+; monocytes are indicated by CD45+, CD14+; T lymphocytes are indicated by CD45+, CD3+; T helper cells are indicated by CD45+, CD3+, CD4+; cytotoxic T cells are indicated by CD45+, CD3+, CDS+; B-lymphocytes are indicated by CD45+, CD19+ or CD45+, CD20+; thrombocytes are indicated by CD45+, CD61+; and natural killer cells are indicated by CD16+, CD56+, and CD3−. Furthermore, two commonly used CD molecules, namely, CD4 and CD8, are, in general, used as markers for helper and cytotoxic T cells, respectively. These molecules are defined in combination with CD3+, as some other leukocytes also express these CD molecules (some macrophages express low levels of CD4; dendritic cells express high levels of CD11c, and CD123. These examples are not inclusive of all markers and are for example only.

In some cases, target analytes comprise fragments of lymphocytes, including proteins and peptides produced as part of lymphocytes such as immunoglobulin chains, major histocompatibility complex (MHC) molecules, T cell receptors, antigenic peptides, cytokines, chemokines and their receptors (e.g, Interluekins, C-X-C chemokine receptors, etc), programmed death-ligand and receptors (Fas, PDL1, and others) and other proteins and peptides that are either parts of the lymphocytes or bind to the lymphocytes.

In other cases, the target cells may be stem cells, and include, but are not limited to, the molecule fragments of stem marker cells including, PL2L piwi like, 5T4, ADLH, β-integrin, α6 integrin, c-kit, c-met, LIF-R, CXCR4, ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24. Stem cell markers include common pluripotency markers like FoxD3, E-Ras, Sall4, Stat3, SUZ12, TCF3, TRA-1-60, CDX2, DDX4, Miwi, Mill GCNF, Oct4, Klf4, Sox2, c-Myc, TIF 1 [symbol] Piwil, nestin, integrin, notch, AML, GATA, Esrrb, Nr5a2, C/EBPα, Lin28, Nanog, insulin, neuroD, adiponectin, apdiponectin receptor, FABP4, PPAR, and KLF4 and the like.

In other cases the rare cell may be a pathogen, bacteria, or virus or group thereof which includes, but is not limited to, gram-positive bacteria (e.g., Enterococcus sp. Group B streptococcus, Coagulase-negative staphylococcus sp. Streptococcus viridans, Staphylococcus aureus and saprophyicus, Lactobacillus and resistant strains thereof, for example); yeasts including, but not limited to, Candida albicans, for example; gram-negative bacteria such as, but not limited to, Escherichia coli, Klebsiella pneumoniae, Citrobacter koseri, Citrobacter freundii, Klebsiella oxytoca, Morganella morganii, Pseudomonas aeruginosa, Proteus mirabilis, Serratia marcescens, Diphtheroids (gnb), Rosebura, Eubacterium hallii, Faecalibacterium prauznitzli, Lactobacillus gasseria, Streptococcus mutans, Bacteroides thetaiotaomicron, Prevotella Intermedia, Porphyromonas gingivalis Eubacterium rectale Lactobacillus amylovorus, Bacillus subtilis, Bifidobacterium longum Eubacterium rectale, E. eligens, E. dolichum, B. thetaiotaomicron, E. rectale, Actinobacteria, Proteobacteria, B. thetaiotaomicron, Bacteroides Eubacterium dolichum, Vulgatus, B. fragilis, bacterial phyla such as Firmicuties (Clostridia, Bacilli, Mollicutes), Fusobacteria, Actinobacteria, Cyanobacteria, Bacteroidetes, Archaea, Proteobacteria, and resistant strains thereof, for example; viruses such as, but not limited to, HIV, HPV, Flu, and MRSA, for example; and sexually transmitted diseases. In the case of detecting rare cell pathogens, a collection particle is added that comprises an affinity agent, which binds to the rare cell pathogen population. Additionally, for each population of cellular rare molecules on the pathogen, a reagent is added that comprises an affinity agent for the cellular rare molecule, which binds to the cellular rare molecules in the population.

### F. Target Cell Samples

The target cell sample may be any that contains cells such as, for example, non-target cells and target cells. Target molecules may be detected from the target cells. The target molecules from cells may be from any organism, and are not limited to, pathogens such as bacteria, virus, fungus, and protozoa; malignant cells such as malignant neoplasms or cancer cells; circulating endothelial cells; circulating tumor cells; circulating cancer stem cells; circulating cancer mesochymal cells; circulating epithelial cells; fetal cells; immune cells (B cells, T cells, macrophages, NK cells, monocytes); and stem cells; for example. In other examples of methods in accordance with the invention described herein, the sample to be tested is a fluid sample from an organism such as, but not limited to, a plant or animal subject, for example. In some examples of methods in accordance with the principles described herein, the sample to be tested is a sample from an organism such as, but not limited to, a mammalian subject, for example. Target cells with target molecules may be from a tissue of mammal, for example, lung, bronchus, colon, rectum, pancreas, prostate, breast, liver, bile duct, bladder, ovary, brain, central nervous system, kidney, pelvis, uterine corpus, oral cavity or pharynx or cancers.

### II. Particles

The analyte detection particles are formed using base particles to which suitable linker arms are bound. The base particles may be any particulate material which is attachable to a label, collection particle and/or affinity agent by a linker arm. The linker arms are organic components which are able to couple the base particles with the labels, collection particles and/or affinity agents.

The composition of the base particle may be organic or inorganic, magnetic or non-magnetic. Organic polymers include, by way of illustration and not limitation, nitrocellulose, cellulose acetate, poly(vinyl chloride), polyacrylamide, polyacrylate, polyethylene, polypropylene, poly(4-methylbutene), polystyrene, poly(methyl methacrylate), poly(hydroxyethyl methacrylate), poly(styrene/divinyl-5 benzene), poly(styrene/acrylate), poly(ethylene terephthalate), dendrimer, melamine resin, nylon, poly(vinyl butyrate), for example, either used by themselves or in conjunction with other materials including latex. The base particles may also be composed of carbon (e.g., carbon nanotubes), metal (e.g., gold, silver, and iron, including metal oxides thereof), colloids, dendrimers, dendrons, and liposomes, for example. In some examples, the particles can be silica.

The base particles are functionalized and provide sites for binding linker arms to the base particles. For example, base particles may exhibit or be modified to exhibit free carboxylic acid, amine or tosyl groups, by way of example and not limitation. In some examples, base particles can be mesoporous and include labels within pores. (29/28-30/12) Various functional components are known in the art. For example, in one aspect, the particles are aminated particles, as shown in FIG. 2.

Various sizes are useful in label detection approaches, particularly to allow for special retention techniques. The sizes of the base particles relate to their intended use. In one aspect, the particles are nanoparticles, defined as particles having a nominal size of 300 nm to 10 nm. In an alternative approach, the particles are nanoparticles having a nominal size of 200 nm to 20 nm.

### III. Analytical Labels

The terms “analytical label” or “label” refer to a chemical entity (organic or inorganic) which is capable of generating a detectable signal, detected for example by optical, mass spectroscopy, or electrochemical means. The label may be detected directly on a substrate, on a porous matrix, or in a liquid. Analytical labels are molecules, metals, ions, atoms, or electrons that are detectable using an analytical method to yield information about the presence and amounts of the target analytes in a sample.

The analytical labels are attached to, and may be releasable from, the analyte detection particles. The analytical labels may therefore be used to identify target analytes coupled with the analyte detection particles. The analytical labels may be measured in a conventional manner with an internal standard as a calibrator which is structurally similar or identical to the analytical label.

### A. Mass Labels

In one aspect, the analyte detection particles and related methods and systems are directed to using mass labels as the analytical labels for detection of the target analytes. The term “mass label” refers to a molecule having a unique mass spectral signature that corresponds to, and is used to, determine a presence and/or amount of the target analytes. The mass label can additionally be fluorescent, chemiluminescent or electrochemical in nature. The mass labels may, in some instances, be peptides with unique fragmentation patterns. Charges can be permanent or temporary charges.

Examples of peptides, which may function as mass labels, include, by way of illustration and not limitation, peptides that contain two or more of histidine, lysine, phenylalanine, leucine, alanine, methionine, asparagine, glutamine, aspartic acid, glutamic acid, tryptophan, proline, valine, tyrosine, glycine, threonine, serine, arginine, cysteine and isoleucine and derivatives thereof. In some examples, the peptides have a molecular weight of about 100 to about 3,000 Da and may contain 3 to 30 amino acids, either naturally occurring or synthetic. The number of amino acids in the peptide is determined by, for example, the nature of the mass spectroscopy technique employed. For example, when using MALDI for detection, the peptide can have a mass in the range of about 600 to about 3,000 and is constructed of about 5 to about 30 amino acids. Alternatively, when using electrospray ionization for mass spectrometric analysis, the peptide has a mass in the range of about 100 to about 1,000 and is constructed of 1 to 10 amino acids or derivatives thereof, for example. In some examples, the number of amino acids in the peptide label may, for example, be from 1 to 30. The mass labels can include ionized groups, such as quaternary ammonium salts like carnitine, betaine, lysine salt, arginine salts, guanidine salts and their derivatives; quaternary aromatic ammonium salts like imidazole, pyrrole, histidine, quinoline, pyridine, indole, purine pyrimidine, and the like; tetra alkyl ammonium ions, tri alkyl sulfonium ions, tetra alkyl phosphonium ions and other examples.

The use of peptides as mass labels has several advantages, which include, but are not limited to, the following: 1) relative ease of conjugation to proteins, antibodies, particles and other biochemical entities; 2) relative ease with which the mass can be altered to allow many different masses thus providing for multiplexed assay formats and standards; 3) the ability to fragment reproducibly into detectable and predictable masses and/or 4) adjustability of the molecular weight for optimal performance with the mass spectrometer used for detection. For conjugation, the peptides can have a terminal cysteine that is employed in the conjugation.

In order to aid in efficient ionization, the peptides may have permanently charged, or readily ionizable, amine groups. In some examples, the peptides have N-terminal free amine and/or C-terminal free acid groups. In some examples, the peptides incorporate one or more stable isotopes or are derivatized with one or more stable isotopes. The peptides may be conjugated to a small molecule such as, for example, biotin or fluorescein, for binding to a corresponding binding partner for the small molecule, which for example may be streptavidin or antibody for fluorescein.

### B. Optical Labels

The phrase “optical labels” refers to molecules that allow for specific detection by optical means, such as: a chemiluminescent label such as luminol, isoluminol, acridinium esters, adamantyl 1, 2-dioxetane aryl phosphate, acridinium sulfonamides metals derivatives or others as known in the field; a fluorescent label such as fluorescein, lanthanide metals, Hoechst 33258, R-phycocyanin, B-phycoerythrin, R-phycoerythrin, rhodamine, DyLight Dyes™, Texas red, metals FITC, rhodamine compounds, phycoerythrin, phycocyanin, allophycocyanin, o-phthaldehyde, fluorescent rare earth chelates, amino-coumarins, umbelliferones, oxazines, acridones, perylenes, indacines such as, e.g., 4,4-difluoro-4-bora-3a,4a-diaza-s-indacene and variants thereof, 9,10-bis-phenylethynylanthracene, squaraine dyes, fluorescamine, or others as known to researchers in the field (see http://www.fluorophores.org/); or a chromogenic label such as tetramethylbenzidine (TMB), particles, metals or others as known in the field. Optical analytical labels are detectable by optical methods such as by microscope, camera, optical reader, colorimeter, fluorometer, luminometer, reflectrometer, and others.

Obtaining reproducibility in regards to the amounts of label and collection particles retained after separation and isolation is important for rare molecular analysis. Additionally, knowledge of the amounts of particles which enter a cell is important to maximize the amount of specific binding. Knowing the amount of particles which remain after washing is important to minimize the amount of non-selective binding. In order to make these determinations, it is helpful if the particles include “optical labels” which include fluorescent, colored, or chemiluminescence labels. Therefore, the presence of label particles can be measured by virtue of the presence of an optical label. The optical labels can be measured by microscopy and results compared for samples containing and lacking analyte.

### C. Electrochemical Labels

The phrase “electrochemical labels” refers to potentiometric, capacitive and redox active compounds such as: metals such as Pt, Ag, Pd, Au and many others; particles such as gold sols, graphene oxides and many others; electron transport molecules such as ferrocene, ferrocyanide, Os(VI)bipy and many others; electrochemical redox active molecules such as aromatic alcohols and amines such as 4-aminophenyl phosphate, 2-naphthol, para-nitrophenol phosphate; thiols or disulfides such as those on aromatics, aliphatics, amino acids, peptides and proteins; aromatic heterocyclic containing non-carbon ring atoms, such as oxygen, nitrogen, or sulfur such as imidazoles, indoles, quinolones, thiazole, benzofuran and many others. Electrochemical analytical labels are detectable by impedance, capacitance, amperometry, electrochemical impedance spectroscopy and other measurement.

### D. Label Precursors

A “mass label precursor” is any molecule, particle, or combination of both from which a mass label may be formed or generated. The mass label precursor may, through the action of an alteration agent, be converted to a mass label by cleavage, by reaction with a moiety, by derivatization, or by addition or by subtraction of molecules, charges or atoms, for example, or a combination of two or more of the above. In some examples target analytes are retained on the porous matrix or collection particle and reacted to generate an analytical label from the porous matrix or collection particle.

In some examples, mass label precursors are used which comprise molecules whose mass can be varied by substitution and/or chain size. The nature of the mass label precursors is dependent on one or more of the nature of the mass label, the nature of the mass spectroscopy method employed, the nature of the mass spectroscopy detector employed, the nature of the target rare molecules, the nature of the affinity agent, the nature of any immunoassay employed, the nature of the sample, the nature of any buffer employed, and/or the nature of the separation.

In another example, a derivatization agent is employed to generate a mass label from a mass label precursor. For example, dinitrophenyl and other nitrophenyl derivatives may be formed from a mass label precursor. Other examples include, by way of illustration and not limitation, esterification, acylation, silylation, protective alkylation, derivatization by ketone-base condensations such as Schiff bases, cyclization, formation of fluorescent derivatives, and inorganic anions. The derivatization reactions can occur prior to mass spectroscopy analysis, after an affinity reaction or be used to generate mass label precursors which are conjugated to affinity reagents.

In some examples, the mass label precursor can include one or more isotopes such as, but not limited to, ²H, ¹³C, and ¹⁸O, for example, which remain in the mass label that is derived from the mass label precursor. The mass label can be detected based on a mass spectroscopic signature. In some examples, the mass label precursor is one that has a relatively high potential to cause a bond cleavage such as, but not limited to, alkylated amines, acetals, primary amines and amides, for example.

The mass labels produced from the mass label precursors are molecules of defined molecular weight and structure. The mass labels should be detectable by the mass spectroscopy detector and should not be subject to background interference by the sample or analysis liquid. Examples, by way of illustration and not limitation, of mass label precursors for use in methods in accordance with the principles described herein to produce mass labels include, by way of illustration and not limitation, polypeptides, organic and inorganic polymers, fatty acids, carbohydrates, cyclic hydrocarbons, aliphatic hydrocarbons, aromatic hydrocarbons, organic carboxylic acids, organic amines, nucleic acids, organic alcohols (e.g., alkyl alcohols, acyl alcohols, phenols, polyols (e.g., glycols), thiols, epoxides, primary, secondary and tertiary amines, indoles, tertiary and quaternary ammonium compounds, amino alcohols, amino thiols, phenolic amines, indole carboxylic acids, phenolic acids, vinylogous acid, carboxylic acid esters, phosphate esters, carboxylic acid amides, carboxylic acids from polyamides and polyesters, hydrazone, oxime, trimethylsilyl enol ether, acetal, ketal, carbamates, guanidines, isocyanates, sulfonic acids, sulfonamides, sulfonyl sulfates esters, monoglycerides, glycerol ethers, sphingosine bases, ceramines, cerebrosides, steroids, prostaglandins, carbohydrates, nucleosides and therapeutic drugs, for example.

A polypeptide mass label is any mass label that is composed of repeating units or sequences of amino acids. In the case of a polypeptide mass label, the identity and/or number of amino acid subunits can be adjusted to yield a mass label displaying a mass spectroscopic signature or peak not subject to background interference. Furthermore, mass spectrometry analytical labels may be produced from analytical label precursors having unique mass spectroscopic signatures, which are not present in the sample tested. The polypeptide analytical label precursors can include additional amino acids or derivatized amino acids, which allows for multiplexed measurements to obtain more than one result in a single analysis. Examples of polypeptide mass label precursors include, but are not limited to, polyglycine, polyalanine, poly-serine, polythreonine, polycysteine, polyvaline, polyleucine, polyisoleucine, polymethionine, polyproline, polyphenylalanine, polytyrosine, polytryptophan, polyaspartic acid, polyglutamic acid, polyasparagine, polyglutamine, polyhistidine, polylysine and polyarginine, for example. In some examples, polypeptides are modified by catalysis. For example, by way of illustration and not limitation, phenol and aromatic amines can be added to polythreonine using a peroxidase enzyme as a catalyst. In another example, by way of illustration and not limitation, electrons can be transferred to aromatic amines using peroxidase enzyme as a catalyst. In another example, by way of illustration and not limitation, phosphates can be removed from organic phosphates using phosphatases as a catalyst.

### E. Amplification

The number of labels and/or affinity agents associated with a given analyte detection particle may depend on a variety of factors, including the nature and size of the base particle, and the number and type of functional groups on the base particle. Additional factors include the nature and size of the label, the affinity agents and the linker arms. The ratio of labels and/or affinity agents on a single particle may be 108 to 1, 106 to 1, or 105 to 1, or 104 to 1, or 103 to 1, or 102 to 1, or 10 to 1, for example.

Some examples in accordance with the invention described herein are directed to methods of measuring an analyte which use particle amplification of analytical labels through attachment of multiple analytical labels in an analyte detection particle. Multiple analytical labels on a single analyte detection particle allow amplification as every analyte detection particle can include numerous labels. In some examples, directed to methods of amplification, there are multiple analytical labels attached to analyte detection particles with affinity agents. An analyte detection particle can include 1 to about 10⁸ labels, or about 10 to about 10⁴ labels, or about 10³ to about 10⁵ labels, or about 10⁴ to about 10⁸ labels, or about 10⁶ to about 10⁸ labels, for example. In other examples, additional affinity agents can be linked to collection particles and the collection particles are used to isolate label particles with affinity agents on to a porous matrix or magnet.

### IV. Affinity Agents

The analyte detection particles include affinity agents to couple with the target analytes. The affinity agents have an “affinity” for the target analytes. As used herein, the term “affinity” refers to the ability to specifically couple with a select target analyte. Selective binding involves the specific recognition of a target molecule compared to substantially less recognition of other molecules. The coupling may be through non-covalent binding such as a specific ionic binding, hydrophobic binding, pocket binding and the like. In contrast, “non-specific binding” may result from several factors including hydrophobic or electrostatic interactions between molecules that are general and not specific to any particular molecule in a class of similar molecules. The affinity agents may be attached to the analyte detection particles by linker arms including cleavable or non-cleavable bonds depending on the intended detection method. The coupling may be by any manner of attachment provided the coupling is sustained to the extent required for subsequent detection steps.

The affinity agents are coupled with the target analytes in order to associate the target analytes with the labels. The labels may be removed from the analyte detection particles while the target analytes remain coupled with the analyte detection particles, or the target analytes may be cleaved from the analyte detection particles while the labels remain coupled. In one aspect, for example, the labels are cleaved and collected for further evaluation, e.g., to determine the amount or concentration of the target analytes in the sample. The target analytes may then be cleaved from the analyte detection particles and further processed, such as by visual examination of target cells.

An affinity agent can be an immunoglobulin, protein, peptide, metal, carbohydrate, metal chelator, nucleic acid, aptamer, xeno-nucleic acid, xeno-peptide, antigen which binds to an immunoglobulin analyte, or other molecule capable of binding selectively to a particular molecule. The affinity agents which are immunoglobulins may include complete antibodies or fragments thereof, including the various classes and isotypes, such as IgA, IgD, IgE, IgG1, IgG2a, IgG2b and IgG3, IgM, etc. Fragments thereof may include Fab, Fv and F(ab′)2, and Fab′, for example. In addition, aggregates, polymers, and conjugates of immunoglobulins or their fragments can be used where appropriate so long as binding affinity for a particular molecule is maintained.

Antibodies are specific for target molecules and can be monoclonal or polyclonal. Such antibodies can be prepared by techniques that are well known in the art such as immunization of a host and collection of sera (polyclonal) or by preparing continuous hybrid cell lines and collecting the secreted protein (monoclonal) or by cloning and expressing nucleotide sequences or mutagenized versions thereof coding at least for the amino acid sequences required for specific binding of natural antibodies. Polyclonal antibodies and monoclonal antibodies may be prepared by techniques that are well known in the art. For example, in one approach monoclonal antibodies are obtained by somatic cell hybridization techniques. Monoclonal antibodies may be produced according to the standard techniques of Köhler and Milstein, Nature 265:495-497, 1975. Reviews of monoclonal antibody techniques are found in Lymphocyte Hybridomas, ed. Melchers, et al. Springer-Verlag (New York 1978), Nature 266: 495 (1977), Science 208: 692 (1980), and Methods of Enzymology 73 (Part B): 3-46 (1981). In general, monoclonal antibodies can be purified by known techniques such as, but not limited to, chromatography, e.g., DEAE chromatography, ABx chromatography, and HPLC chromatography; and filtration, for example.

An affinity agent can additionally be a “cell affinity agent” capable of binding selectively to a target molecule which is used for typing a target cell or measuring a biological intracellular process of a target cell. These affinity agents can be immunoglobulins that specifically recognize and bind to an antigen associated with a particular cell type and whereby the antigen is a component of the cell. The cell affinity agent is capable of being absorbed into or onto the cell. Selective cell binding typically involves binding (between molecules) that is relatively dependent on specific structures of the binding pair (affinity agent target molecule). Many other suitable affinity agents would be well known to those of ordinary skill in the relevant art.

### V. Linker Arms

Linker arms are provided which serve various purposes for the analytical detection and analyte collection particles. The labels, collection particles and affinity agents are coupled with the base particles by way of linker arms. The linker arms are attached to the functionalized base particles. In the synthesis and use of the analyte detection particles, the linker molecules are at some point coupled at one end to the base particles and at the other end to the labels, collection particles, or affinity agents. The linker arms are thus formed using linker molecules that include functional groups suited to provide these attachments. These attachments may use a variety of complementary functional groups that react together to join these components. For example, in one embodiment the linker arms are coupled with the base particles by way of surface amine groups.

The linker arms are generally cleavable under select conditions. The linker arms may be cleaved by breaking the bond binding the linker arms to the coupled labels, collection particles, and/or affinity agents. Cleavage of the linker arm results in the separation of the base particle from the moiety coupled by the linker arm. However, the linker arms need not have such cleavable bonds in certain embodiments. For example, if the labels of an analyte detection particle are to be removed and tested, and no further processing is intended for the target analytes, then the affinity linker arms are not required to be cleavable.

### A. Binding the Base Particles

One end of the linker arm is bonded to the base particle. As used herein, the term “bond” may include any type of coupling which functions as required for the indicated purpose. The bond may be of any type, including covalent or ionic for example. A wide variety of linkages as known in the art may be used for binding the linker arms to the base particles. For example, carboxylic acid, hydroxyl, sulfide and amine groups generally allow for suitable binding of the linker arms to the base particles. Other bonds may include esters, amides and disulfide bonds that bind with the base particles, and other well-known bonds may instead be used. As a further example, the bonds may comprise any suitable for the attachment of PEG groups, such as amine-reactive N-hydroxysuccinimde (NHS) esters, imido esters, difluro nitrobenzene, NHS-haloacetyl, NHS maleimide and NHS pyridyldithiol groups.

### B. Cleavable Bonds

A linker arm may form a cleavable bond with the attached label, affinity agent, and/or target analyte. The term “cleavable bond”, as used herein, refers to a bond which may be cleaved under “cleavage conditions”. Cleavage conditions for a given linker arm are those conditions under which a cleavable bond between the linker arm and the coupled moiety is cleaved. The cleavage conditions used herein do not cleave the bonds between the base particles and the linker arms. It is an aspect that cleavage conditions are not such as to materially diminish the usefulness of the labels and/or target analytes for the collection, detection and/or analysis contemplated herein. The terms “viable” and “viable for analysis” refer to the label and/or target analyte being maintained in a condition suitable for subsequent analysis by the intended detection methods.

By way of example, label linker arms couple labels to the base particles of the analyte detection particles. The labels need to be retained in a viable condition during coupling of the analyte detection particles with the target analytes to form analyte complexes, as well as during collection of the analyte complexes. At this point, the labels may be detected while still a part of the analyte complexes and the labels must be viable during detection of the labels. Alternatively, the labels may be cleaved from the analyte complexes prior to detection, and the labels must remain viable under the label cleavage conditions and during subsequent detection. In summary, the labels must remain viable through to the time of their detection.

As another example, the target analytes must also remain viable during formation of the analyte complexes, and during subsequent collection of the analyte complexes. At this point, the target analytes may be processed for further analysis, either as part of the analyte complexes, or following cleavage from the analyte complexes. If cleaved prior to further processing, the target analytes must remain viable under the affinity cleavage conditions and during subsequent detection processing. In summary, the target analytes must remain viable through to the time of their detection.

### C. The Cleavable C—O Bond

The label, affinity agent and/or target analyte as disclosed herein are coupled with the linker arms by cleavable ether (“C—O”) bonds. The ether bonds form between the linker arms and the labels, affinity agents and/or target analytes. By way of example, the linker molecules used to form the cleavable bonds of the analyte detection particles may have a structure including the elements shown in Structure I:

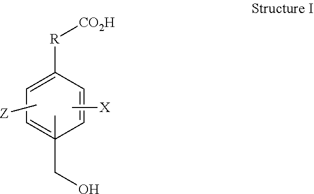

In Structure I, R is a non-interfering organic group comprising alkyls, polyamides, polypeptides polyethers and other polymeric chains. Key polymeric chains include alkyl and polyether chains, with PEG being a commonly used polymer. Further examples include groups normally consisting of hydrogen, carbon, oxygen, sulfur, nitrogen, and phosphorous, usually hydrogen, carbon and oxygen, and can include repeating alkyl, aryl, aralkyl, hydroxyl, alkoxy, aryloxy, or aralkoxy groups.

The “X” groups are non-interfering organic groups, meaning that the X substituents do not interfere with the functioning of the analyte detection and collection particles. The X groups may be utilized to establish the cleavage conditions for the ether bond formed with the label, collection particle and/or target analyte. There may be multiple X groups, which may be the same or different, and in particular may be hydrogen, or may be an electron donating or electron withdrawing group. The X group(s) can be selectively positioned relative to the CH2OH and COOH groups on the benzene ring. Selection and positioning of the X groups to achieve useful cleavage conditions is determinable by persons of ordinary skill in the art without undue experimentation.

Other non-interfering constituents, as represented by “Z”, may also be present. These may be any other non-interfering moiety used for various purposes, such as to facilitate synthesis of the analyte detection and/or collection particles.

This linker molecule is an aromatic compound comprising a benzene ring including an appended carboxylic acid (COOH) group and an appended hydroxyl (CH₂OH) group. The COOH group is preferably at the para or ortho positions relative to the CH₂OH group, more preferably at the para position. The OH moiety of the carboxylic acid group binds at the amine group of the base particle.

The coupling with the label, affinity agent and/or target analyte is an ether bond through the appended hydroxyl (CH₂OH) group of the linker arm. The base particle is attached to the linker arm through the appended carboxylic acid (COOH) group. This exemplary linker molecule may be used to couple with the base particles to provide a Structure II:

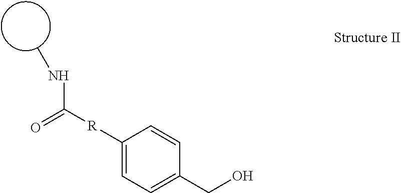

The oxygen in the CH₂OH group of the linker molecule forms an ether bond at the OH moiety of a carboxylic acid group on the label, affinity agent or target analyte, as shown in Structure III. It is this ether bond that is the cleavable bond for the linker arms that require cleavage. The linker molecule remains attached to the base particle after the C—O bond cleaves.

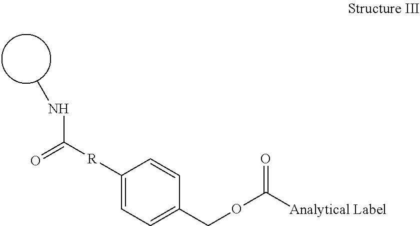

A representative linker molecule used in the examples herein is 4-(4-hydroxymethyl-3-methoxyphenoxy)butyric acid (HMPB). HMPB is known in the art to have the following structure:

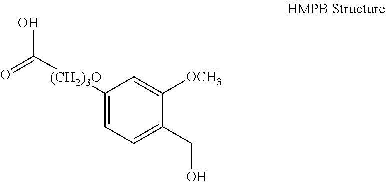

HMPB thus has the Structure I in which R is —O—(CH₂)₃— and there is additionally a group CH₃O— on the benzene ring at a position ortho to the CH₂OH group. As shown hereafter, the use of HMPB as the linker molecule yields a linker molecule coupled with a base particle as shown in Structure IV, and also as shown in Step 1.2.

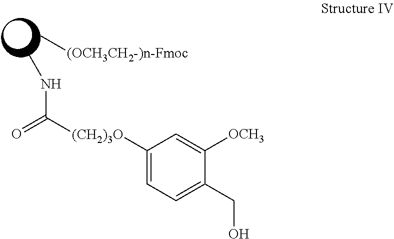

Similarly, HMPB forms an analyte detection particle as shown in Structure V, and also as shown in Step 2.2.

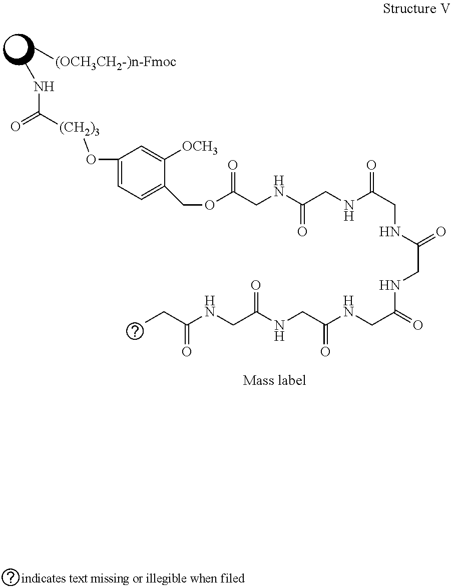

It will be appreciated that variations in the linker molecule structure are also useful. For example, the CH₂OH group is shown in the para position relative to the COOH group, but may instead be positioned at any other position on the ring. In another aspect, the linker molecule may also have the following expanded Structure VI:

In FIG. 3 there is shown an analyte detection particle including both a label and an affinity agent attached to a nanoparticle. The drawing shows the attachment of the linker arms to the nanoparticle, and also to the label and affinity agent.

### D. Label Linker Arms

The label linker arms are coupled at one end to the base particle and at the other end to a label. As indicated, the label linker arms may use various coupling groups to attach the linker arm to the base particle. The label linker arms also from a cleavable label bond with the label which is cleaved to separate the label from the base particle. In one aspect, the label bond is cleavable under label cleavage conditions that do not cleave the target analytes from the analyte complexes. However, if the target analytes are first removed from the analyte complexes and separated from the analyte detection particles, then it may not be required that the label cleavage conditions would not cleave the target analytes. Instead, the target analytes are removed under cleavage conditions which do not cleave the labels from the base particles, which labels may subsequently be cleaved if required.

### E. Affinity Linker Arms

The affinity linker arms are coupled at one end to the base particle and at the other end to an affinity agent. As indicated, the affinity linker arms may use various coupling groups to attach the linker molecule to the base particle.

The affinity linker arms also form a cleavable affinity bond with the target analyte which is cleaved to separate the affinity agent from the base particle. In one aspect, the affinity bond is cleavable under affinity cleavage conditions which do not cleave the labels from the analyte complexes. However, if the labels are first removed from the analyte complexes and separated from the analyte detection particles, then it may be irrelevant whether the affinity cleavage conditions would cleave the target analytes.

### VI. Analyte Detection Particles

The principles described herein are directed to materials, methods and systems for using analytical labels to detect an analyte in a sample suspected of containing analytes of interest, referred to herein as target analytes. The term “detect” is used to broadly cover the coupling of an analyte detection particle with a target analyte. Detection as used herein also refers to any related actions to be taken relating to the target analytes, including, for example, identifying the presence of the target analytes, collecting the target analytes for further evaluation (e.g., visual observation), separating the target analytes from other sample components, and/or measuring the amount of the target analytes (e.g., concentration).

In one aspect, there are provided analyte detection particles comprising base particles to which at least one analytical label and at least one affinity agent for the target analyte(s) have been coupled. The analytical label may be of any type that is useful in detecting the target analytes. The affinity agents are specific for the target analyte(s). The phrase “specific for” refers to the fact that the affinity agents selectively bind the target analyte(s), but do not bind any other components in the sample.

The analyte detection particles couple with the target analytes to form analyte complexes. The analyte complexes are then manipulated to separate the target analytes from the sample and to optionally provide the analytical labels and/or the target analytes for analysis. The analyte detection particles are thereby useful in the identification, collection and analysis of the target analytes. Also provided herein are methods for preparing the analyte detection particles, as well as methods and systems for using the analyte detection particles to detect the target analytes.

The analyte detection particles are prepared by securing label linker arms to a base particle, and labels to the label linker arms. Also, the affinity linker arms are secured to the base particle. In general, the base particles, labels and linker arms are selected based on the target analyte(s). The following provides an example of the preparation of analyte detection particles in which the base particle was an amine nanoparticle and the label was a mass spec label comprising a peptide. The resulting analyte detection particles were useful in the detection of antigens. While this example provides details for production of a particular analyte detection particle suited to detect a specific target analyte, this disclosure is not limited thereby. Variations directed to detecting other target analytes are within the scope of this disclosure. For example, the nanoparticles could instead be microparticles suited for collection in an alternate manner. Also, the linker arms could differ in structure while still incorporating cleavable label and/or affinity bonds.

A particular system referenced herein involves HER2Neu antigen and SKBR cells. Receptor tyrosine-protein kinase erbB-2, also known as CD340 (cluster of differentiation 340), proto-oncogene Neu, Erbb2 (rodent), or ERBB2 (human), is a protein that in humans is encoded by the ERBB2 gene. It is also frequently called HER2 (from human epidermal growth factor receptor 2) or HER2/neu. HER2 is a member of the human epidermal growth factor receptor (HER/EGFR/ERBB) family, and is involved in normal cell growth. HER2/neu may be made in larger than normal amounts by some types of cancer cells, including breast, ovarian, bladder, pancreatic, and stomach cancers. Thus, amplification or over-expression of this oncogene has been shown to play an important role in the development and progression of certain aggressive types of breast cancer. This may cause cancer cells to grow more quickly and spread to other parts of the body. In recent years the protein has become an important biomarker and target of therapy for approximately 30% of breast cancer patients.

SkBr3 (also known as SK-BR-3) is a human breast cancer cell line, isolated by the Memorial Sloan-Kettering Cancer Center in 1970, that is used in therapeutic research, especially in the context of HER2 targeting. SKBR3 overexpresses the HER2 (Neu/ErbB-2) gene product. These cells display an epithelial morphology in tissue culture and are capable of forming poorly differentiated tumors in immunocompromised mice. The SKBR3 cells, and products derived from it, are used often as positive controls in assays for HER2. In addition, the cell line is also a useful preclinical model to screen for therapeutic agents targeting HER2 and to delineate mechanisms of resistance to HER2-targeted therapies.

### Example 1: Mass Label and Antibody

The following exemplifies a method for preparing an analyte detection particle comprising a label and an affinity agent coupled with a NP. In particular, the analyte detection particle comprises a mass label and an antibody. Both the mass label and the antibody are coupled with the particle with a cleavable C—O bond.

### Materials

| | |
|---|---|
| Fmoc-PEG | Fmoc-PEG-SVA 5000 (Lausan Bio Inc) |
| m-PEG-SVA | m-PEG-SVA 2000 (Lausan Bio Inc) |
| Fmoc | fluorenylmethyloxycarbonyl protecting group |
| Sulfo-SMCC | Sulfo-SMCC no weigh 2 mg (Thermo Fischer A29268) |
| HCTU | O-(1H-6-Chlorobenzotriazole-1-yl)-1,1,3,3-tetramethyluronium |
| | hexafluorophosphate (HCTU) (cabinet) Oakwood Chemical 024888) |
| DIPEA | N,N-diisopropylethanolamine (Hunig's base, DIPEA) (Acro Organics |
| | 367841000) |
| DMAP | 4-Dimethylaminopyridine (DMAP) (Acro Organics 148270250) |
| EDC | N-(3-Dimethylaminopropyl)-N′-ethylcarbodiimide hydrochloride (EDC |
| | HCl) stored in small weighed vials at −20 C. |
| DMF | Anhydrous DMF (Has amines in it and needs degasing) (99.8 Extra Dry |
| | over molecular Sieves, Acros Organics 34843100) |
| ACN | Acetonitrile |
| Amine NP | 80 nm silica NPs (aminated) Nanocompix Nano-Xact |
| HMPB | HMPB linker (Advanced Chem Tech RT2010); |
| | 4-(4-hydroxymethyl-3-methyoxyphenoxy) butyric acid |
| AA-5 (2^{nd} best) | Mass label Betaine- A-V-I-V-A. MW 570.75 Celtek Biosciences LLC |
| VI-5 | Mass label Betaine-V-G-I-A-I. MW 570.75 Celtek Biosciences LLC |
| IG-5 | Mass label Betaine- I-I-V-A-G. MW 570.75 Celtek Biosciences LLC |
| VV-5 (Best) | Mass label Betaine-V-V-V-G-V MW 570.75 Celtek Biosciences LLC |
| AV-5 | Mass label Betaine- A-A-L-V-V. MW 570.75 Celtek Biosciences LLC |
| VA-5 | Mass label Betaine-V-V-I-A-A MW 570.75 Celtek Biosciences LLC |
| G-L-5 | Mass label Betaine-G-G-L-L-L MW 570.75 Celtek Biosciences LLC |
| SKBR | SKBR cells were grown to at 2 × 10\hat{ }7 cell/mL as previously described |
| | by Baird Anal Chem 2019 Were incubated in 2% formaldhdye at 4° C. |
| | overnight. Wash pellet 2 X PBS 0.5 L. Resuspend 0.5 mL PBS and 2 uL |
| | Fetal Bovine Serum. Store at 4° C. |
| Ethanolamine | Weighed out in Freezer at 500 mM in 100 uL (Alfa Aesar, Cat # 2491, |
| | ACS grade 99%, FW 61.08 |
| Sulfo-SMCC | Sulfo-Succinimidyl 4-[N-maleimidomethyl]cyclohexane-1-carboxylate) |
| | (Mol Wgt 436.37) (Thermofisher, cat# PG82021) |
| Anti Her 2 neu | anti Her2nue monoclonal antibody (mAb clone NB3, ATCC cat# HB-10205) |
| mAb | 2.9 mg/mL (no BSA) |
| Anti Her 2 neu | anti Her2nue monoclonal antibody (mAb clone NB3, ATCC cat# HB-10205) |
| mAb Biotin | conjugate to Dylight 550 and Biotin at 2.1 mg/mL in BSA |
| Dylight 550 | |
| MEA | 2-mercaptoethylamine HCL (ThermoFischer, cat# 20408) M.W. 113.61 |
| Streptavidin- | Streptavidin-alkaline phosphatas (Invitrogen S921, 2 mg/mL) |
| ALP | |
| E coli pAb | Rabbit polyclonal antibody to E Coli. MyBioSource MBS534321 at 5 |
| | mg/ml (no BSA) |
| K pneumoiae | Rabbit Polyclonal antibody to K Pneumoiae Invitrogen PA1-7226 at 5 |
| pAb | mg/ml (no BSA) |
| S Aureus pAb | Rabbit Polyclonal antibody to S Aureus Invitrogen PA1-7246 at 5 |
| | mg/ml (no BSA) |
| Pseudomonas | Rabbit Polyclonal antibody to Pseudomonas aeruginosa. Abcam |
| aeruginosa pAb | ab68538 at 4.5 mg/ml (no BSA) |

Equipment: Analytical balance & pH meter. Centrifuge: able to work with 50 mL and 1.5 mL tubes. Cup-horn sonicator (500W Qsonica) able to pulse adjust sonication and adjust amplitude with chill bath suspension of NPs in all cases.

### Step 1 Pre-PEGylation and HMPB-NP Coupling in Acetonitrile (ACN)

80 nm aminated silica nanoparticles (SiNPs) were reacted with SVA-PEG5k-Fmoc (0.1 mole eq each) to PEGylate approximately 10% of available amine sites, assuming full reaction extent, leaving ˜10 k free NH2 sites per NP.

### Step 1.1—PEGylation

1. Removed SVA-PEG5 k-Fmoc from freezer and allowed to equilibrate to room temperature.
2. Suspended 100 mg of 80 nm aminated silica nanoparticles (NPs) in 5 mL of deionized water via sonication (60s @ 3s ON, 3s OFF, 50% amplitude) in a 50 mL conical tube.
3. Added 5 mL of ACN and centrifuged (30 min @ 7 k rcf) to pelletize particles on side of tube.
4. Removed and discarded supernatant. Resuspended in ˜5 mL of ACN
5. Prepared a solution of PEG-SVA reagents as follows:
   a. Dissolved 7.5 mg of SVA-PEG5k-Fmoc in 200 microliters of ACN.
6. Added PEG-SVA reagents dropwise to tube containing NPs suspended in ACN while swirling.
7. Allowed solution to react for 1 hour on a sonicator with intermittent sonication (3s ON, 3s OFF, 20% amplitude.
8. Centrifuged to pelletize (30 min @ 7 k rcf). Removed and discarded supernatant.
9. Washed particles 3 times (5 mL each wash) with ACN via sonication (60s @ 3s ON, 3s OFF, 50% amplitude) centrifuged to pelletize (15 min @ 7 k rcf).
10. Finally, resuspended in 5 mL of acetonitrile for a final concentration of 20 mg/mL.

### Step 1.1

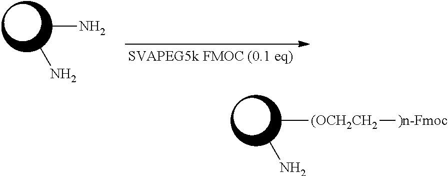

### Step 1.2 HMPB-NP Coupling in Acetonitrile (ACN)

1. Prepared 3 solutions as follows:
   a. 100 mM HCTU in ACN (20.7 mg in 500 microliters ACN)
   b. 100 mM HMPB in ACN (12.0 mg in 500 microliters ACN) and sonicated to dissolve
   c. Neat DIPEA (used glass pasteur pipette to transfer ˜50 microliters into 1.5 mL vial).
2. Mixed 425 microliters of HCTU, 425 microliters of HMPB, and 7 μg of DIPEA in a 1.5 mL eppendorf tube. Allowed to react for ˜5 min.
3. Added solution dropwise to NP suspension while swirling NP suspension.
4. Allowed NP suspension to react for 30 min @ room temperature with intermittent sonication (3s ON, 15s OFF, 20% amplitude).
5. Centrifuged to pelletize NPs (120 min @ 7 k rcf).
6. Removed supernatant and repeated wash 1 additional time with 5 mL ACN and centrifuged to pelletize NPs (30 min @ 7 k rcf).
7. Suspended particles in 10 mL ACN via sonication (60s @ 3s ON, 3s OFF, 50% amplitude) and divided into 10×1 mL aliquots of 10 mg/mL HMPB-NP.

### Step 1.2

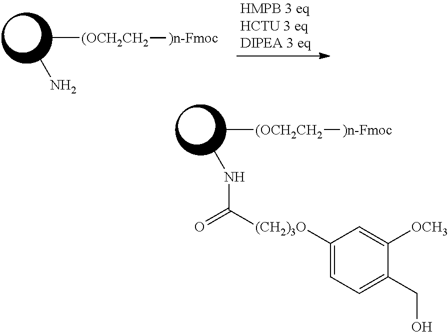

### Step 2 Mass Label Coupling and Deprotection

NPs were then reacted with HMPB linker under HCTU/DIPEA conditions to introduce HMPB linker for cleavable C—O.

### Step 2.1—Performed EDC Activation

1. Weighed ˜0.7 mg of EDC into 1000 μL ACN and vortexed to dissolve.
2. ˜2.2 mg AA-5, IG-5, or VV-5 (or other mass label) into 1000 μL DMSO anhydrous added by glass syringe and vortexed mixture to dissolve.
3. Combined EDC/peptide and vortexed to mix (0.7 mg EDC and 2.0 mg peptide for 10 mg NP).

### Step 2.2—Attached Analytical Label to NP

1. Weighed ˜3.0 mg of DMAP into a 1.5 mL tube and added 1000 μL of ACN and dissolved by vortexing.
2. Added 3 microliters of this DMAP solution to 1 mL of 10 mg HMPB-NPs in ACN (sonicate to mix 5×3 sec ON and 3 sec OFF at 50 amp)
   a. NP solvent—ACN
   b. μg DMAP/mg NP ratio is 0.6 for 3 μL DMAP.
3. While swirling HMPB-NP+ DMAP suspension in 5 mL vial, added all EDC/AA-5 solution dropwise
   a. mg EDC/peptide mg ration is 0.35 for 0.7 mg EDC and 2 mg peptide
   b. AA-5 or VV-5 μg/mg NP ratio is 220 μg/mg NP for 2.2 mg peptide.
4. Incubated at 15 C with intermittent sonication (3s ON, 3s OFF, 50% amplitude) for 20 h.
5. Centrifuged reaction mixture (20 min 7 k rcf) to completely pelletize, removed and discarded supernatant as carefully as possible.
6. Washed 2 times by adding 5 mL water and sonicating at 1 min, 3s ON, 3s OFF, 50% amplitude, and centrifuged at a faster setting (15 min, 7 k rcf).

### Step 2.2

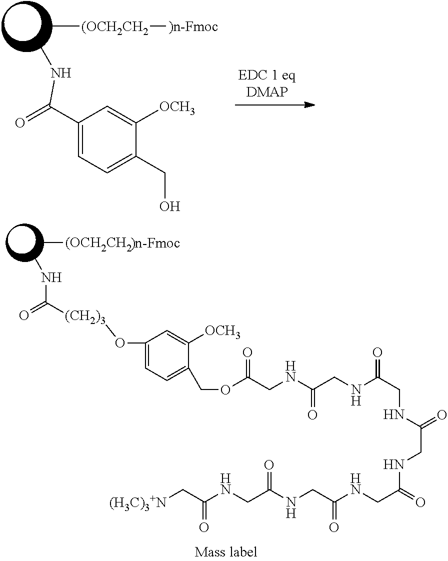

### Step 2.3 Performed Fmoc Deprotection

1. Added 1.0 mL water and 100 μL of 100% ethanolamine and vortexed to mix.
2. Allowed to rest at RT for 20 min with 3 sec ON and 3 sec OFF at 15 C.
3. Centrifuged to pelletize, removed supernatant (15 min, 7 k rcf).
4. Washed particles 5 times with 5 mL water (15 min, @ 7 k rcf).
5. Resuspended 10 mg particles in 1 mL water via sonication (10 mg/mL) (60s @ 3s ON, 3s OFF, 50% amplitude).

### Step 2.3

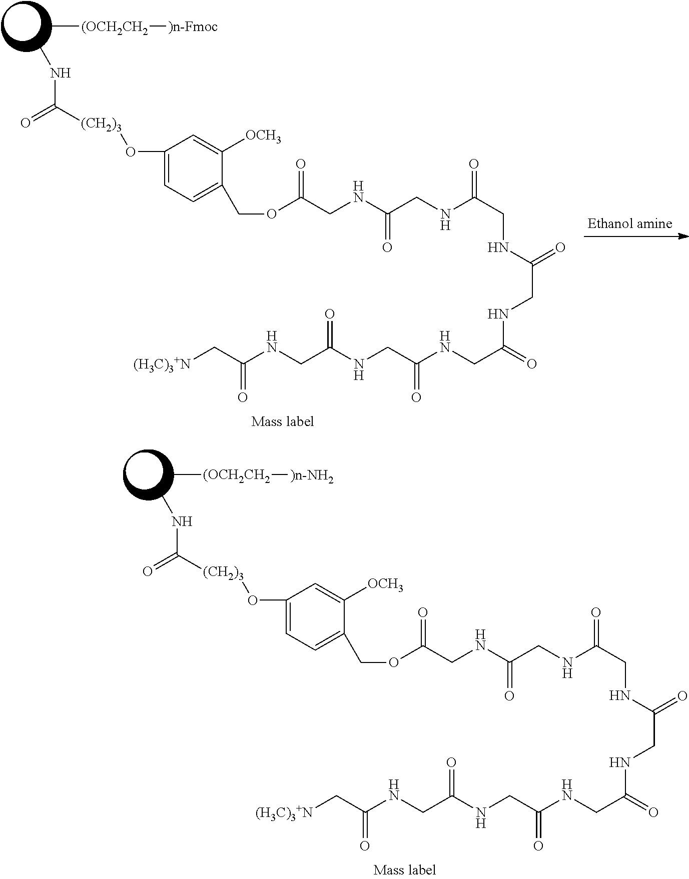

### Part 3—Sulfo-SMCC and Antibody Maleimide Coupling

NPs were then conjugated with analytical labels with a carboxylic acid group as releaseable analytical labels through the cleavable C—O linkage arm.

### Step 3.1 Performed SMCC Addition

1. Removed 200 uL or 2 mg NP particles from the 1 mL sample of 10 mg of NPs in water after sonication (60s @ 3s ON, 3s OFF, 50% amplitude) for mixing.
2. Added to 1.5 mL Eppendorf tubes.
3. Centrifuged to pelletize, removed supernatant (45 min, 12 k rcf) and removed water
4. Added in 90 μL of PBS to the NP.
5. Added 200 μL of water to 2 mg of sulfo-SMCC and then added to 100 μL PBS (Phosphate Buffered Saline) in a 1 mL vial.
6. Added 62 μL of sulfo-SMCC to the 90 μL of PBS and NPs.
7. Sonicated (2.5 hs @ 3s ON, 3s OFF, 50% amplitude) at 4° C.
8. Centrifuged (15 min @ 12 k rcf) and removed PBS, washed 3× with 800 μL of PBS to resuspend particles via sonication (60s @ 3s ON, 3s OFF, 50% amplitude), centrifuged (15 min @ 12 k rcf), and removed PBS.
9. Suspended particles in 50 μL PBS and 10 mM EDTA (40 mg/mL NP final concentration) via sonication (60s @ 3s ON, 3s OFF, 50% amplitude).

### Step 3.1

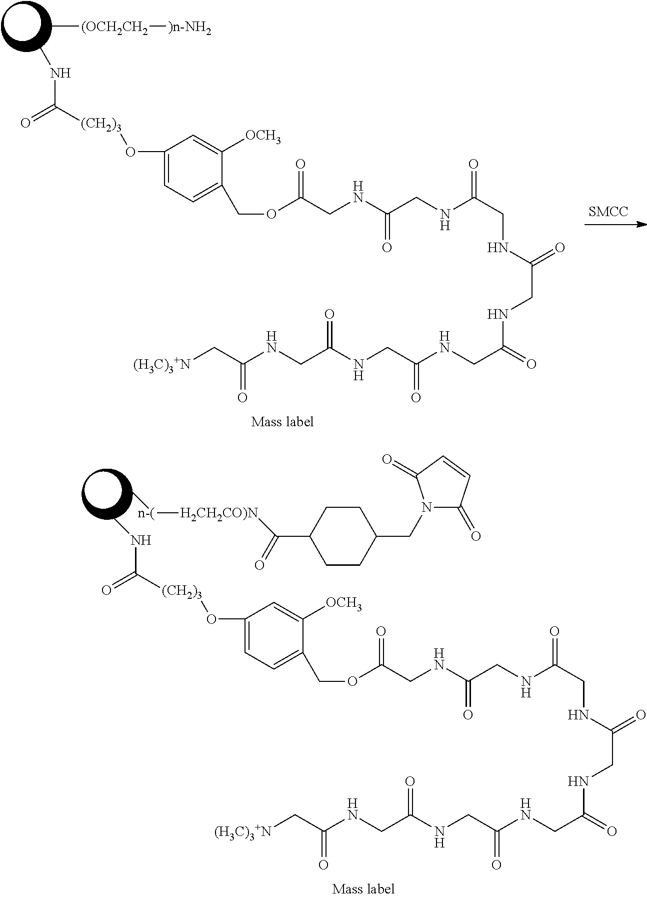

### Step 3.2 Antibody Maleimide Coupling

The nanoparticles were then deprotected and conjugated to a detection antibody via an SMCC/Maleimide coupling. This is a bond that is not cleaved by base or acid or TCEP.
1. Added 100μ of PBS-EDTA to one vial that contains 6 mg of 2-Mercaptoethylamin-HCl (results in 500 mM 2-MEA). Capped and mixed well to dissolve the 2-MEA.
2. Immediately added 3 μL of 2-MEA (500 mM) to 68 μL of Anti Her2Neu mAb (2.9 mg/mL) or ant bacterial pAb (4-5 mg/mL) into tube to make IgG-SH (6.6 mg/mL) by incubation for 90 mins at 37 C.
3. Buffered exchange 2× in PBS-EDTA with Zeba 7 k MWCO, 0.5 mL (1 column total) as follows
   a. The spin columns were prepared by removing columns bottom closure, and loosening but not removing the cap. Placed the column in a 1.5 mL collection tube. Centrifuged at 1500 rcf for 1 min to remove storage buffer
   b. Placed a mark on the side of the column where the compacter resin is slanted upwards.
   c. Placed column in the microcentrifuge with the mark facing outwards in all subsequent centrifugation steps.
   d. Added 300 uL of PBS-ETDA on top of resin and centrifuged at 1500 rcf for 1 min and discarded
   e. Repeated 2 times
   f. Added column to a new tube, removed cap, slowly applied the 68 μL of IgG-SH to center of resin bed
   g. Centrifuged at 1500 rcf for 2 min and recovered the ˜68 μL desalted sample from the bottom of the tube.
4. Added the 136 μL of 6.6 mg/mL IgG-SH (Anti Her2Neu mAb) into tube and added 50 μL of NP (4 mg) in PBS and 10 mM EDTA (40 mg/mL NP final concentration).
5. Incubated reaction mixture by sonicating 3 s ON 15 s OFF, 50% amplitude, 15 C for 30 min.
6. Centrifuged (15 min @ 7 k rcf) and removed conjugation buffer (measured IgG later).
7. Washed in 0.8 mL PBS 3× (7,100×g centrifugation for 12 mins at RT). Sonicated to resuspend.
8. Resuspended NP in 200 μL PBS to a final concentration of 10 mg/mL.
9. Stored as NP-HMPB-5AA-Her2 Neu 10 mg/L).

### Step 3.2

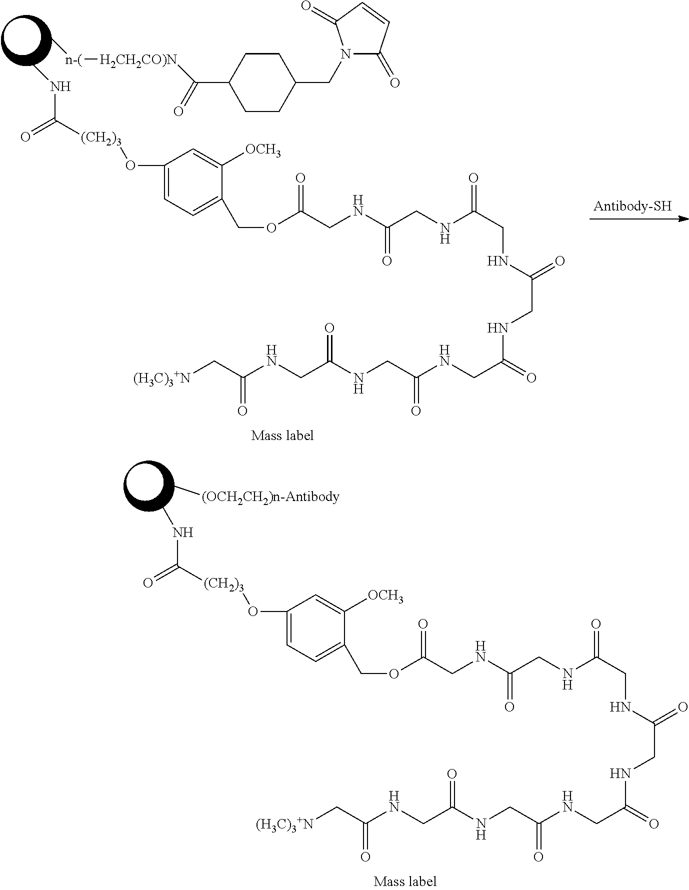

The result is an analyte detection particle comprising a base particle having an appended label attached by a label linker arm, and an appended affinity agent for the target analyte attached by an affinity linker arm. Contacting this analyte detection particle with the target analyte forms an Analyte Complex:

The resulting analyte detection particles include an analytical label coupled with the label linker arm by way of an ether (C—O) bond. This ether bond is cleavable to release the analytical label under label cleavage conditions, as shown below. In one aspect, the label cleavage conditions are based on acid conditions.

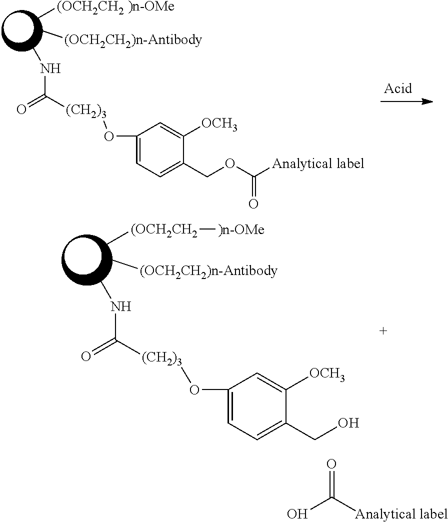

### VII. Analyte Complexes

### A. Formation of Analyte Complexes

The analyte detection particles are incubated with the sample suspected of containing the target analytes. For example, the analyte detection particles of Example 1 were exposed to a sample under conditions to cause coupling of the antibody with the target analyte, e.g., antigen, for which the antibody was specific. The coupling of the target analytes with the analyte detection particles results in formation of analyte complexes.

### B. Detecting Multiple Target Analytes

In some methods, the sample is incubated with analyte detection particles specific to multiple different target analytes. The analyte detection particles are selected in order to couple all of the target analytes of a particular type with the same label. Thus, the target may be a single target analyte or may be the simultaneous detection of each of multiple target analytes. These may constitute different target molecules and/or target cells, and they may or may not include multiple target variants. The analyte detection particles may include an affinity agent specific to each of the intended target analytes, with each different analyte detection particles having a unique affinity agent and label. In this manner, each different target analyte of interest may be individually, yet simultaneously, detected.

The amount of each different affinity agent that is employed is dependent on one or more of the nature and potential amount of each different population of target analyte, the nature of the label, the nature of attachment, the nature of the affinity agent, and/or the nature of the collection particle, if employed. In some aspects, the amount of each different affinity agent may be as low as about 0.001 μg/μL to about 100 μg/μL, or as high as about 0.5 μg/μL to about 10 μg/μL, for example.

Thus, depending on the target analyte(s) of interest, the analyte detection particles may detect a variety of target analyte populations. In this manner, the analyte detection particles can provide detection of a single target analyte, or detection of multiple target analytes, either as a whole or individually.

1. Single Target Analyte

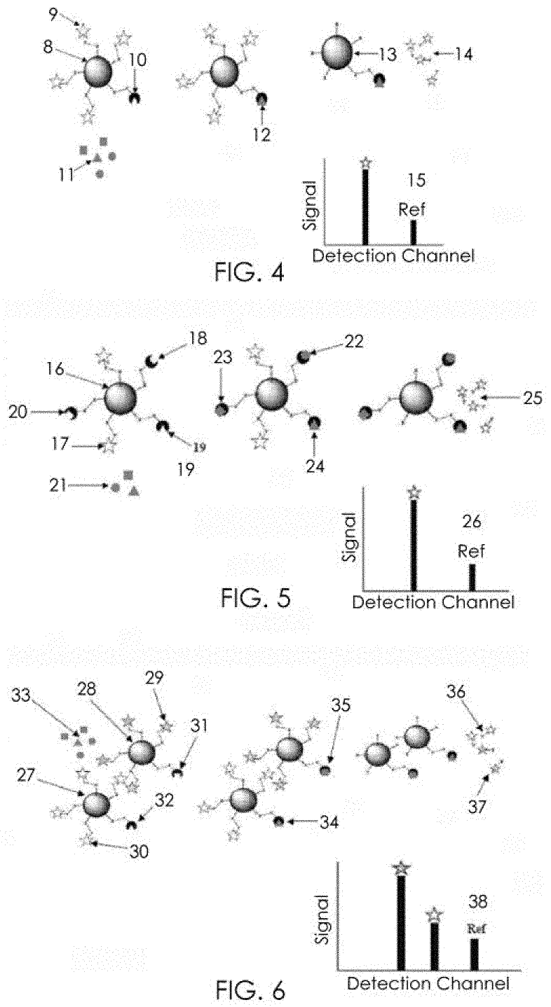

In the most basic aspect, the interest is in a single target analyte. An example of detection of a single target analyte is depicted schematically in FIG. 4. In this example, each analyte detection particle includes the same label and the same affinity agent. The affinity agent is capable of specifically binding to the single target analyte. In the first step, the analyte detection particles with attached affinity agents are incubated with a sample containing the target analyte 11. In a second step, the affinity agent binds to the specific target analyte, as shown at 12, forming analyte complexes. The analyte complexes are then collected and the labels are detected either as bound to the analyte detection particles, or by cleavage of the label linker arms, as shown at 14. As shown, multiple identical labels may be attached to each analyte detection particle in order to provide an amplified response 15.

2. All of Multiple Target Analytes

Referring to FIG. 5, there is shown an example in which multiple different target analytes are detected as a whole. In the first step, the analyte detection particles are incubated with a sample containing the multiple different target analytes. Thus, all of the target analytes are coupled with the analyte detection particles 16 having attached analytical labels 17 and unique, attached affinity agents 18, 19 and 20. For example, the analyte detection particles are incubated with a sample containing different target antigens 21. The analyte detection particles couple with the target antigens as shown at 22, 23 and 24 to form analyte complexes. The analyte complexes are then collected and the labels are detected either as bound to the analyte detection particles, or by cleavage of the label linker arms, as shown at 25. As shown, multiple identical labels may be attached to each analyte detection particle in order to provide an amplified response 26.

In a variation of this example, it is affinity agents that are detected in a sample, such as autoantibodies. In this case the analyte detection particles are incubated with a sample containing the multiple different target antigens. In the first step, the analyte detection particles are incubated with a sample. All of the target analytes are coupled with the analyte detection particles 16 having attached analytical labels 17 and unique, attached target antigens 18, 19 and 20. The analyte detection particles couple with the affinity agents in a sample as shown at 22, 23 and 24 to form analyte complexes. The analyte complexes are then collected and the labels are detected either as bound to the analyte detection particles, or by cleavage of the label linker arms, as shown at 25. As shown, multiple identical labels may be attached to each analyte detection particle in order to provide an amplified response 26.

3. Each of Multiple Target Analytes

A further example for the separate detection of multiple target variants or analytes is depicted in FIG. 6. In the first step, multiple label particles with unique attached affinity agents are mixed with a sample containing differing target analytes. Multiple types of analyte detection particles are used, each type having a unique affinity agent and unique analytical label. In a second step, the affinity agents bind to the target analytes and the analyte detection particles can be captured as is, or bound by collection particles or cells and removed from samples by various means such as size exclusion filtration on a porous matrix, magnetic separation, or centrifugation. In this manner the target variants or analytes bound to particles are separated from particles which are not bound. In a third step, label particles with captured target variants or analytes, such as antigens, are subjected to conditions which release the labels and allow quantifiable detection of multiple released labels within the same sample by comparison to a reference standard.

FIG. 6 is a schematic depicting an example including the isolation of all target analytes in a sample by binding all target analytes to multiple analyte detection particles 27 and 28. The analyte detection particles have attached unique labels 29 and 30 and attached unique affinity agents 31 and 32. When incubated with a solution containing the target analytes, such as antigens 33, the target analytes couple with the affinity agents, as shown at 34 and 35, and are isolated from the sample with intact analytical labels where multiple identical labels are attached to an analyte detection particle.

### Example 2: Multiplexed Mass Labels for Target Analytes

A group of polypeptides with the same 5 amino acids and a molecular weight of 571, were synthetically produced by standard peptide synthesis with a C-terminal carboxylic acid and an N-terminal Betaine. The Betaine served as an ionized charge group for mass spectroscopy. The 5 amino acids were used as releasable mass labels through the cleavable C—O linker arms. Table 1 shows the intensity of mass signal read by the Mass Spectrometer (ThermoFisher LTQ) for the masses shown. Each mass label was able to be read at the same time using a unique mass fragment, (e.g., VV5 was read at a fragment of 472.5). This allowed simultaneous, multiplexed readings. An internal standard peptide, was read at a reference mass of 282.2. All mass labels were connected by a C—O linkage to the nanoparticle and released under acidic condition.

TABLE 1
Multiplexed signal and mass of panel

| | Intensity of | Multiplexed | Ref |
|---|---|---|---|
| Peptide | signal | mass | Mass |
| W5 | 9410.75 | 472.5 | 282.2 |
| AV5 | 840.5 | 352.2 | 282.2 |
| IG5 | 316.3 | 326.3 | 282.2 |
| AA5 | 2873.8 | 296.2 | 282.2 |
| VA5 | 236.85 | 368.4 | 282.2 |

### C. Differing Label Types

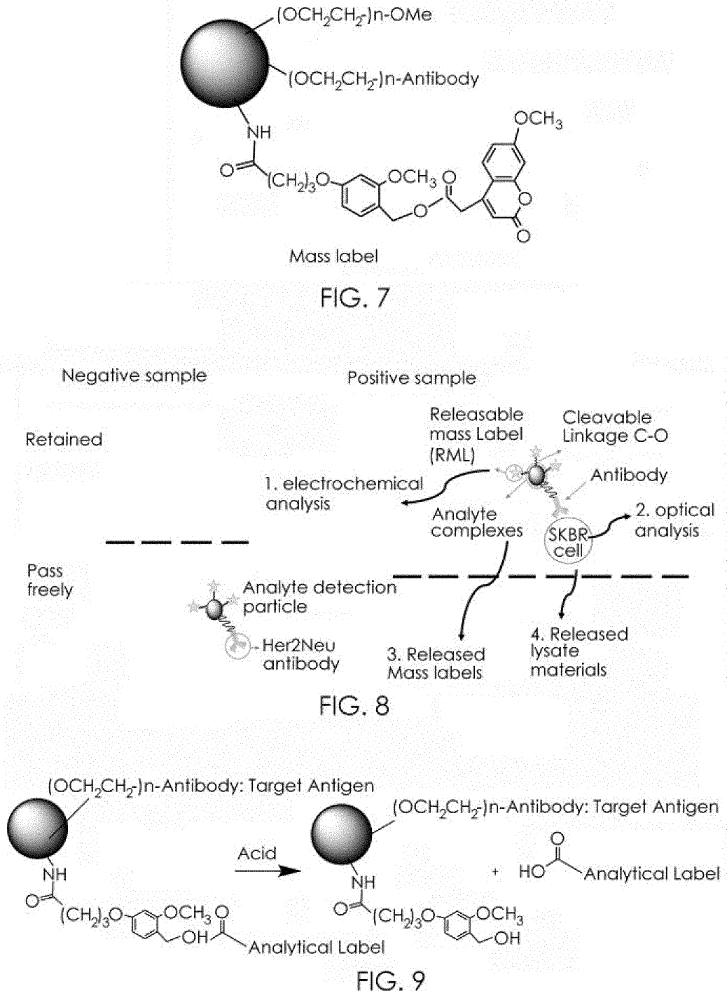

Detection methods using the C—O linkage method were used to demonstrate that the method may be used at the same time with different types of labels, e.g., mass, electrochemical, and optical labels. The optical and electrochemical label, 7-Methoxycoumarin-4-acetic acid (MCAA) was attached through the carboxylic acid group. The label was attached to the nanoparticle as shown in the following FIG. 7. This label is a UV-excitable, blue fluorescent dye with excitation to 351 nm; emission range 430, range ˜410 to 470 nm. Methoxycoumarin-4-acetic acid (MCAA) is also electrochemically active and has been used as an electrochemical label.

The MCAA label was released upon acid treatment while the nanoparticles held at pH 7 did not release the MCAA. The MCAA label was detected at 5 nM upon release from the nanoparticles. The MCAA label was detected electrochemically at 1 nM or as a mass at 0.1 nM. In one example, the labels were read in wells by electrochemical electrodes to determine the location of positive samples, and then optical observation of the cellular structures, and then the mass labels were released and passed through the membrane for marker measurement. In this example, the cancer cells were retained on the membrane along with bound nanoparticles. Bound labels were read by electrodes, and optical labels were read under a microscope upon mass label release.

This example with MCAA using the C—O linkage to attach to the nanoparticles demonstrated that labels can be read simultaneously as electrochemical, mass spectrometric and/or optical read outs. This allows multiple reading types to be taken from the same sample without impacting the C—O— linkage.

### VIII. Collection of Analyte Complexes

The analyte complexes are used to collect only the analyte detection particles associated with the target analytes from the sample. As used herein, the terms “collect” or “collection” refer in part to any manner of separating the analyte complexes from the sample. In one approach, the analyte detection particles may be collected on a matrix through size exclusion, or particles may be separated magnetically or by centrifugation. In another approach, the analyte detection particles may be coupled to capture particles and retained on a substrate prior to being exposed to the target analytes. In another approach, the analyte detection particles may be bound to analyte complexes which are bound to capture particles and may be collected on a matrix through size exclusion, or particles may be separated magnetically or by centrifugation. In another approach, the analytes might be on an immobilized on a solid surface or tissue section and the analyte detection particles reacted and washed by standard slide staining procedure. In another approach the analyte bound may be identified by flow cytometry. In all approaches, the analyte detection particles are collected and may be washed to remove extraneous materials. The labels may be detected in this form, or they may for example be cleaved from the ADP Complexes and collected in a liquid. The target analytes may also be analyzed while coupled in the analyte complexes, or cleaved from the analyte detection particles and then analyzed.

### A. Size Exclusion

In one aspect, the analyte complexes are collected based on size exclusion. A “retention matrix” is used such that the bound target analytes are selectively retained by the matrix. Porous matrices are used where the analyte detection particles are sufficiently smaller than the pore size of the matrix such that physically the particles can pass through the pores. In other examples, the particles are sufficiently larger than the pore size of the matrix such that physically the particles cannot pass through the pores.

In particular, the desired target analytes are separated from other components of the sample based on the sizes of the analyte complexes. Thus, the analyte complexes are such that they are retained on the matrix, while neither the analyte detection particle alone, or the target analyte alone, is retained on the same matrix. Thus, the base particles and/or other components of the analyte detection particles are retained on a matrix once coupled with a target analyte. All of the analyte detection particles selectively bind to the target analytes and are thereby retained on the matrix.

Size exclusion utilizes a “retention matrix” or “matrix” which operates by limiting passage therethrough based on size, referred to herein as retention size. That is, a target analyte of interest has a retention size if it is retained by, rather than passing through, the retention matrix. By way of example, a retention substrate may comprise a porous matrix. The porous matrix may be a solid or semi-solid material, which is impermeable to liquid except through one or more pores of the matrix. The porous matrix is associated with a porous matrix holder and a liquid holding well. The association between porous matrix and holder can be achieved with the use of an adhesive. The association between the porous matrix in the holder and a liquid holding well can be through direct contact or with a flexible gasket surface.

The retention size of the particle is dependent on one or more of the nature of the target molecule, the nature of the sample, the permeability of the cell, the size of the cell, the size of the nucleic acid, the size of the affinity agent, the magnetic forces applied for separation, the nature and the pore size of a filtration matrix, the adhesion of the particle to matrix, the surface of the particle, the surface of the matrix, the liquid ionic strength, liquid surface tension and components in the liquid, the number, size, shape and molecular structure of associated label particles, for example. In some examples the average diameter of the collection particles is at least 1 μm but not more than about 20 μm.

The porous matrix may be a solid or semi-solid material, and may be comprised of an organic or inorganic, water insoluble material. The porous matrix and holder are non-bibulous, which means that it is incapable of absorbing liquid. In some examples, the amount of liquid absorbed by the porous matrix is less than about 2% (by volume), or less than about 1%, or less than about 0.1%, or less than about 0.01%, or 0%. The porous matrix is non-fibrous, which means that the membrane is at least 95% free of fibers, or at least 99% free of fibers, or 100% free of fibers. The matrix does not include fibrous materials such as cellulose (including paper), nitrocellulose, cellulose acetate, rayon, diacetate, lignins, mineral fibers, fibrous proteins, collagens, synthetic fibers (such as nylons, dacron, olefin, acrylic, polyester fibers, for example) or, other fibrous materials (glass fiber, metallic fibers), which are bibulous and/or permeable.

The matrix can have any of a number of shapes such as, for example, a planar or a flat surface (e.g., strip, disk, film, and plate). In some examples the shape of the porous matrix is circular, oval, rectangular, square, track-etched, planar or flat surface, for example. The matrix may be fabricated from a wide variety of materials, which may be naturally occurring or synthetic, polymeric or non-polymeric. The shape of the porous matrix is dependent on one or more of the nature or shape of the holder for the membrane, of the microfluidic surface, of the liquid holding well for example.

The matrix and holder may, for example, be fabricated from plastics such as, for example, polycarbonate, poly (vinyl chloride), polyacrylamide, polyacrylate, polyethylene, polypropylene, poly-(4-methylbutene), polystyrene, polymethacrylate, poly- (ethylene terephthalate), nylon, poly(vinyl butyrate), poly(chlorotrifluoroethylene), poly(vinyl-butyrate), polyimide, polyurethane, and paraylene; silanes; silicon; silicon nitride; graphite; ceramic material (such, e.g., as alumina, zirconia, PZT, silicon carbide, aluminum nitride); metallic material (such as, e.g., gold, tantalum, tungsten, platinum, and aluminum); glass (such as, e.g., borosilicate, soda lime glass, and Pyrex®); and bioresorbable polymers (such as, e.g., polylactic acid, polycaprolactone and polyglycolic acid); for example, either used by themselves or in conjunction with one another and/or with other materials.

The porous matrix for each liquid holding well comprises at least one pore and no more than about 2,000,000 pores per square centimeter (cm²). In some examples the number of pores of the porous matrix per cm² is 1 to about 2,000,000, or 1 to about 200,000, or 1 to about 5,000, or 1 to about 1,000, or 1 to about 100, or 1 to about 50, or 1 to about 10.

The density of pores in the porous matrix is about 1% to about 20%, or about 1% to about 10%, or about 1% to about 5%, or about 5% to about 10%, for example, of the surface area of the porous matrix. In some examples, the size of the pores of a porous matrix is that which is sufficient to preferentially retain liquid while allowing the passage of liquid droplets formed in accordance with the principles described herein.

The size of the pores of the porous matrix is dependent on the nature of the liquid, the size of the cell, the size of the collection particle, the size of analytical label, the size of the target analytes, the size of the label particles, and/or the size of non-target cells, for example. In some examples the average size of the pores of the porous matrices is about 0.1 to about 20 microns, or about 0.1 to about 1 micron, or about 1 to about 20 microns, or about 1 to about 2 microns, for example.

Pores within the matrix may be fabricated in accordance with the principles described herein, for example, by thermal wafer fabrication (Si, Si02), metal oxide semi-conductor (CMOS) fabrication, micro-milling, irradiation, molding, machining, laser ablation and other manufacturing processes for producing microsieves, membranes, macrowells of mm diameters and microwells of um diameters for example, or a combination thereof.

In some cases, the porous matrix is permanently attached to a holder which can be associated with the bottom of a liquid holding well and to the top of a vacuum manifold where the porous matrix is positioned such that liquid can flow from the liquid holding well to the vacuum manifold. In some cases, biological microelectromechanical (BioMEMS) technology is used to apply liquids and vacuums to the porous matrix in the holder. In some examples, the porous matrix in the holder can be associated with a microfluidic surface, top cover surface and/or bottom cover surface. The holder may be constructed of any suitable material that is compatible with the material of the matrix. Examples of such materials include, by way of example and not limitation, any of the materials listed above for the porous matrix. The material for the housing and for the porous matrix may be the same or different. The holder may also be constructed of non-porous glass or plastic film.

Examples of plastic film materials for fabricating the holder include polystyrene, polyalkylene, polyolefins, epoxies, Teflon®, PET, chloro-fluoroethylenes, polyvinylidene fluoride, PE-TFE, PE-CTFE, liquid crystal polymers, Mylar®, polyester, polymethylpentene, polyphenylene sulfide, and PVC plastic films. The plastic film can be metallized such as with aluminum. The plastic films can have relative low moisture transmission rate, e.g. 0.001 mg per m²-day. The porous matrix may be permanently fixed attached to a holder by adhesion using thermal bonding, mechanical fastening or through use of permanently adhesives such as drying adhesive like polyvinyl acetate, pressure-sensitive adhesives like acrylate-based polymers, contact adhesives like natural rubber and polychloroprene, hot melt adhesives like ethylene-vinyl acetates, and reactive adhesives like polyester, polyol, acrylic, epoxies, polyimides, silicones rubber-based and modified acrylate and polyurethane compositions, natural adhesive like dextrin, casein, lignin. The plastic film or the adhesive can be electrically conductive materials and the conductive material coatings or materials can be patterned across specific regions of the holder surface.

The porous matrix in the holder is generally part of a filtration module where the porous matrix is part of an assembly for convenient use during filtration. The holder has a surface which facilitates contact with associated surfaces but is not permanently attached to these surfaces and can be removed. A top gasket may be applied to the removable holder between the liquid holding wells. A bottom gasket may be applied to the removable holder between the manifold for vacuum. The gasket is a flexible material that facilitates a liquid or air impermeable seal upon compression. The holder may be constructed of gasket material. Examples of gasket shapes include flat, embossed, patterned, or molded sheets, rings, circles, ovals, with cut out areas to allow sample to flow from porous matrix to vacuum manifold. Examples of gasket materials include paper, rubber, silicone, metal, cork, felt, neoprene, nitrile rubber, fiberglass, polytetrafluoroethylene such as PTFE or Teflon, or a plastic polymer such as polychlorotrifluoroethylene.

1. Time

Contact of the sample with the porous matrix is continued for a period of time sufficient to achieve retention of bound target analytes on a surface as discussed. The period of time used is dependent on one or more of the nature and size of the different populations of target molecules and/or target cells, the nature of the porous matrix, the size of the pores of the porous matrix, the level of vacuum applied to the sample on the porous matrix, the volume to be filtered, and the surface area of the porous matrix, for example. In some examples, the period of contact may be as short as 1 minute or as long as 1 hour.

2. Vacuum

A pressure gradient (e.g., by way of vacuum) may be applied to the sample on the porous matrix to facilitate passage of non-retained species, and other sample contents through the matrix. The pressure gradient applied is dependent on one or more of the nature and size of the different populations of bound species, the nature of the porous matrix, and the size of the pores of the porous matrix, for example. In some examples, the level of vacuum may be as little as 1 millibar and as much as 100 millibar or more. In some examples, the vacuum is an oscillating vacuum, which means that the vacuum is applied intermittently at regular or irregular intervals, which may range, for example, from 1 second to 600 seconds. In this approach, the vacuum may be oscillated from 0 millibar to about 10 millibar, during some or all of the application of vacuum to the sample. The oscillating vacuum may be achieved using an on-off switch, for example, and may be conducted automatically or manually.

### B. Size Exclusion for Analyte Complexes

The analyte complexes are collected in order to separate the target analyte from the sample. In addition to the analyte complexes present in the test material will be non-target analytes, unbound analyte detection particles and other sample components. In an exemplary process for separation, the analyte complexes are directly separated by means of a retention substrate. The retention substrate is selected such that the analyte complexes are retained while the unbound analyte detection particles pass through. That is, the analyte complexes have a retention size such that the retention substrate retains the analyte complexes.

Referring to FIG. 8, there is shown in diagrammatic form the manner in which the analyte complexes are used to collect the target analytes. As shown on the left side, a sample that is “negative” for the target analyte will have no binding to the analyte detection particles, which pass freely through the matrix (dashed line). However, for a positive sample, analyte complexes are formed and do not pass through the matrix. Instead the combination of the analyte detection particles and the target analyte (SKBR cell) and the analyte detection particle presents a retention size that does not pass through the matrix.

As also represented in FIG. 8, the retained analyte complexes are useful for detection of the target analytes in several ways. As shown at “1’, the mass labels may be examined by electrochemical analysis, even while still coupled in the analyte complex. Similarly, as shown at “2”, the SKBR cells may be studied, such as by optical review, while still combined in the analyte complex.

Alternatively, the analyte complexes are rinsed to remove other sample components and the label and/or may be released from the analyte complexes. For example, as shown at “3” the mass labels may be released and collected in a solution for subsequent analysis by mass spectrometry. In addition, the target analytes (SKBR cells) may be lysed and the released lysate materials pass through the matrix and may be analyzed, as shown at “4”.

FIG. 8 depicts a cell assay for EC-IA, MS-IA and gene assay. The materials are not retained on the 8.0 μm membrane for a sample lacking cells bound by antibodies (left). The materials are retained on the 8.0 μm membrane for a sample having SKBR cancer cells bound by antibodies (right). The electrochemical labels, optical labels and mass labels can be released sequentially, followed by cell lysates being removed for analysis.

The label can be cleaved from the particle, and the associated target analyte, without adversely affecting either one. The target analyte can therefore be separately analyzed. It will be noted that the coupling of the label and of the target analyte with the base particle may be selected to allow the label and/or the target analyte to be cleaved under conditions which do not cleave the other. Also, the label may be cleaved before or after cleaving of the target analyte.

Mass label peptides may be modified such that free amine groups (such as the N-terminal amine) or free carboxyl groups (such as the C-terminal carboxyl group) is altered to be a different functional group. By means of example and not limitation, free amines may be modified to be an acetyl group, formyl group, 9-fluorenylmethyloxycarbonyl (Fmoc), succinyl (Suc), chloroacetyl (Cl—Ac), maleimide (Mal), benzyloxycarbonyl (CBZ), bromoacetyl (Br—Ac), nitrilotriacetyl, terbutoxycarbonyl (Boc), 4-Hydroxyphenylpropionic acid (HPP), Lipoic acid (LA), pegylation, allyloxycarbonyl (Alloc), etc. Example of free carboxyl group modification include but is not limited to amidation (NH2), peptide aldehydes, alcohol peptide, chloromethylketone (CMK), 7-amino-4-methylcoumarin (AMC), p-nitroaniline (pNA), para-nitrophenol (—ONP), hydroxysucinimide ester (—OSu), etc. By way of example and not limitation, modifications to the free amines and/or carboxyl groups may be made for the purpose of increasing ionization efficiency, altering mass spectrometric patterns, generation of isobaric mass label peptides, to introduce functional groups that may be used to couple mass label peptides to label particles, or to alter the mass of the mass label peptide. mass spectroscopy analysis determines the mass-to-charge ratio (m/z) of molecules for accurate identification and measurement. Generation of ions (ionization) may be accomplished by several techniques that include, but are not limited to, matrix-assisted laser desorption ionization (MALDI), atmospheric pressure chemical ionization (APCI), electrospray ionization (ESI), inductive electrospray ionization (iESI), chemical ionization (CI), electron impact ionization (EI), fast atom bombardment (FAB), field desorption/field ionization (FC/FI), thermospray ionization (TSP), and nanospray ionization, for example. The masses monitored by the mass spectrometer by several techniques that include, by way of illustration and not limitation, Time-of-Flight (TOF), ion traps, quadrupole mass filters, magnetic sectors, electric sectors, and Fourier transform ion cyclotron resonance (FTICR), for example. The mass spectroscopy method can be repeated in series (mass spectroscopyn), in which parent ions are selected and subjected to fragmentation, following which the fragments generated within the mass spectroscopy analyzer are measured. Fragments can be subjected to additional fragmentation within the mass spectroscopy analyzer for subsequent analysis. sample processing steps are often performed before mass spectroscopy analysis, such as, by way of example and not limitation, liquid chromatography (LC), gas chromatography (GC), ion mobility spectrometer (Imass spectroscopy), and affinity separation.

### IX. Label Detection

### A. Detecting the Label

The labels can be measured by any suitable method, such as optical, electrochemical, or mass spectrographic methods. The presence and/or amount of each different type of analytical label, whether optical labels, electrochemical labels or mass spectrometry labels, can be used in known fashion to determine the presence and/or amount of each different population of target analytes. The manner of detection of the analytes depends on the nature of the label used.

The labels can be detected when retained on a substrate or matrix. The labels may also be detected when released from the analyte detection particle into an analysis liquid. In some examples, the analytical labels are released from label precursors into the analysis liquid without release of the target analytes. In other examples, the labels are released from analytical label precursors into the analysis liquid with the target analytes also released.

Following analysis of the label, the presence and/or amount of each different label is related to the presence and/or amount of each different population of target molecules and/or target cells. Calibrators are employed to establish a relationship between an amount of signal from a label and an amount of target analytes in the sample.

### B. C—O Linkage Sensitivity

The sensitivity of the detection of labels released by means of the cleavable C—O bond were demonstrated by binding nanoparticles to cancer cell samples. Signal generation labels were attached through the C—O cleavable linkage and an affinity agent for recognition of the biomolecules was attached through a non-cleavable maleimide linkage.

The experimental procedure used was to 1) perform cell isolation by capture onto capture particles with affinity agents to bind the target analyte 2) affinity bind the target analyte to the analyte detection particles to form analyte complexes and 3) release the analytical labels for analysis by mass spectrometric.

In the first step, the target cells captured onto microparticles were spun down and unbound supernatant removed by centrifugation. Target cells used were breast cancer cells (SKBR3, HTB-30) and microparticles used were avidin-coated polystyrene polystyrene (150 μm diameter) with biotin-labeled Her2/neu-specific mAb NB3 clone. In the second step, the target cells captured onto microparticles were treated with analyte detection particles to form the analyte complexes. The analyte complexes were filtered using 37-μm pore size exclusion membrane to remove unbound analyte detection particles and cells. Particles were washed once with 50 μL ammonium acetate (10 mM at pH 7.2) and then the membrane reversed to wash particles off the membrane into a 500 uL of water. Image analysis showed there were 80.9 capture particle per μL of sample. Cancer cells were coupled with ˜25% of the microparticles for 0.202 cells/4 of sample or ˜10 cancer cells in 100 uL. Cells per microparticle were determined from images of cells. Positive and negative samples were prepared containing 10 and 0 cells. The analyte complexes were centrifuged, and liquid removed.

In the third step, mass labels were released from analyte detection particles by breaking the C—O bonds by adding and mixing 100 μL of 0.001% Citric acid at pH 5.2. The VI-5 internal standard was added at 500 nM (0.99 μL). The AA-5/VI-5 ratio and VV-5/VI-5 ratio were measured on the mass spectroscopy for the positive and negative control without nanoparticles (Table 3). The mass ratio used for AA-5 was the 296.1 mass over the VI-5 internal standard mass of 282.2. The mass ratio used for VV-5 was the 472.5 mass over the VI-5 internal standard mass of 282.2 mass. The sampling volume was 20 μL. Release of AA-5 or VV-5 mass labels was accomplished by breaking the C—O bonds coupling with the nanoparticles at pH 5.5 or less but not at pH 7.0 or higher. For comparison, analyte detection particles with AC-5 mass label attached to analyte detection particles by S—S were tested and mass labels accomplished by breaking the S—S bond coupling with the nanoparticles with 5 mM TCEP. The mass ratio used for AC-5 was the 308 mass over the AC-5.2 internal standard mass of 333 mass. Results were compared using analyte detection particles with the same number of ˜2000 mass labels per nanoparticle, and the same amount of 1.2×10¹¹nanoparticles per mg sample.

TABLE 2
Sensitivity comparison of C—O to S—S linkage of mass labels

| | Mass | Mass | Mass | nM mass |
|---|---|---|---|---|
| | label | Ratio | Ratio | label |
| Sample | used | Average | SD | detected |
| Micro-particles | AA-5 | 0.063 | 0.002 | 0.063 nM |
| with SKBR | | | | |
| Micro-particles | VV-5 | 0.144 | 0.013 | 0.101 nM |
| with SKBR | | | | |
| Micro-particles | AC-5 | 1.391 | 0.160 | 1.242 nM |
| with SKBR | | | | |
| Micro-particles | Either AA-5 | 0.00000 | 0.00000 | 0.0000 |
| without SKBR | or VV-5 | | | |
| | or AA 5.2 | | | |

The use of the analyte detection particles with the C—O— linkage using either VV-5 or AA-5 mass label was able to ˜0.1 nM of mass labels liberated from analyte complexes whereas analyte detection particles with the S—S linkage using the AC 5 mass label was able to ˜1.24 nM. This is a significant improvement in mass label detection limits over the prior art approach using a disulfide (S—S) bond.

### C. Demonstrating Improved Sensitivity

The sensitivity for analyte detection of labels in accordance with the present disclosure was compared with the prior art approach using a disulfide (S—S) bond for cleavage of the labels. Peptide mass labels attached to the nanoparticles using the cleavable C—O Linker Arm were compared to this prior art system. The C—O bonds were broken by pH 5.2 weak citrate in a few seconds. The S—S bonds were broken by 5 mM TCEP over 1 h. It was possible to detect at a much lower number of nanoparticles, and the labels needed in the sample to be detected were reduced by 1.9 orders of magnitude. This allowed detecting 10 cells in 100 μL when ˜4,000 NPs were bound to each cell, whereas the prior art S—S method required 188 cells in 100 μL. See Table 3.

TABLE 3
Multiplexed signal and mass of panel

| | | Mass label | | | |
|---|---|---|---|---|---|
| | | peptide/ | | Detection Limit | |
| | Release | Internal | nM | Molecules | Nanoparticles |
| Bond | agent | standard | labels | of label | detected |
| S—S | TECP | AC 5/AC 5.2 | 1.24 | 7.1 × 10\hat{ }10 | 3.6 × 10\hat{ }7 |
| C—O | Cit pH | AA-5/VI-5 | 0.066 | 3.8 × 10\hat{ }9 | 1.9 × 10\hat{ }6 |
| | 5.2 | | | | |
| C—O | Cit pH | IG-5/VI-5 | 0.094 | 5.4 × 10\hat{ }9 | 2.7 × 10\hat{ }6 |
| | 5.2 | | | | |
| C—O | Cit pH | VV-5/VI-5 | 0.101 | 2.9 × 10\hat{ }9 | 2.9 × 10\hat{ }6 |
| | 5.2 | | | | |

Another experiment was conducted with 200 μL of nanoparticle with mass labels (10 mg/mL, 2 mg NP) spun down (10 min, 12 k rfc), liquid removed. This was followed by adding 200 μL of citrate pH 5.2 0.001% (or other acid). The VI Internal standard was added and allowed to stand for 5 sec RT, followed by spinning down the NPs (10 min, 12 k rfc) to remove the liquid for mass spectroscopy analysis. Electrochemical (EC) measurements were accomplished by adding 100 μL pAPP (3 mM) in solution of 0.1 M TRIS buffer pH 9 with 1 mg/mL MgCl, and 0.6 M NaCl allowed to react for 10 min. generated by ALP conversion of 4-aminophenyl phosphate (pAPP) to 4-aminophenol (AP) within a 10 min read out. The results shown in Table 4 demonstrate a significant improvement in electrochemical response in the presence of citric 0.0002% to break the C—O bond over the prior art approach using a TCEP to break the disulfide (S—S) bond. There was no impact to EC response at citric 0.0002%, pH 5.2, but significant suppression when using TCEP to break the disulfide (S—S) bond. Additionally, the disulfide (S—S) was sensitive to electrode current and mass labels cleaved from the NP during the EC response measure whereas the ether (C—)) was sensitive to electrode current at pH 7.0. Finally, there was no suppression of mass label response at pH 3.2 to 5.2 but significant suppression when using TCEP to break the disulfide (S—S) bond. Finally, the time to break the C—O was instantaneous at pH 5.5 or less but significantly longer when using TCEP to break the disulfide (S—S) bond. The C—O cleavage was not observed at pH 7.0 or greater.

TABLE 4
Multiplexed signal and mass of panel

| | | | % Changes | % Changes | | |
|---|---|---|---|---|---|---|
| | | Bond | in mass label | in EC | Time to break | |
| pH | Acid in wqt | broken | response Avg | response | bond | |
| 1.5 | TFA 0.1% | C—O | 50% | −38% | <2 | sec |
| 2.4 | TFA 0.01% | C—O | 29% | −22% | <2 | sec |
| 3.27 | Acetic acid 0.1% | C—O | −8% | −17% | <2 | sec |
| 3.8 | Acetic acid 0.01% | C—O | 4% | −13% | <2 | sec |
| 5.2 | citric 0.0002% | C—O | 0% | 0.0% | <2 | sec |
| | 5 mM TCEP | S—S | −120% | −28% | >1 | h |
| | 50 mM TCEP | S—S | −132% | −53% | >30 | min |

### D. Detecting the Target Analyte

In addition to or in the alternative to the label detection, the target analytes, e.g., cells, may be isolated and analyzed. The target analyte are detected either combined or cleaved from the analyte complexes. Cleavage of the target analyte is similar to that for the labels. However, cleavage is caused under affinity cleavage conditions which differ from conditions cleavage occurs as shown in FIG. 9.

### X. Analyte Collection Particles

In another aspect, collection of the target analytes may be facilitated by coupling them with a larger particle, e.g., a “collection particle”. Collection particles typically have a nominal size of 300 um to 1 um and of organic, inorganic or magnetic composition. The term collection particle is used herein to refer to any type of particle which may be attached to a nanoparticle by means of a collection linker arm forming a cleavable bond with the collection particles. The resulting “collection complex” includes the nanoparticles coupled to and forming a cleavable bond with a collection particle, and coupled to and forming a cleavable bond with an affinity agent for the target analyte. The analyte collection particle couples with the target analyte and facilitates its collection.

The analyte collection particles couple with the target analytes to form “collection complexes”. The collection complexes are then manipulated to separate the target analytes from the sample and to optionally provide the target analytes for analysis. The analyte collection particles are thereby useful in the identification, collection and analysis of the target analytes. Also provided herein are methods for preparing the analyte collection particles, as well as methods and systems for using the analyte collection particles to collect the target analytes.

Organic capture particles may be comprised of polymers including, by way of illustration and not limitation, nitrocellulose, cellulose acetate, poly(vinyl chloride), polyacrylamide, polyacrylate, polyethylene, polypropylene, poly(4-methylbutene), polystyrene, poly(methyl methacrylate), poly(hydroxyethyl methacrylate), poly(styrene/divinyl-5 benzene), poly(styrene/acrylate), poly(ethylene terephthalate), dendrimer, melamine resin, nylon, poly(vinyl butyrate), for example, either used by themselves or in conjunction with other materials including latex. The capture particles may also be composed of carbon (e.g., carbon nanotubes), silica, metals (e.g., gold, silver, and iron, including metal oxides thereof,), inorganic compounds and formed into compositions such as suspensions, hydrogels, colloids, dendrimers, dendrons, liposomes and others.

### Example 3: Collection Particle and Antibody

### Summary:

80 nm aminated silica nanoparticles (SiNPs) were reacted with SVA-PEG5k-Fmoc and mPEG2k-SVA (0.4 mole eq each) to PEGylate approximately 80% of available amine sites, assuming full reaction extent, leaving ˜10 k free NH2 sites per NP. NPs were then reacted with HMPB linker under HCTU/DIPEA conditions to introduce HMPB linker for reactive C—O cleavability. NPs were then conjugated with Biotin as releasable affinity label through the cleavable C—O linkage arm. NPs were then deprotected and conjugated to a collection antibody via a SMCC/Maleimide coupling

### Materials:

| | |
|---|---|
| Fmoc-PEG | Fmoc-PEG-SVA 5000 (Lausan Bio Inc) |
| m-PEG-SVA | m-PEG-SVA 2000 (Lausan Bio Inc) |
| HCTU | O-(1H-6-Chlorobenzotriazole-1-yl)-1,1,3,3-tetramethyluronium |
| | hexafluorophosphate (HCTU) (cabinet) Oakwood Chemical 024888) |
| DIPEA | N,N-diisopropylethanolamine (Hunig's base, DIPEA) (Acro Organics |
| | 367841000) |
| DMAP | 4-Dimethylaminopyridine (DMAP) (Acro Organics 148270250) |
| EDC | N-(3-Dimethylaminopropyl)-N′-ethylcarbodiimide hydrochloride (EDC |
| | HCl) stored in small weighed vails at −20 C. |
| DMF | Anhydrous DMF (Has amines in it and needs degasing) (99.8 Extra Dry |
| | over molecular Sieves, Acros Organics 34843100) |
| ACN | Acetonitrile |
| Amine NP | 80 nm silica NPs (aminated) Nanocompix Nano-Xact |
| Strainers | Reversible Strainers (Stemcell Technologies Inc 27215) for 15 mL |
| | Falcon tubes were purchased in 37 um pore size |
| HMPB | HMPB linker (Advanced Chem Tech RT2010) |
| Biotin | Biotin (Mol Wgt 244.31) (ThermoFischer, Cat # 29129) |
| Sulfo-SMCC | Sulfo-Succinimidyl 4-[N-maleimidomethyl]cyclohexane-1-carboxylate) |
| | (Mol Wgt 436.37) (Thermofisher, cat# PG82021) |
| SKBR | SBKR cells were grown to at 2 × 10\hat{ }7 cell/mL as previously described |
| | by Baird Anal Chem 2019 Were incubated in 2% formaldhdye at 4° C. |
| | overnight Wash pellet 2 X PBS 0.5 L, Resuspend 0.5 mL PBS and 2 uL |
| | Fetal bovine Serum Store at 4° C. |
| Ethanolamine | Weighed out in Freezer at 500 mM in 100 uL (Alfa Aesar, Cat # 2491, |
| | ACS grade 99%, FW 61.08 |
| SA or A MP | Streptavidin or Avidin coated polystyrene microparticles, cross |
| | linked, 1% w/v in diameter of 18, 41, 75, 101, 148 and 196 μm |
| | (Spherotech, Chicago IL) |
| Anti Her 2 | anti Her2nue monoclonal antibody (mAb clone NB3, ATCC cat# HB-10205) |
| neu mAb | 2.9 mg/mL (no BSA) |
| MEA | 2-mercaptoethylamine HCL (ThermoFischer, cat# 20408) M.W. 113.61 |

Equipment: Analytical balance & pH meter. Centrifuge: able to work with 50 mL and 1.5 mL tubes. Cup-horn sonicator (500W Qsonica) able to pulse adjust sonication and adjust amplitude with chill bath suspension of NPs in all cases.

### STEP 1 Pre-PEGylation and HMPB-NP Coupling in Acetonitrile (ACN)

### Step 1.1 PEGylation

1. Removed SVA-PEGSk-Fmoc and mPEG2k-SVA from freezer and allowed to equilibrate to room temperature.
2. Suspended 100 mg of 80 nm aminated silica nanoparticles (NPs) in 5 mL of deionized water via sonication (60s @ 3s ON, 3s OFF, 50% amplitude) in a 50 mL conical tube.
3. Added 5 mL of ACN and centrifuged (30 min @ 7 k rcf) to pelletize particles on side of tube.
4. Removed and discarded supernatant. Resuspended in ˜5 mL of ACN.
5. Prepared a solution of PEG-SVA reagents as follows:
   a. Dissolved 28.4 mg of SVA-PEGSk-Fmoc in 200 microliters of ACN
   b. Dissolved 17.0 mg of mPEG2k-SVA in 200 microliters of ACN
   c. Added solution from 5b to tube containing solution 5a.
   d. Rinsed tube 5b with 2×200 microliter aliquots of ACN and added each rinse aliquot to tube 5a.
6. Added PEG-SVA reagents dropwise to tube containing NPs suspended in ACN while swirling.
7. Allowed solution to react for 1 hour on sonicator with intermittent sonication (2s ON, 15s OFF, 50% amplitude.
8. Added 4 mL of acetonitrile to NP reaction suspension and centrifuged to pelletize (30 min @ 7 k rcf). Removed and discarded supernatant.
9. Washed particles 4 times (10 mL each wash) with ACN via sonication (60s @ 3s ON, 3s OFF, 50% amplitude) centrifuged to pelletize (30 min @ 7 k rcf).
10. Finally, resuspended in 10 mL of acetonitrile for a final concentration of 10 mg/mL. Store at 4 C (DO NOT FREEZE).

### Step 1.1

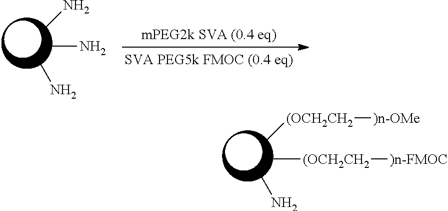

Step 1.2 HMPB-NP coupling in Acetonitrile (ACN)
1. Centrifuged (30 min @ 7 k rcf) to pelletize particles on side of tube.
2. Removed and discarded supernatant. Resuspended in ˜5 mL of ACN.
3. Prepared 3 solutions as follows:
   a. 100 mM HCTU in ACN (20.7 mg in 500 microliters ACN)
   b. 100 mM HMPB in ACN (12.0 mg in 500 microliters ACN)
   c. Neat DIPEA (use glass pasteur pipette to transfer ˜50 microliters of DIPEA into 1.5 mL vial).
4. Mixed 425 microliters of 3a, 425 microliters of 3b, and 5 microliters of 3c in a 1.5 mL Eppendorf tube. Allowed to react for ˜5 min.
5. Added solution from step 4 dropwise to NP suspension while swirling NP suspension.
6. Rinsed sides of 50 mL conical tube with ˜1 mL of ACN to ensure all NPs were in bulk solution volume.
7. Allowed NP suspension to react for 30 min @ room temperature with intermittent sonication (3s ON, 15s OFF, 50% amplitude).
8. Added ACN to bring solution volume to ˜10 mL and centrifuged to pelletize NPs (30 min @ 7 k rcf).
9. Removed supernatant and repeated wash 1 additional time with 10 mL ACN and centrifuged to pelletize NPs (30 min @ 7 k rcf).
10. Finally, suspended particles in 10 mL ACN via sonication (60s @ 3s ON, 3s OFF, 50% amplitude).

### Step 1.2

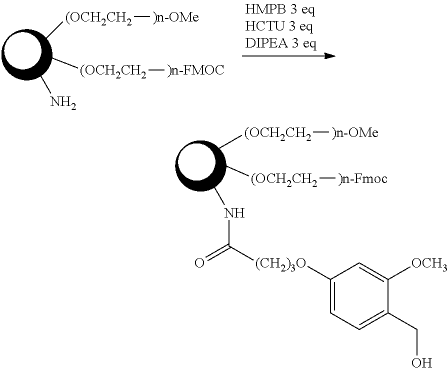

### Step 2 Summary: Biotin Coupling and Deprotection

Step 2.1 Performed EDC activation
1. Weighed ˜14 mg of EDC into a 15 mL tube.
2. Added 3 mL of ACN to EDC and vortexed to mix.
3. Placed ˜25 mg Biotin into a 1.5 mL tube and added 500 uL DMSO and 1.5 mL ACN and vortexed to mix.
4. Added 3 mL of Biotin solution to tube containing 3 mL of EDC.
5. Vortexed to mix.

### Step 2.2 Attached Biotin to NP

1. Weighed ˜4 mg of DMAP into a 1.5 mL tube.
2. Dissolved DMAP in 500 μL of ACN by vortexing.
3. Added 217 microliters of this DMAP solution to 10 mL of 100 mg HMPB-NPs in ACN.
4. While swirling HMPB-NP+ DMAP suspension, added the 6.0 mL EDC/Biotin solution dropwise.
5. Incubated at 15 C with intermittent sonication (3s ON, 15s OFF, 20% amplitude) for 8 h.
6. Centrifuged reaction mixture (15 min, 7 k rcf) to completely pelletize, removed and discarded supernatant as carefully as possible.
7. Washed 1 time with 5.0 mL ACN, centrifuged at a faster setting (15 min, 7 k rcf), finally resuspended in 10.0 mL water for a final concentration of 10 mg/mL.

### Step 2.2

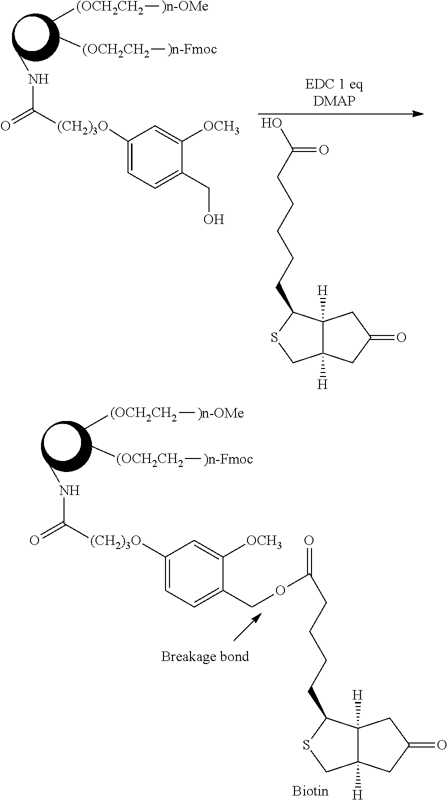

### Step 2.3 Performed Fmoc Deprotection

1. Centrifuged 20 min at 7 rcf and removed ACN and then added 10 mL water and 1 mL of 100% ethanolamine to the 100 mg NP.
2. Allowed to rest at RT for 30 min with sonication to mix at 3 sec on and 15 sec off and 20%.
3. Centrifuged to pelletize, removed supernatant (20 min, 7 k rcf).
4. Washed particles once with ACN (5 mL) with sonication at 1 min, 3 sec on 3 sec off and 20 min @ 7 k rcf) and once with water at same conditions.
5. Resuspended in 10 mL of ACN at a 10 mg/mL concentration NP-HMPB-Biotin-Amine and divided into 10 parts.
6. Labeled as: “NP-HMPB-Biotin; Lot: XXXXYYZZ-MP; 10 mg/mL in Acetonitrile” X=Year, Y=Month, Z=day.

### Step 2.3

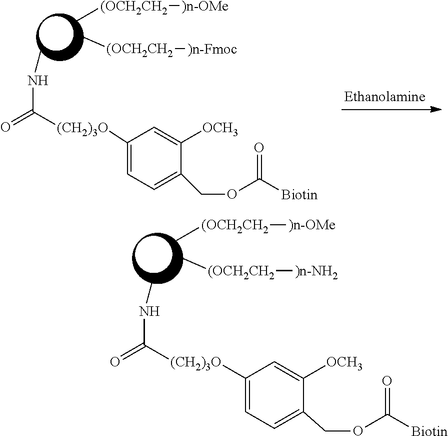

### Part 3 Sulfo-SMCC and Antibody Maleimide Coupling

### Step 3.1 Performed SMCC Addition

1. Removed 100 μL or 1 mg NP particles from the 1 mL sample of 10 mg of NPs in ACN after sonication (60s @ 3s ON, 3s OFF, 50% amplitude) for mixing.
2. Added to 1.5 mL Eppendorf tubes.
3. Centrifuged to pelletize, removed supernatant (15 min, 12 k rcf) and removed ACN.
4. Added in 90 μL of PBS to the NP.
5. Added 200 μL of water to 2 mg of sulfo-SMCC and then added to 100 μL PBS in a 1 mL vial.
6. Added 62 μL of sulfo-SMCC to the 90 μL of PBS and NPs.
7. Sonicated (30 min @ 3s ON, 3s OFF, 50% amplitude) at 15° C.
8. Centrifuged (15 min @ 12 k rcf) and removed PBS, washed 3× with 800 μL of PBS to resuspend particles via sonication (60s @ 3s ON, 3s OFF, 50% amplitude), centrifuged (15 min @ 12 k rcf) and removed PBS.
9. Suspended particles in 50 μl, PBS and 10 mM EDTA (40 mg/mL NP final concentration) via sonication (60s @ 3s ON, 3s OFF, 50% amplitude).

### Step 3.1

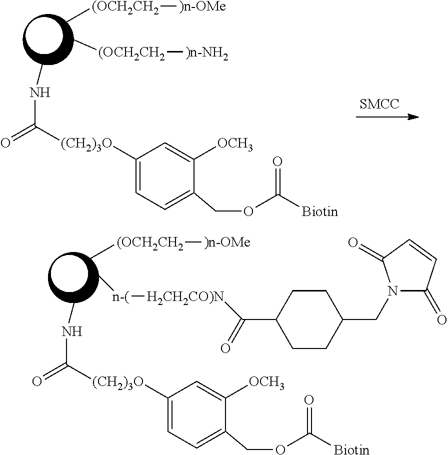

Step 3.2 Antibody Maleimide coupling
1. Added 100 μL of PBS-EDTA to one vial that contains 6 mg of 2-Mercaptoethylamin-HCl (results in 500 mM 2-MEA). Capped and mixed well to dissolve the 2-MEA.
2. Immediately added 3 μL of 2-MEA (500 mM) to 68 μL of Anti Her2Neu mAb into tube to make IgG-SH (6.6 mg/mL) by incubation for 90 mins at 37 C.
3. Buffered exchange 2× in PBS-EDTA with Zeba 7 k MWCO, 0.5 mL (2 columns total) as follows:
   a. The spin columns were prepared by removing columns bottom closure, and loosening but not removing the cap. Placed the column in a 1.5 mL collection tube. Centrifuged at 1500 rcf for 1 min to remove storage buffer
   b. Placed a mark on the side of the column where the compacter resin was slanted upwards.
   c. Placed column in the microcentrifuge with the mark facing outwards in all subsequent centrifugation steps.
   d. Added 300 μL of PBS-ETDA on top of resin and centrifuged at 1500 rcf for 1 min and discarded
   e. Repeated 2 times
   f. Added column to a new tube, removed cap, slowly applied the 71 μL of IgG-SH to center of resin bed
   g. Applied 15 μL of PBS-ETDA on top of resin and centrifuged at 1500 rcf for 2 min and recovered the ˜86 μL desalted sample from the bottom of the tube.
4. Added the 86 μL of 6.6 mg/mL IgG-SH (Anti Her2Neu mAb) into a tube and added 50 μL of NPs (2 mg) in PBS and 10 mM EDTA (40 mg/mL NP final concentration).
5. Incubated reaction mixture by sonicating 3 s ON 15 s OFF, 50% amplitude, 15 C for 30 min.
6. Centrifuged (15 min @ 7 k rcf) and removed conjugation buffer (can measure IgG later).
7. Washed in 0.8 mL PBS twice (7,100×g centrifugation for 12 mins at RT). Sonicated to resuspend.
8. Resuspended NPs in 200 μL PBS to a final concentration of 10 mg/mL.
9. Stored as NP-HMPB-5BIOTIN-Her2 Neu 10 mg/mL).

### Step 3.2

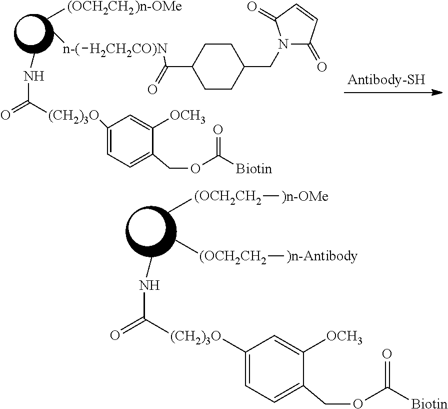

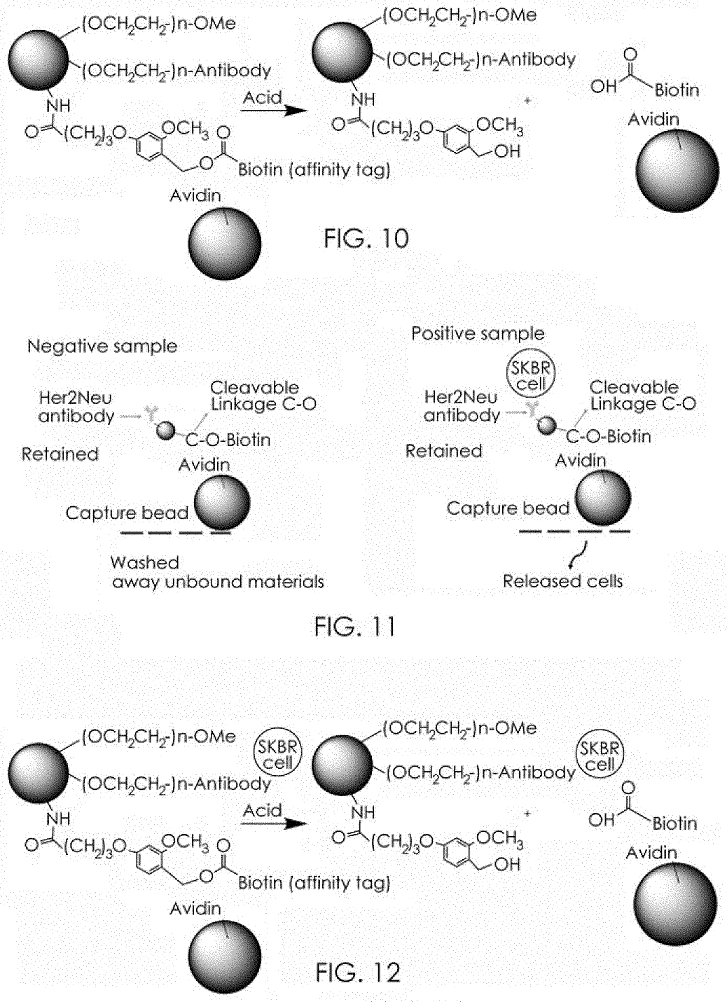

The result is an analyte collection particle comprising a base article having an appended capture particle attached by a capture linker arm, and an appended affinity agent for the target analyte attached by an affinity linker arm. Contacting this analyte collection particle with the target analyte forms a Collection Complex which may be cleaved, as shown in FIG. 10.

### XI. Use of Collection Complexes

The cleavable linker arms are useful in the absence of labeling for the collection and analysis of target cells. As before, analyte collection particles may be used to attach to the target analytes in a manner that allows for separation of the target analytes from the sample. These separation techniques may include those previously described with respect to the analyte detection particles. For example, separation may be effected by attachment of the analyte collection particles to a substrate or by size exclusion techniques using a retention system. Once separated, the target analytes may be cleaved from the analyte collection particles in the same manner as described with respect to the analyte detection particles. The target analytes may then be processed in ways other than by measurement of associated labels. In an alternate embodiment, there are provided materials, methods, and systems for the collection of target analytes in a manner that allows for separation and release of the target analytes in a way that facilitates the use of the target analytes for further analysis.

The Collection Complexes may be prepared in essentially the same way as the Analyte Complexes. The sample is incubated with the analyte collection particles having affinity agents for the target analytes. The analyte collection particles provide a larger effective retention size than the nanoparticles alone. The collection complexes may be collected in various manners, including those already discussed previously.

As shown in FIG. 11, the analyte collection particles couple selectively to the target analytes, e.g., SKBR cells. Through size exclusion, all of the analyte collection particles remain on the retention matrix along with the SKBR cells coupled thereto. Unbound, non-target cells pass through the membrane. As shown, the cells are not retained on the 47.0 μm membrane when not recognized by antibodies attached to the particle (left). The cells are retained on the membrane for a sample with SKBR cancer cells bound by antibodies attached to the particle (right). The SKBR cancer cells are released when the cleavable linkage (C—O) is broken.

The target cells may then be released from the analyte collection particles, as shown in FIG. 12, by cleavage of the collection bond. This collection bond is cleavable under collection cleavage conditions which maintain the target cells viable for analysis. The affinity agent having the cleavable C—O linkage demonstrated capture of SKBR cells. The release of cells from the capture particle was accomplished by breaking the C—O bond coupling the nanoparticle with the biotin. Alternatively, or in addition, the affinity agents for the target cells are attached by affinity linker arms with a cleavable bond which may be cleaved as previously described, thereby separating the target cells from the nanoparticles.

The target analytes may be detected as part of the collection complexes, or separately. In addition or in the alternative, the target analytes may be cleaved from the collection complexes and analyzed. For example, target cells may be isolated and analyzed by visual observation. The target analytes are maintained as viable for analysis under the cleavage conditions.

### Experiment 4: Demonstration of Cell Collection

The affinity collection and release of target cells was demonstrated by binding nanoparticles to streptavidin capture microparticles through Biotin attached to the nanoparticle by a cleavable C—O linkage coupling the collection linker and the collection particle. The test procedure resulted in cancer cells being retained on the surface of the size exclusion membrane until the C—O linkage was broken.

The procedure included 1) cell isolation by affinity binding capture on microparticles, 2) washing unbound material away using size exclusion filtration to capture microparticles but pass unbound cells and nanoparticles, 3) release of cell by cleavage of C—O bond at pH 5.2, and 4) analysis of removed cells by microscope.

The SKBR cells were stained by mixing 100 μL of 2×10\hat{ }5 cell/ml and 5 μL of 1 μg/mL DAPI in 500 μL 0.05% Tween 20 in 10% Candor in PBS (TCPBS) and incubating for 1 min (final is 2×10\hat{ }4). Cells were washed twice with 0.05% Tween 20 in 10% Candor in PBS (TCPBS) using a centrifuge at 2000 rcf and 3 min.

Test samples were prepared by adding 200 μL polystyrene microparticles (PS-NA 150 μm size 0.1% v/w) to 500 μL TCPBS into a 1.5 ml vial for a positive control. To demonstrate the invention, 10 μL AS NP-HMPB-Biotin-Her2 Neu (NP Positive control) or no NP (Negative control) was added for a final concentration of 100 μg/mL, and incubated for 15 min, and incubated for 15 min. Particles were washed twice with 0.05% Tween 20 in 10% Candor in PBS (TCPBS) using a centrifuge at 2000 rcf and 3 min.

The 500 μL of TCPBS were mixed with SKBR cells and another 500 μL was mixed with the polystyrene microparticles containing or lacking nanoparticles. The cell and nanoparticles were put on rocker plate together for 1 h.

Size exclusion filtration was performed by rinsing cells on beads onto reversible Strainers (Stemcell Technologies Inc 27215) for 5 mL Falcon tubes using 37 μm sieve size that pass unbound cancer cell and unbound nanoparticles but retain the polystyrene microparticles containing or lacking nanoparticles and bound SKBR cells. The membrane was blocked and washed with 3×500 μL anti-adherent rinse solution (Stemcell Tech) with waste going into a 5 mL vial, and with a 30 sec centrifugation at 2000 rcf at end to dry the membrane. Add 100 μL of microparticles and cells to top of membrane for each positive and negative control sample. Particles were washed 3×500 μL with 0.05% Tween 20 in 10% Candor in PBS (TCPBS) using a centrifuge at 2000 rcf and 20 sec to dry the membrane. With the microparticles isolated on the membrane, some of the membrane samples were washed with 0.5 mL PBS at pH 7.4 and others were washed with 0.5 mL citric acid (0.001%) at pH 5.2.

The materials remaining on the filters were isolated by reversal of sieve and adding 0.5 ml×3 PBS used to wash captured material into a vial. These collections were spun at 30 sec and 2000 rcf to gather any retained cellular or micro particles into the bottom of the vial. The isolated microparticles were analyzed by imaging using 5 μL of sample placed onto microscope slides. Cell and microparticle counting were performed using a 40× optical zoom and the fluorescence signal from DAPI (Biotek LionHeart Live Cell Imager).

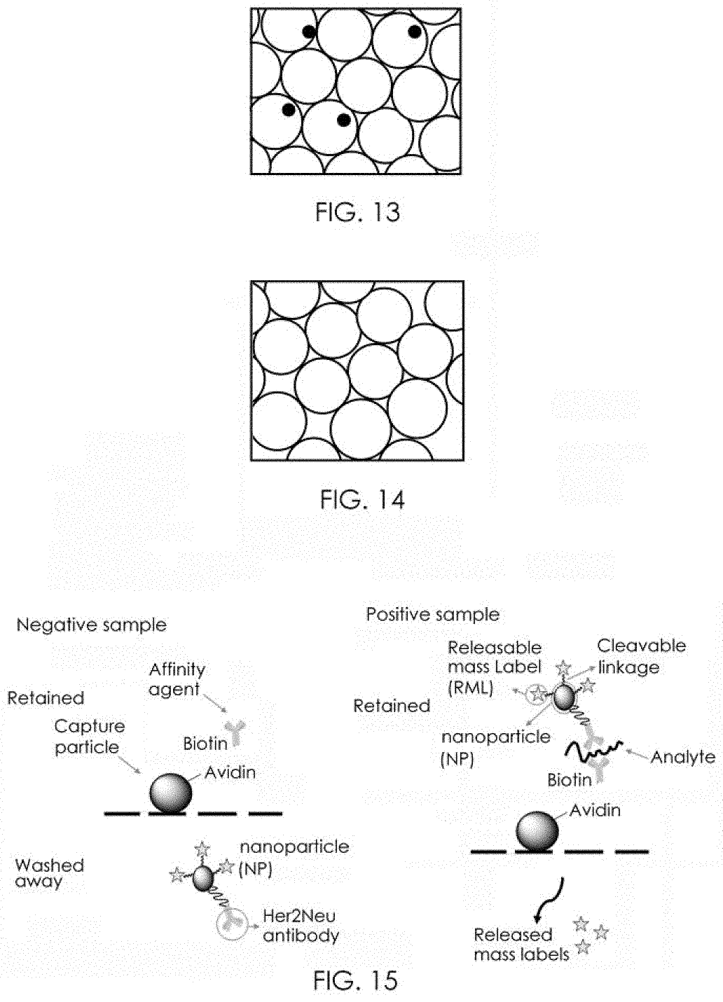

In FIG. 13, the microparticles are shown with the captured SKBR cells when nanoparticles were present and the pH was 7.4. There were 80.9 microparticles per 1 μL of sample and a cancer cell on 1 out of 4 microparticles (0.202 cells/μL of sample). In FIG. 14 the microparticles are shown which did not capture SKBR cells when nanoparticles were not present.

The cells retained on the microparticles captured on the membrane were released by breaking the C—O linkages, releasing all SKBR cancer cells. Cell release by acidic pH was compared to neutral pH (Table 5). Microparticles with and without nanoparticles were compared. The data showed only the microparticles with the nanoparticles captured the cells and only acidic pH released the cells.

| | pH 7.4 treatment | pH 5.2 treatment |
|---|---|---|
| NP with | Cell visible on | Cells visible in |
| C—O-Biotin | microparticles in | microscopy in wash |
| and Her 2-nue | microscopy when | and always without |
| antibody | removed by reversing | microparticle and |
| attached | membrane but not in | nothing removed by |
| | previous wash | reversing membrane |
| No NP with | Cell never on | Cell never on |
| antibody | microparticle sor | microparticles or |
| or biotin | in washes or on | in washes or on |
| attached | membrane | membrane. |

Collection microparticles sized from 10 to 200 μm were further demonstrated with size exclusion membrane pore sizes at 8 to 50 μm. Larger pores of >30 μm with larger capture microparticles >100 μm allowed 20 μm cells to be captured and released. As few as 1 microparticle could be trapped into 1 well with a size exclusion membrane at the bottom.

It was further demonstrated that the size of the well could be adjusted to be just big enough to hold one capture micro particle as long as the diameter of the well was at least 25% larger than the diameter of the microparticles. For example, one 150 μm capture microparticle could be single seeded into a well of 200 μm diameter, 300 μm depth and with a size exclusion membrane on bottom of well. As the microparticles size decreased, the number of microparticles captured increased, as long as the size exclusion membrane allowed retaining the microparticle and the space between the captured microparticle did not present space for passage of biomolecules

The use of Biotin as an affinity label could be extended to any affinity molecule, such as an antibody capable of affinity recognition of a biomolecule. To demonstrate that Biotin could retain a bio-molecule which will then be released, fluorescently labeled streptavidin was bound and then released after exposure to acidic solutions.

The NP-HMPB-Biotin-Her2 Neu in water (200 μL, 2 mg, 10 mg/mL) was spun down and then exposed to 2 μL of streptavidin dylight 488 (1 mg/mL Invitrogen) in 1 mL water for 15 min RT. The nanoparticles were spun down again (15 min 12 rcf) to remove the liquid and leave nanoparticles. The nanoparticles were exposed to 200 μL of different acids or water, spun down 15 min, 12 k rfc) and supernatant removed and released streptavidin measured in in supernatant using a fluorescent plate reader against known concentrations See Table 6.

TABLE 6
Release of Streptavidin Dylight 488 by Breaking C—O Biotin

| | | nM | nM |
|---|---|---|---|
| pH | Acid in wqt | avg | sd |
| 1.5 | TFA 0.1% | 76.49822 | 0.137974 |
| 2.4 | TFA 0.01% | 109.2096 | 5.314609 |
| 3.27 | Acetic acid 0.1% | 51.81356 | 7.315598 |
| 3.8 | Acetic acid 0.01% | 115.5946 | 5.167836 |
| 5.2 | citric 0.0002% | 98.64445 | 6.878375 |
| 5.2 | citric 0.001% | 190.9106 | 3.393223 |
| | blank | 0.066683 | 0.001049 |

Table 6 showed that biotin and the streptavidin were released from the nanoparticles at acidic pH of 5.2 or less. This was true for weak and strong acids. Additionally cells were not damaged above pH 5 and remained intact for further analysis. Previously, it has been shown that S—S linkages to antibodies were unable to cleave and release antibody affinity agents by TCEP treatment once bound to the nanoparticles. In this example, using the C—O linkages allows release from the nanoparticles. As a control the —S—S— linkage was confirmed not to allow release of antibody at 30 min of exposure and 5 mM TCECP.

### XII Combined Detection/Collection Complexes

This test used material made from the prior examples to show how analyte detection particles with labels may be used in combination with analyte collection particles for collection and detection of target analytes. The analyte detection particles can also be bound to capture particles through the analyte complex.

The sample is incubated with the analyte detection particles and the analyte collection particles. The results are complexes including target analytes including both particles. Processing thereafter may include collection of the complexes and detection of the labels and/or analytes generally as previously discussed.

As shown in FIG. 15, the analyte detection particles are not retained on the 8.0 μm membrane for a sample lacking cells bound by antibodies (left). The materials are retained on the 8.0 μm membrane for a sample with analyte bound by antibodies on collection particles and analyte detection particles (right). The mass labels are released and removed for analysis, following removal of unbound materials.

### XIII. Use of Complexes in Sequential Analysis

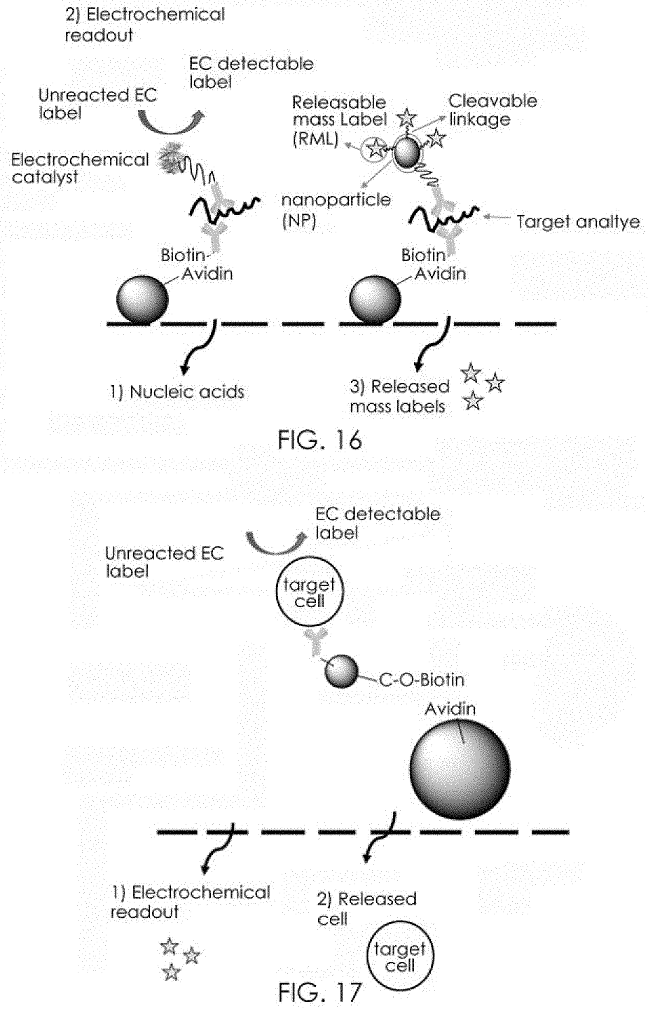

In one example, as shown in FIG. 16, antigens are retained on capture particles with affinity agents using a size exclusion membrane. The analyte detection particles containing releasable analytical labels and affinity agents are retained in detection complexes. Additionally, an electrochemical catalyst (eg. alkaline phosphatase (ALP), horse radish peroxidase (HRP), metals, and other catalyst) capable of generating electrochemical detectable labels is attached to the affinity agent antibodies which are also retained. The unbound materials are removed from the liquid on the top side of the membrane to the bottom side by vacuum, pressure or centrifugal force. The nucleic acid and other materials removed can be analyzed later.

The electrochemical label generates a signal read using positive and reference electrodes placed on the membrane, in microwells or in reacted solution removed. The signal identifies the presence of analyte and captured analyte detection particles and determines whether the next step should occur, which is the addition of acid solution to release the mass labels for multiplexed identification and enumeration. The mass labels are detected after removal from the size exclusion membrane from the top side of the membrane by vacuum, pressure or centrifugal force. Additionally, an electrode placed in the liquid above the membrane can cause spraying of liquid to a reference electrode placed in the mass spectrometer. In other cases, the electrochemical and mass labels are released by acid at the same time. In still other cases, the electro-catalyst is attached to the analyte detection particles. In other cases, positive and reference electrodes placed in the liquid above the membrane can be used to lower the pH of the solution and cause release.

In a second example, depicted in FIG. 17, target cells are retained on capture particles in collection complexes using a size exclusion membrane with affinity agents. The unbound cells are removed. The retained cells are detected by an electrochemical catalyst capable of generating electrochemical detectable labels. The catalyst can be components of the cells (e.g. biomolecules, enzymes) or bound to cells by affinity agents or analyte detection particles. The signal identifies the presence of target cells and determines whether the next step should occur, which is the addition of acid solution to release and remove cells for additional analysis, such as for nucleic acids analysis. In other examples, combined detection/collection complexes are retained and both cell and analytical labels are removed upon acid cleavage.

### XIV Standards

Internal standards are an important aspect of mass spectral analysis. In some examples, a second mass label or structurally similar compound is added to the analysis liquid (as an internal standard) which is used to quantify the mass label used for detection of the target rare molecule. In some instances, the internal standard is isobaric (shares the same parent m/z as the mass label) but exhibits a unique mass spectroscopic pattern when fragmented inside the mass spectrometer. In other cases, the internal standard is selected such that the parent m/z differs slightly from that of the mass label. The internal standards may also contain additional amino acids or derivatized amino acids. Alternatively, the internal standard can be prepared by incorporating one or more isotopic elements such as, but not limited to ²H (D), ¹³C, and ¹⁸O, for example. In such a case, the mass label (or internal standard) has a mass which differs from the naturally-occurring substance. For example, glycerol-C-d7, sodium acetate-C-d7, sodium pyruvate-C-d7, D-glucose-C-d7, deuterated glucose, and dextrose-C-d7, would serve as internal standards for glycerol, sodium acetate, sodium pyruvate, glucose and dextrose, respectively.

In some cases, internal standards and/or isobaric mass labels for multiplexed analyses make use of different peptides with amino acid substitutions such that the nominal molecular weight of the peptide mass labels remain unchanged while fragmentation inside the mass spectrometer results in unique mass spectroscopic signatures for the different mass label peptides. Examples of such peptides include, but is not limited to, amino acid sequences of GAIIR and AAIVR which share a molecular weight of 528.7.7 Da, or RAAVIC and RGIAIC which share a molecular weight of 631.8 Da. In other cases, isobaric mass label peptides and internal standards make use of scrambled amino acid sequences such that fragmentation during mass spectrometric analysis produces one or more unique detectable fragments. Examples of mass label peptides with scrambled amino acid sequences that may be used as internal standards or multiplexable mass labels include, but are not limited to, amino acid sequences of GAIIR, AIIGR, and IGIAR, which all share a molecular weight of 527.7 Da.

### XV Kits

The apparatus and reagents for conducting methods in accordance with the principles described herein may be present in a kit useful for conveniently performing the methods. In one embodiment, a kit comprises in packaged combination affinity agents for one or more different target analytes to be isolated. The kit may also comprise the porous matrix, collection particles, and solutions for spraying, filtering and reacting the analytical labels. The composition of the analyte detection particles may be, for example, as described above. Porous matrices and electrodes may be in an assembly where the assembly can have vents, capillaries, chambers, liquid inlets and outlets. The porous matrix can be removable or permanently fixed to the assembly.

Depending on the method used for analysis of target analytes, reagents discussed in more detail herein below may or may not be used to treat the samples prior to, during, or after the extraction of molecules from the target analytes.

The relative amounts of the various reagents in the kits can be varied widely to provide for concentrations of the reagents that substantially optimize the reactions that need to occur during the present methods and further to optimize the sensitivity of the methods. Under appropriate circumstances one or more of the reagents in the kit may be provided as a dry powder, usually lyophilized, including excipients, which on dissolution provide for a reagent solution having the appropriate concentrations for performing a method in accordance with the principles described herein. The kit may further include a written description of a method utilizing reagents in accordance with the principles described herein.

The spray solvent may be any spray solvent employed in electrospray mass spectroscopy. In some examples, solvents for electrospray ionization include, but are not limited to, polar organic compounds such as, e.g., alcohols (e.g., methanol, ethanol and propanol), acetonitrile, dichloromethane, dichloroethane, tetrahydrofuran, dimethylformamide, dimethylsulphoxide, and nitromethane; non-polar organic compounds such as, e.g., hexane, toluene, cyclohexane; and water, for example, or combinations of two or more thereof. Optionally, the solvents may contain one or more of an acid or a base as a modifier, such as volatile salts and buffer, e.g., ammonium acetate, ammonium bicarbonate, volatile acids such as formic acid, acetic acid, trifluoroacetic acid, heptafluorobutyric acid, sodium dodecyl sulphate, ethylenediamine tetraacetic acid, and non-volatile salts or buffers such as, e.g., chlorides and phosphates of sodium and potassium, for example.

In many examples, the above mentioned spray solvents may be used in combination with aqueous medium, which may be solely water or which may also contain organic solvents such as, for example, polar aprotic solvents, polar protic solvents such as, e.g., dimethylsulfoxide (DMSO), dimethylformamide (DMF), acetonitrile, an organic acid, or an alcohol, and non-polar solvents miscible with water, such as, e.g., dioxane, in an amount of about 0.1% to about 50%. In some examples, the pH for the aqueous medium is a moderate pH ranging from about 4 to about 9. Various buffers may be used to achieve the desired pH and maintain the pH during any incubation period. Illustrative buffers include, but are not limited to, borate, phosphate (e.g., phosphate buffered saline), carbonate, TRIS, barbital, PIPES, HEPES, MES, ACES, MOPS, and BICINE.

Cell lysis reagents are those that involve disruption of the integrity of the cellular membrane with a lytic agent, thereby releasing intracellular contents of the cells. Numerous lytic agents are known in the art. Lytic agents that may be employed may be physical and/or chemical agents. Physical lytic agents include, blending, grinding, and sonication, and combinations or two or more thereof, for example. Chemical lytic agents include, but are not limited to, non-ionic detergents, anionic detergents, amphoteric detergents, low ionic strength aqueous solutions (hypotonic solutions), bacterial agents, and antibodies that cause complement dependent lysis, and combinations of two or more thereof, for example, and combinations or two or more of the above. Non-ionic detergents that may be employed as the lytic agent include both synthetic detergents and natural detergents.

The nature and amount or concentration of lytic agent employed depends on the nature of the cells, the nature of the cellular contents, the nature of the analysis to be carried out, and the nature of the lytic agent, for example. The amount of the lytic agent is at least sufficient to cause lysis of cells to release contents of the cells. In some examples the amount of the lytic agent is (percentages are by weight) about 0.0001% to about 0.5%.

Removal of lipids may be carried out using, by way of illustration and not limitation, detergents, surfactants, solvents, and binding agents, and combinations of two or more of the above. The use of a surfactant or a detergent as a lytic agent as discussed above accomplishes both cell lysis and removal of lipids. The amount of the agent for removing lipids is at least sufficient to remove at least about 50%, or at least about 90%, or at least about 95% of lipids from the cellular membrane. In some examples the amount of the lytic agent is (percentages by weight) about 0.0001% to about 0.5%.

In some examples, it may be desirable to remove or denature proteins from the cells, which may be accomplished using a proteolytic agent such as, but not limited to, proteases, heat, acids, phenols, and guanidinium salts, and combinations of two or more thereof, for example. The amount of the proteolytic agent is at least sufficient to degrade at least about 50%, or at least about 90%, or at least about 95% of proteins in the cells. In some examples the amount of the lytic agent is (percentages by weight) about 0.0001% to about 0.5%.

In some examples, samples are collected from the body of a subject into a suitable container such as, but not limited to, a cup, a bag, a bottle, capillary, or a needle, for example. Blood samples may be collected into Vacutainer® containers, for example. The container may contain a collection medium into which the sample is delivered. The collection medium may be either dry or liquid and may comprise an amount of platelet deactivation agent effective to achieve deactivation of platelets in the blood sample when mixed with the blood sample.

Platelet deactivation agents can be added to the sample such as, but are not limited to, chelating agents such as, for example, chelating agents that comprise a triacetic acid moiety or a salt thereof, a tetraacetic acid moiety or a salt thereof, a pentaacetic acid moiety or a salt thereof, or a hexaacetic acid moiety or a salt thereof. In some examples, the chelating agent is ethylene diamine tetraacetic acid (EDA) and its salts or ethylene glycol tetraacetate (EGTA) and its salts. The effective amount of platelet deactivation agent is dependent on one or more of the nature of the platelet deactivation agent, the nature of the blood sample, level of platelet activation and ionic strength, for example. In some examples, for EDTA as the anti-platelet agent, the amount of dry EDTA in the container is that which will produce a concentration of about 1.0 to about 2.0 mg/mL of blood, or about 1.5 mg/mL of the blood. The amount of the platelet deactivation agent is that which is sufficient to achieve at least about 90%, or at least about 95%, or at least about 99% of platelet deactivation. Moderate temperatures are normally employed, which may range from about 5° C. to about 70° C. or from about 15° C. to about 70° C. or from about 20° C. to about 45° C., for example. The time period for an incubation period is about 0.2 seconds to about 6 hours, or about 2 seconds to about 1 hour, or about 1 to about 5 minutes, for example.

In many examples, the above combination is provided in an aqueous medium, which may be solely water or which may also contain organic solvents such as, for example, polar aprotic or protic solvents such as, e.g., dimethylsulfoxide (DMSO), dimethylformamide (DMF), acetonitrile, an organic acid, or an alcohol, and non-polar solvents miscible with water such as, e.g., dioxane, in an amount of about 0.1% to about 50%, or about 1% to about 50%, or about 5% to about 50%, or about 1% to about 40%, by volume.

An amount of aqueous medium employed is dependent on a number of factors such as, but not limited to, the nature and amount of the sample, the nature and amount of the reagents, the stability of target cells, and the stability of target molecules, for example. In some examples in accordance with the principles described herein, the amount of aqueous medium per 10 mL of sample is about 5 mL to about 100 mL.

Where one or more of the target molecules are part of a cell, the aqueous medium may also comprise a lysing agent for lysing of cells. A lysing agent is a compound or mixture of compounds that disrupt the integrity of the matrices of cells thereby releasing intracellular contents of the cells. Examples of lysing agents include, but are not limited to, non-ionic detergents, anionic detergents, amphoteric detergents, low ionic strength aqueous solutions (hypotonic solutions), bacterial agents, aliphatic aldehydes, and antibodies that cause complement dependent lysis, for example. Various ancillary materials may be present in the dilution medium. All of the materials in the aqueous medium are present in a concentration or amount sufficient to achieve the desired effect or function.

In some examples, it may be desirable to fix the proteins, peptides, nucleic acids or cells of the sample. Fixation immobilizes and preserves the structure of proteins, peptides and nucleic acids and maintains the cells in a condition that closely resembles the cells in an in vivo-like condition and one in which the antigens of interest are able to be recognized by a specific affinity agent. The amount of fixative employed is that which preserves the nucleic acids or cells but does not lead to erroneous results in a subsequent assay. The amount of fixative depends on one or more of the nature of the fixative and the nature of the cells, for example. In some examples, the amount of fixative is about 0.05% to about 0.15% or about 0.05% to about 0.10%, or about 0.10% to about 0.15%, for example, by weight. Agents for carrying out fixation of the cells include, but are not limited to, cross-linking agents such as, for example, an aldehyde reagent (such as, e.g., formaldehyde, glutaraldehyde, and paraformaldehyde,); an alcohol (such as, e.g., C₁-C₅ alcohols such as methanol, ethanol and isopropanol); a ketone (such as a C₃-C₅ketone such as acetone); for example. The designations C₁-C₅ or C₃-C₅ refer to the number of carbon atoms in the alcohol or ketone. One or more washing steps may be carried out on the fixed cells using a buffered aqueous medium.

In examples in which fixation is employed, extraction of nucleic acids can include a procedure for de-fixation prior to amplification. De-fixation may be accomplished employing, by way of illustration and not limitation, heat or chemicals capable of reversing cross-linking bonds, or a combination of both, for example.

In some examples utilizing the techniques, it may be necessary to subject the rare cells to permeabilization. Permeabilization provides access through the cell membrane to nucleic acids of interest. The amount of permeabilization agent employed is that which disrupts the cell membrane and permits access to the nucleic acids. The amount of permeabilization agent depends on one or more of the nature of the permeabilization agent and the nature and amount of the rare cells, for example. In some examples, the amount of permeabilization agent by weight is about 0.1% to about 0.5%. Agents for carrying out permeabilization of the rare cells include, but are not limited to, an alcohol (such as, e.g., C₁-C₅ alcohols such as methanol and ethanol); a ketone (such as a C₃-C₅ketone such as acetone); a detergent (such as, e.g., saponin, Triton® X-100, and Tween®-20); for example. One or more washing steps may be carried out on the permeabilized cells using a buffered aqueous medium.

While the invention has been illustrated and described in detail in the drawings and foregoing description, the same is to be considered as illustrative and not restrictive in character, it being understood that only the representative embodiments have been shown and described and that all changes, equivalents, and modifications that come within the spirit of the inventions defined by the claims are desired to be protected. All publications, patents, and patent applications cited in this specification are herein incorporated by reference as if each individual publication, patent, or patent application were specifically and individually indicated to be incorporated by reference and set forth in its entirety herein.

1. An analyte detection particle for use in performing an analysis of target analytes, comprising:
a particle;
a label;
a label linker arm coupled to the particle and to the label, the label linker arm and the label being joined by a label bond cleavable to separate the label from the particle;
an affinity agent having an affinity for the target analyte and being functional to couple with the target analyte; and
an affinity linker arm coupled to the particle and to the affinity agent, the affinity linker arm and the affinity agent being joined by an affinity bond;
the label bond being cleavable under label cleavage conditions which do not cleave the affinity bond and which leave the cleaved labels and the target analytes attached to the particles through the analyte linker arm viable for analysis.

2. The analyte detection particle of claim 1 in which the label bond is an ether bond that is cleavable under the label cleavage conditions.

3. The analyte detection particle of claim 2 in which the affinity bond is cleavable under affinity cleavage conditions which leave the target analyte viable for analysis, the affinity cleavage conditions being different from the label cleavage conditions.

4. The analyte detection particle of claim 3 in which the affinity bond is an ether bond that is cleavable under the affinity cleavage conditions.

5. The analyte detection particle of claim 4 in which the target analyte is a target cell, the analyte detection particle having a first retention size, the analyte detection particle forming an analyte complex upon coupling of the target cell with the affinity agent, the analyte complex having a second retention size, the second retention size being larger than the first retention size such that the first retention size allows passage of the analyte detection particle through a retention substrate while the second retention size prevents passage of the analyte complex through the same retention substrate.

6. The analyte detection particle of claim 4 and which further includes:
a collection particle; and
a collection particle linker arm attached to the particle and to the collection particle.

7. The analyte detection particle of claim 6 in which the collection particle linker arm is joined to the collection particle by a collection bond cleavable to separate the collection particle from the particle; the collection bond being cleaved under collection cleavage conditions which are different from the affinity cleavage conditions.

8. The analyte detection particle of claim 7 in which the collection bond is an ether bond that is cleaved under the collection cleavage conditions.

9. The analyte detection particle of claim 8 in which the collection cleavage conditions are also different from the label cleavage conditions.

10. The analyte detection particle of claim 9 in which the target analyte has a first retention size and the analyte detection particle has a second retention size, the second retention size being larger than the first retention size such that the first retention size allows passage of the target analyte through a retention substrate while the second retention size prevents passage of the analyte detection particle through the same retention substrate.

11. The analyte detection particle of claim 4 comprising multiple labels attached to the particle by multiple label linker arms.

12. The analyte detection particle of claim 4 comprising multiple different types of labels attached to the particle by multiple label linker arms and multiple different types of affinity agents attached to the particle by multiple affinity linker arms, at least a first type of label type corresponding to a first type of affinity agent and at least a second type of label corresponding to a second type of affinity agent.

13. The analyte detection particle of claim 1 having the Structure III: 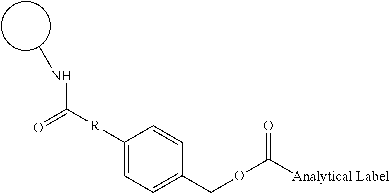
where R is a non-interfering organic group comprising alkyls, polyamides, polypeptides polyethers and other polymeric chains.

14. A method of detecting a target analyte in a sample, comprising:
combining the sample containing the target analyte with a composition containing the analyte detection particles of claim 4 to yield non-complexed analyte detection particles not joined with target analytes and analyte complexes comprising analyte detection particles joined with target analytes;
isolating the labels of the analyte complexes from the non-complexed analyte detection particles; and
measuring the labels.

15. The method of claim 14 in which isolating the labels comprises cleaving the label bonds to separate the labels from the analyte complexes, and collecting the separated labels.

16. The method of claim 15 and which further includes cleaving the affinity bonds to separate the target analytes from the analyte complexes, and collecting the separated target analytes.

17. A method for preparation of analyte detection particles comprising:
reacting a functionalized particle with first and second linker arms having the Structure VI: 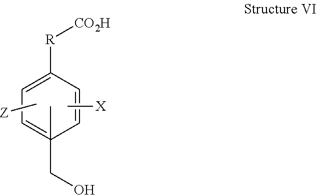
in which R is a non-interfering organic group comprising alkyls, polyamides, polypeptides polyethers and other polymeric chains, and X and Z are optional non-interfering organic groups or hydrogen;
bonding a label to the first linker arm by formation of an ether bond between the label and the linker arm using the oxygen of the CH₂OH group of the first linker arm; and
bonding an affinity agent to the second linker arm by formation of an ether bond between the affinity and the linker arm using the oxygen of the CH₂OH group of the second linker arm.

18. An analyte collection particle for capturing a target analyte, comprising:
a particle;
an affinity agent having an affinity for the target analyte and being functional to couple with the target analyte;
an affinity linker arm coupled to the particle and to the affinity agent, the affinity linker arm and the affinity agent being joined by an affinity bond cleavable under affinity cleavage conditions, the affinity bond being an ether bond;
a collection particle; and
a collection linker arm coupled to the particle and to the collection particle, the collection linker arm and the collection particle being joined by a collection bond cleavable under collection cleavage conditions which are different from the affinity cleavage conditions, the collection bond being an ether bond.

19. A method for capturing target analytes from a sample, comprising:
combining the sample containing the target analyte with a composition containing the analyte detection particles of claim 18 to yield collection complexes comprising analyte collection particles joined with target analytes, and non-complexed analyte collection particles not joined with target analytes; and
isolating the collection complexes from the sample.

20. The method of claim 19 which further includes cleaving the collection bonds and collecting the cleaved target analytes.
